SEQUENCE LISTING (80 sequences)

SEQ ID NO: 1 - DNA, 1341 bp, Glycaspis brimblecombei
aacttggaacctgacaatgttggtgttgtagtattcggtaatgatagattaatcaaggaaggagacattgtcaaacgtactggtgctattgttgatgtacctgttggtgaagatttgttgggaagagtagttgatgctttaggtaacaccattgatggaaaaggaccactcacctctaaaactcgtttccgtgttggaatcaaggcccctggaatcattccccgtatttctgtaagagaacctatgcaatctggtattaaagctgtagattccttggtaccaattggtcgtggtcaacgtgagttgatcattggagatcgtcaaactggaaaaactgctttggccattgataccatcatcaaccaaaagagattcaatgactctgatgacgaaaagaaaaagttgtactgtatctatgttgctattggtcaaaagagatctactgtagctcaaatcgtaaaacgtttaactgactctggtgccatgaaatacaccatcattgtatcagctaccgcctctgatgctgcccctctacaatacttggctccttactctggatgtgccatgggagaattcttccgtgacaatggaaaacacgccttgatcatctttgacgatttatcaaaacaagctgttgcttaccgtcaaatgtctctgctgcttcgtcgtcccccaggtcgtgaagcttaccccggagatgtattttatcttcactctcgtcttcttgaacgttccgctaaaatgtctgaagcccatggaggtggttctttaactgctttacccgttattgaaactcaagctggagatgtatcagcttatatcccaaccaatgtaatttctattactgatggacaaattttcttggaaactgaattgttctacaaaggtattcgtcccgctatcaacgtaggattgtctgtatcccgtgtaggatctgctgcccaaaccagagccatgaaacaggttgccggttcaatgaaattggaattggcccaataccgtgaagtcgccgcttttgcccaattcggttctgatttagatgctgctacccaacaattgctaaaccgtggtgttcgtttgactgaattgttgaaacaaggtcaatatgtaccaatggctattgaagaacaagttgctgtcatctactgtggtgtccgtggtcacttggacaaattagacccagcaaagatcaccacttttgaaaaagaattcttagctcacattaaaacttccgaaaaagctttattggaaagtatcaagaaagaaggaaaaatcactgaagataccgatgctaagttgaagactgttgtacagaacttccttgctaacttcactggttag

SEQ ID NO: 2 - DNA, 100 bp, Glycaspis brimblecombei
aaacgtttaactgactctggtgccatgaaatacaccatcattgtatcagctaccgcctctgatgctgcccctctacaatacttggctccttactctggat

SEQ ID NO: 3 - DNA, 753 bp, Glycaspis brimblecombei
atgagtagctccagatatgaccgggccatcactgtgttctctcctgatggtcatttacttcaagttgaatatgcccaagaagctgtcagaaaaggatcaactgctgttggagtccgtggagacaatgttgtagttctgggtgttgaaaagaaatcagtggcaaaattacaagaagaaagaactgttagaaaaatatgtttacttgatgatcatgttgtcatggcatttgctggtttgacagctgatgctcgtatattaattaatcgtgcacaaattgaatgtcagtctcacaaattgactgttgaagatcctgttacattagaatatattactaggtatattgctggtttgaagcaaaaatatactcaaagtaatggaagaagaccttttggtatatcatgtttgattggaggatttgattatgatggaaaagcaagactatatcaaactgaaccttctggcatttattatgaatggaaggctaatgcaacaggaagaagtgctaagacagttcgtgaattcttagagaaatattataaagctgaagaactaaccacagaaaaggctacagttaaattagcaatacgggccttactagaagtagtacaatctggacaaaagaatctagaaattgctgtcatgaggcatggaaagcctatggagatgttgactgcagctaaaatagaagaatatgttattgaaattgaaaaagaaaaggaagaagaagcagaaaagaaaaagcaaaagaaatag

SEQ ID NO: 4 - DNA, 100 bp, Glycaspis brimblecombei
aatctggacaaaagaatctagaaattgctgtcatgaggcatggaaagcctatggagatgttgactgcagctaaaatagaagaatatgttattgaaattga

SEQ ID NO: 5 - DNA, 789 bp, Glycaspis brimblecombei
atggctaggagatatgattcacgtacaacaatcttctccccagagggacgattgtatcaagtagaatatgctatggaagctatcagtcatgctggtacttgtttgggtatcctagctaatgatggcattctgctggcagccgagaaaagaaacaccaataaattactagatgaaggaaattcatctgagaaaatttacaagttgaatgataatatggtttgcagtgtagctggtattacttcagatgctaatgttctaacatcagaattgagactgatagctcaacgttatttaattcaatatgatgaacccataccttgtgaacaactggtatcttggttatgtgatatcaaacaaggatatactcaatatggaggaaaaagaccgtttggtgtatcaattctgtatatgggttgggataaacagtatggctaccaattatatcaatcagatcctagtggaaactacagtggatggaaagcaacatgtattggaaataatagtgcagctgctatttctaatttgaaacaagagtataaggaagatttgactttagataatgccaagcttttagctatcaaagttctcagtaaaatattggatatgacaaaactaactccggagaaagttgaactggcaacacttacaagaaaagatggcaaaacttttactaaaattttatcagcaaacgaagttgaagctttgatcgctgctcatgagaaagcagaaagtttagaaaaagagaaagaaaaacaagcaaaggctgctgctgctagctcttcttcttag

SEQ ID NO: 6 - DNA, 100 bp, Glycaspis brimblecombei
cgaagttgaagctttgatcgctgctcatgagaaagcagaaagtttagaaaaagagaaagaaaaacaagcaaaggctgctgctgctagctcttcttcttag

SEQ ID NO: 7 - DNA, 366 bp, Glycaspis brimblecombei
cgaacagctggtggcactgtttgggaagatccaacacttcttgaatgggaagatgatgattttcgactgttttgtggagatttaggaaatgacgtcacagatgaattactaattagaaccttttcaaaatatccttcatttttaaaggccaaagttgttcgagataaaagaacaaataaaacaaaaggttttggatttgttagttttaaagatcctcaagattttattcgtgcaaataaagaaatgaatggaagatatgttggtagtcgccctattaaactaagaaaaagtaattggagaaaccgaagtttagaagttgtagaaaaaagagaagaaaaagcaactctgattggtctgctcacaggt

SEQ ID NO: 8 - DNA, 100 bp, Glycaspis brimblecombei
tcgccctattaaactaagaaaaagtaattggagaaaccgaagtttagaagttgtagaaaaaagagaagaaaaagcaactctgattggtctgctcacaggt

SEQ ID NO: 9 - DNA, 561 bp, Glycaspis brimblecombei
gaagcctacttagtgccacgaattgtcctctacagagatgtcatgtatgactctgaaattgatcttatcaagaaaatggctcaacctagacttcgtagagcaacagtacaaaattataaaactggagagttagaaattgcaaattatagaatcagcaaatcagcatggttaagagaaccagaacatccagttgtagaaagaatcagcagaagagttgaagatatgactggacttaccactgaaactgctgaagaacttcaagttgttaactatggaattggtggtcactatgaacctcattatgactttgccaggcctggtgaagccaacgcattcaaatctttaggaactggcaacagagtagcaacagtattattttatatgagtgatgtatctcagggaggggcaacagtttttacttctttaaatttatcattgtggccagaaaaaggaactgcagctttttggcacaatcttcactcaagtggggacggaaattatctaactagacatgctgcttgtccagttcttacaggatcaaaatgggtatcaaacaaatgg

SEQ ID NO: 10 - DNA, 100 bp, Glycaspis brimblecombei
gaagcctacttagtgccacgaattgtcctctacagagatgtcatgtatgactctgaaattgatcttatcaagaaaatggctcaacctagacttcgtagag

SEQ ID NO: 11 - DNA, 529 bp, Glycaspis brimblecombei
aaaggaacaactggtactcaagcttcttttatggaactttttaatggagatggcgaaaaggtgaaagctttagaccgtctggttaccaaaaaagctggtttctctacttctcacatcatctgtggccaaacatactctagaaaagttgacgtcatcgtaacgggagctctcagcagtctaggtgccacaattcacaagcttgcaacagatttacgtttgttagcacatatgaaagaagttgaagagccttttgaatcaactcaaattggttccagtgcaatggcctataaaaggaaccctatgagaagtgagagactgtgttctttagcaagattcctaatgagtttacatcaaaactcattgaacactgccagtacacagtggatggaacgtactcttgatgatagtgctaacaggagacttactctatccgaatcattcctcaccgcagactgccttttaatgacccttcaaaatgttttagaaggattagtagttaataaaaagttattcagcgtcacattgatac

SEQ ID NO: 12 - DNA, 100 bp, Glycaspis brimblecombei
agctttagaccgtctggttaccaaaaaagctggtttctctacttctcacatcatctgtggccaaacatactctagaaaagttgacgtcatcgtaacggga

SEQ ID NO: 13 - DNA, 100 bp, Glycaspis brimblecombei
agctttagaccgtctggttaccaaaaaagctggtttctctacttctcacatcatctgtggccaaacataccctagaaaagttgacgtcatcgtaacggga

SEQ ID NO: 14 - DNA, 894 bp, Glycaspis brimblecombei
aaatcacatgtagaatgtgcccggttttcaccagatggtcaatatttaatcacaggttcagtagatggatttattgaagtttggaattttacaactggaaaaatcagaaaagatttgaaatatcaagctcaggataacttcatgttgatggaagaagccgtcatgtctcttgcttactccagagattccgaaatgttagcaagtggatctcagagtggaaaggtcaaagtttggaaaatagccactggacaatgtttgaggaaactagaaaaggcacattctttaggtgttacctgtattcaattttcaagagacaacagtcaagtgttgacagcatcttttgacacatgtgtcaggatacatgggctgaaatctggaaaacttctaaaagaatttcgaggtcatacatcatttgtgaatgacatatcttttacagcagatggacataacatattgagtgcgtctagtgatggtacagtaaaaatgtggaacatcaaaacaacagaatgtacaaacacattcaagtcaattggagctagtgataaatcagttaacagtattcacatacttcctaagaataatgaacattttgttgtatgtaacaaaacaaacactgttgtcatcatgaatatgcaaggccaaattgtacgatctttgtcttctggtaaaagagaaggaggagacttcctatgttgtacaatttcccctcgtggtgaatggatctattgtgttggagaagatatggtgttatattgtttctcaattacatctggaaaattggagcgtactctcaatgttcacgagaaattagtaattggattgactcatcaccctcatcaaaatctcctaggaacctacagtgaagatggactgctccgattgtggaaaccttaa

SEQ ID NO: 15 - DNA, 100 bp, Glycaspis brimblecombei
ctcaattacatctggaaaattggagcgtactctcaatgttcacgagaaattagtaattggattgactcatcaccctcatcaaaatctcctaggaacctac

SEQ ID NO: 16 - DNA, 1107 bp, Glycaspis brimblecombei
gatctcaaacaacagatgtcacaaatttcatctactggaaccatattaagaacatctcaaaagaggtcgctttatgtaagagctttatttgattatgatcccaccaaagatgatggattaccatctcgaggattacctttccattatggagatattcttcatgtaaccaatgcaagtgatgatgaatggtggcaagctcgtcgtgttctaccttctggtgatgaacaaggaattggtattgttccttctaagaaacgttgggaaagaaaacaaagggcacgagatcgaacggtcaagtttcaaggtcatgtaccagttttattagaaaagacatcaacgttagaaagaaaaaagaagaacttctcattcagtcgaaagtttccattcatgaaaagtaaagatgataaatctgaagatggttctgaccaagaaccattcatgttatgttacacccaagacgatccaaccacagaaggtactgaagaaggtgtactgtcctatgaacctgtcactcaactgcagatagaatactcaaggcctgttattatacttggacctttgaaagatagaattaatgatgatttaatatcggagtttcctgaagagtttggatcatgtgtaccacataccaccagagctaaaagagattatgaagttgatggaagagattaccattttgttgcatcaagagaacaaatggaaaaggatattcaaaaccatctatttattgaagcaggacaatataatgataacctatatggaacttcagtggcatctgtcagagacgttgctgaaagtgggaagcattgtattttagatgttagtggaaatgccatcaaaagacttcaagtagcacagctttatcctattgcaatatttataaaaccaaaatctgttgaatctataatggaaatgaataaacgaatgactgaagagcaagcaaagaaattatatgaccgtgccatgaaaatggaacaggagtttggtgaatttttcactgctgttgttcaaggagatatgccagaagatatttaccataatgtgaaagcagtcatcaaggaacagtctggaccttcaatttgggtcccttcaaaagatcctctgtag

SEQ ID NO: 17 - DNA, 100 bp, Glycaspis brimblecombei
gaattggtattgttccttctaagaaacgttgggaaagaaaacaaagggcacgagatcgaacggtcaagtttcaaggtcatgtaccagttttattagaaaa

SEQ ID NO: 18 - DNA, 234 bp, Glycaspis brimblecombei
atggctcatacaatattgctaattcaacctggtgttaagccagagactcgaacattttcagattatgaatctgttaatgagtgtatggaaggtgtctgcaaaatttatgaggaacatttgaaaagaatgaatcccaacactccatccatcacctatgatatcagtcagttgtttgattttattgaccagttgtcagacctttcatgtctagtttatcaaaagggttccaacacg

SEQ ID NO: 19 - DNA, 48 bp, Glycaspis brimblecombei
ttgctaattcaacctggtgttaagccagagactcgaacattttcagat

SEQ ID NO: 20 - DNA, 390 bp, Glycaspis brimblecombei
tggcaaagcaacaattcgtttgcctcatgctcaactgaccaacatattcatgtttgtaaactccattctgacaaaccaatcaaaagttttgaaggccacacgaatgaagtgaatgccatcaaatgggacccccaaggaattctttggtcttcttgttctgatgatatgacattaaaaatttggtctcttgataaagatgtatgtgtccatgatctgcaagcacataataaagaaatctatactattaaatggtctccaaccgggctcgaaacagccaatcccaacatgaatttggtgctagccagtgcttcctttgactctacagtcagactgtgggatgtggaaagaggagaatgtttaaatacattgacaaggcacacaggggatagg

SEQ ID NO: 21 - DNA, 100 bp, Glycaspis brimblecombei
tgggacccccaaggaattctttggtcttcttgttctgatgatatgacattaaaaatttggtctcttgataaagatgtatgtgtccatgatctgcaagcac

SEQ ID NO: 22 - DNA, 291 bp, Glycaspis brimblecombei
attgtagaatttgctaagaggttacctggtttcgacaaattagttagggaagatcaaatttcattacttaaggcttgttcgagtgaagtaatgatgttacgaatggcaaggaggtatgatgctccttctgattcgatattgtttgcaaataaccaaccatatactagggaggcatacaagtctgccgatatgggagaaacagtagatgatctgctcaaattttgtaggcttatgtattcaatgaaagttgacaatgcagaatatgcgttgctgacagccattgttatattt

SEQ ID NO: 23 - DNA, 100 bp, Glycaspis brimblecombei
gattcgatattgtttgcaaataaccaaccatatactagggaggcatacaagtctgccgatatgggagaaacagtagatgatctgctcaaattttgtaggc

SEQ ID NO: 24 - DNA, 1389 bp, Glycaspis brimblecombei
atgggtaaagaaaagattcatattaacattgtcgttattggacatgtcgactccggcaagtctactactactggacatttgatctacaaatgtggaggtattgacaaacgtaccattgaaaagttcgagaaagaagctcaagaaatgggtaaaggatcattcaaatatgcctgggtacttgacaagctcaaggctgaacgtgaacgtggtatcaccattgatattgctctgtggaagtttgaaacagccaaatactatgtcaccattattgatgccccaggacacagagatttcatcaaaaacatgatcactggaacatctcaggctgattgtgctgtattgatcgtagctgctggtactggagaatttgaagctggtatttccaagaatggtcaaactcgtgaacatgctctccttgctttcaccttaggagtcaaacaattgattgttggagtcaacaaaatggattctactgaaccaccatacagtgagtcacgttttgaggaaatcaagaaagaagttagtggttacatcaagaaaattggttacaatccagctacagttgcatttgtacctatctcaggatggcatggagacaacatgttggaaccatctgacaagatgccatggttcaagggctgggctattgaacgtaaagaaggaaaggctgatggaaaatgtttgattgaagctttagatgcaattcttccccctagtagaccaactgaaaaacccctgcgtttaccattgcaggacgtgtacaaaattggaggtattggaacagtaccagttggtcgtgttgaaactggagtattgaaacctggtatggttgtcacctttgcccctgccaacttaaccactgaagttaaatccgtagaaatgcaccacgaagctcttcaagaagcagttccaggagacaatgttggtttcaacgtaaagaacgtctcagttaaagaattacgtcgtggatttgttgctggagattccaagtccaacccacccaaggctacccaagatttcacagcccaagtcattgtattgaaccaccctggtcaaatttcaaacggttatactcctgtacttgattgtcacacagctcacattgcttgtaaattctctgagatcaaagaaaagtgtgaccgtcgtactggtaaaactactgaagaaaatcccaaatcagtcaaatctggtgatgctgccattgtagtccttgtcccatctaaacctatgtgtgtagaatctttctctgacttccctcccttgggacgttttgctgtccgtgacatgagacaaactgttgccgtcggtgttatcaagagtgtaaattataaagatttatctgctggtaaagtaacaaaggctgctgaaaaagctgcaaagaagaaataa

SEQ ID NO: 25 - DNA, 70 bp, Glycaspis brimblecombei
atttgttgctggagattccaagtccaacccacccaaggctacccaagatttcacagcccaagtcattgta

SEQ ID NO: 26 - DNA, 1275 bp, Glycaspis brimblecombei
tctggaactttatattcgtggcccgagaacttccgcacatatcaaatccttgtaagtgcagaatactctggatttaaagtgaatatacctaaggattttgtattcggcaaatcaaacaaaagtcctgagtttgtaacgaaattttcgtcaccaaaggttccagcttttgaaggtgcagatggcactattcttacatctagtagtgccataactctgtttgtttccagtgaacaactgaggggtaaaaatgaagcagataaaatgaaagtatttgattatgtctgttttgctcaagatgaattacttcccaacgcttgcagatgggtcttccctattttagatatatacccatataataaacaaactgttgattcagcaagagatggtttgaagagaagtctctctaagcttgataaacatctcttaactcgcacctatttggttggtgattacatcactattgctgatatatgtaatgcatgtactttgttacaagtctatcaacatgctatggacccaactttcagaaaaccatatgtcaatgttaacagatggtttactactattgtcaaccaaccagaatttaagaaaattgtaggagaggtcaaattatgtgagaaacaagttaatgaagctgaacttgctagtaaaagtggtgtcaaagctcaagcaccagaagagaaaaaagagaagcccaaaaaagaaaagaaagaacaaccaaaaaaagaaaaggaagcagaacctgaggatgctggagatgccatggatgatgtattggctgctgaacctaaatcaaaggacccatttgattctatgccaaaaggcagttttgtcatggatgactataaaagattttactctaataatgatgaggcaaaatctattccttacttctgggaaaaattcgacaaagaaaactattcaatttggtttggagagtacaagtacaatgatgagcttgctaaagtttttatgagttgtaatttaattacaggaatgtttcaaagactggacaaaatgagaaagcaagcttttgcctcatgctgtctgtttggttcagataatgatagtagtatttccggaatttgggtgtggagaggacatgatcttgcctttacactatgtccagactggcaaattgattatgaatcttatgattggaaaaaattagatccagaagcaaaagaaacaaaagatttggtcacccaatacttttcatggacaggcactgattctaagggtcgtaaatttaatcaaggaaaaatctttaagtga

SEQ ID NO: 27 - DNA, 100 bp, Glycaspis brimblecombei
caactttcagaaaaccatatgtcaatgttaacagatggtttactactattgtcaaccaaccagaatttaagaaaattgtaggagaggtcaaattatgtga

SEQ ID NO: 28 - DNA, 690 bp, Glycaspis brimblecombei
aacaaattaattagagtaggaaaaacagaacctgttgttgttatcagagttgacaaagaaaaaggttatattgatttaagtaaaagaagagtatcaccagaagatgtagaaaaatgtactgaaagatatgctaaggctaaagcagttcattctatcttgaggcatgttgctgaaatccttcattttgattcagacaaacagttggaagatctttatcaaagaactgcatggaattatgaagataaaacaaaaaagaaaggttcttcatatgatttcttcaaacaagctgtcctagatcccaatacattgatagaatgtggtcttgatgaacatacaagagatgtcctagtaaacaatattcaacgtaaacttacatcccaagctgtaaagatcagagctgatattgaagtagcatgttatggttatgaaggtattgatgctgttaagacagccttaaaagctggtttagcaatgtccacggagaaattacccattaaaatcaatcttattgctcctccattgtatgtaatgacaacagtaacaccagaaaaagctgatggattaaaagcactccaagaagcaatcgacaccattaaaataaaaattgaagaactaggtggtgtgttccaagttcaaatggcgcccaaagtggttacagcaagtgacgaagctgaattggctcgtcaaatg

SEQ ID NO: 29 - DNA, 100 bp, Glycaspis brimblecombei
tatgctaaggctaaagcagttcattctatcttgaggcatgttgctgaaatccttcattttgattcagacaaacagttggaagatctttatcaaagaactg

SEQ ID NO: 30 - DNA, 897 bp, Glycaspis brimblecombei
cgagaccttatcttgatgtctcaccttcaggaaactattcaatactctgatccttcaacacaaatcttgtacaataggacgatggctcacctcggtttgtgtgcattccgtcacgcgcacatcaaagatgctcataattgtctggttgatttaatgatgactggaaaagtgaaggagttgcttgcccagggtcttatgccccaacgacaacatgagcgtagcaaagaacaggaaaaagttgaaaaacagcgtcagattccattccatatgcacatcaacctagagctgcttgagtgtgtttatttggtgtcagctatgctcatagaaataccctacatggctgctcatgagttcgatgcccgccggaggatgatttctaaaactttctatcaacaacttcgttccagtgaacgtcaaagtctggtaggaccccctgaatcgatgagagagcatgtagtagccgccagtaaagctatgagacaaggaaattggaaaaattgtgtcaattttataataaatgaaaaaatgaacgctaaagtttgggatttgttttatgagtcgagtaaaactcgttctatgctgactcgtcttatcaaagaagaatctttgagaacttatctgttcacattctctcatgtgtattcatcaatttctatgaataccttgtcggcaatgtttgaaatggaaaagcttagcgtacattctatcatctctaaaatgataattaatgaagaattgatggcatctcttgatgatccaacccaaacagtggtccttcatcgatctgaaccatctagacttcaagcgctagcacttcaattggcagacaaagttaataacttcgttgactcaaatgaacgtatctttgaaatgaagcaaggcaatttcttccaaaga

SEQ ID NO: 31 - DNA, 100 bp, Glycaspis brimblecombei
cccaaacagtggtccttcatcgatctgaaccatctagacttcaagcgctagcacttcaattggcagacaaagttaataacttcgttgactcaaatgaacg

SEQ ID NO: 32 - DNA, 100 bp, Glycaspis brimblecombei
cccaaacagtggtccttcatcgatctgaaccacctagacttcaagcgctagcacttcaattggcagacaaagttaataacttcgttgactcaaatgaacg

SEQ ID NO: 33 - DNA, 1119 bp, Glycaspis brimblecombei
aatgttactgatgcattttatcgttataaaatgccaaagcttatagctaaggtagagggtaaaggcaatggaattaagactgtcattgtcaatatggtagatgtggcaaaagcactgggacgtcctccaacttaccccactaaatattttggttgtgagttgggtgcacaaacaaaacttgaccataaaaatgatcgctacattgttaatggttcccatgatgttacaaagcttcaggacttgcttgatggattcatcagaaaatttgttctttgtcctgagtgtgacaatccagagacagatctaattgtttcagcaaagaagcaaaccattcagcaaggttgcaaggcatgtggacatcatggcctgctcactttcaaccacaagttgaatactttcattttaaagaatcctcccaacttgaatcctgctgtgcaaggatcatcattgactgagggaaagcgtcctaaacgtgaaagtaagaagcaagatgctaatggtgacatctctaaatcagatgaggaaggtgactggccagtacaagctccagagaagattggtgataatgaggatgattgtgactggactgaagatgtgagtgaagaagctgtaagagctcgtatgcaagatttgaccacaggagttaaaggtttaacaattactgatgatttagataaaactgaaaaagaacggatggatatattttattcatgtgtaaaagcagctcttgagaaaaataatctggatgctaaggaaatcctgactgaagctgaacgcttggaagtgaaaactaaagcaccccttgttctagctgaactgctttttgatgataaaattcacattcagatgaaaaaacaccgcattttattgttgcgtttcacccatgaagatactaaagcccaacgttatctcttaaatggaatagaacaagtcatagctttgcataaagatgtacttttagcaaaagtaccagctatactgaaacttttctatgatgctgatattttggaggaaaaagtattgctagaatgggctgaaaaggtttccaaaaaatatgtctctaaagagctgagtgcagagattcgttctcgtgctgaaccatttattaaatggttacgt

SEQ ID NO: 34 - DNA, 100 bp, Glycaspis brimblecombei
tttattcatgtgtaaaagcagctcttgagaaaaataatctggatgctaaggaaatcctgactgaagctgaacgcttggaagtgaaaactaaagcacccct

SEQ ID NO: 35 - DNA, 354 bp, Glycaspis brimblecombei
tttcttatagctatagcagaacctgtgtgtcgtccttttacacgattcactgaatacaagttaacagcatattctttatatgctgcggtcagtgtcggattacagactcatgatattattgaatatcttaaaagactgagtaaaacatccgtgcctgatggtatagtagagtttatcacactttgtacattatcttatggaaaggttaaattagtgctaaaacacaatagatatttcatagaatcacaatttgcagatgttttacaaaaacttttaaaagatcctgtgatacaagaatgtcgtctaagacgtgatgttgaagattcacaaactcttatcagtgaaactgataag

SEQ ID NO: 36 - DNA, 69 bp, Glycaspis brimblecombei
gtgatacaagaatgtcgtctaagacgtgatgttgaagattcacaaactcttatcagtgaaactgataag

SEQ ID NO: 37 - DNA, 654 bp, Glycaspis brimblecombei
gtgcaagaaatattccgaaatactcctcatcaaaaacaagttatgatgttttcagccactttgagcaaagaaatccgtccagtgtgcaaaaagtttatgcatagatccaatggaggtttatgtggatgatgatgccaaacttactctgcatggacttcaacaacattacgtcaaactgaaggagaatgaaaaaaataaaaactttttgaactacttgatgccctagatttcaatcaagttgtggtatttgtaaagtcggtcactcgttgtattgctctatcatcgctcctatcggaacagaattttcctgctactggcatccaccgtggtatgactcaagaagaaagacttaaaaaataccaagaattcaaagatttccaaaagagaatccttgtggccaccaacttatttggtcgtggtatggacattgagaaggttaacattgtattcaactatgacatgcctgaagattctgacacttatctacacagagtggcacgtgcagggcgattcggcacaaagggtttagccatcacctttgtttgtgatgaaaatgatgctaaaattttaaacaacgtacaagagagatttgatgtgagcattactgtactacctgatgaaattgacttgtcgacctatattgaaggacgataa

SEQ ID NO: 38 - DNA, 100 bp, Glycaspis brimblecombei
gcctgaagattctgacacttatctacacagagtggcacgtgcagggcgattcggcacaaagggtttagccatcacctttgtttgtgatgaaaatgatgct

SEQ ID NO: 39 - DNA, 291 bp, Glycaspis brimblecombei; identical to SEQ ID NO: 22

SEQ ID NO: 40 - DNA, 100 bp, Glycaspis brimblecombei
ggtatgatgctccttctgattcgatattgtttgcaaataaccaaccatatactagggaggcatacaagtctgccgatatgggagaaacagtagatgatct

SEQ ID NO: 41 - DNA, 915 bp, Glycaspis brimblecombei
gaattgctgctcaagcgaggtgccaataaagaacatagaaatgtatcggattatactccgctaagcctagctgcaagtggaggatatgtgaatataatcaaactgctgcttacacatggcgctgagataaactcccgcactggatctaagctaggaatatcacctcttatgttagcagctatgaatggccacactccagctgtaaaactcttgctggatatgggaagtgatattaatgctcagattgaaaccaataggaatacagcacttactctagcatgcttccaaggaaggcatgaagttgtgagtttgctgttggacaggaaagctaacgtagagcatagagcaaagactggacttacgcccttgatggaagctgcaagtgggggctacaccgatgttgggcgcgttctattagataaaggtgctgatgtaaatgcccctcctgtgccttcatctcgagatactgcattaactattgctgctgataaaggtcatggcagattcgtagaccttttattgtccagaggagcccaagtagaagttaaaaataaaaaaggaaactctcccctatggttggctgccaatggtggccatcagagtgttgtggcactactttggaaacatcgtgcagatattgattctcaagacaaccgtcaagtttcatgtttgatggctgcattccgtaaaggtcactgcaaagtggttcagtggatggttaatcatgttgctcaatttcctagtgatcaggaaatgacgcgatatatacaaactgtcaatgataaggaccttctaaataaatgtcaagaatgtttgatgtcaattagagctgcaaaaaatcaacaagctgagaaagctaacaaaaatgctaatatacttttagaagaactagatatggaaaagtggcgggaagaa

SEQ ID NO: 42 - DNA, 100 bp, Glycaspis brimblecombei
gctgagataaactcccgcactggatctaagctaggaatatcacctcttatgttagcagctatgaatggccacactccagctgtaaaactcttgctggata

SEQ ID NO: 43 - DNA, 834 bp, Glycaspis brimblecombei
agattgaatccgatggaatatgtcactagtacagcttgtaggcgaaatttagcaggggatgtgtgtgctataatgcgagttcatgctttcttagaacagtggggattaattaattaccaggttgatagcgattcaaaaccatctgctattggtccaccacctacatctcacttccatgttttaactgacactccatctggacttcaacctgttaatcctcctaaaacaacacaaccctcagctgctaagatattgttggatatggataagaaaccagatacgctactcaagaaagaaggctctgagatcccatctaattttggattgaaattagaccagtatgctaagaagccagcagttttgagaaacaaacaagctgctagcatggttcgagattggacagaacaagaaactttgctcttgctggaagctctagaaatgtacaaagatgattggaataaagtttgtgaacatgttggaagtcgaactcaggatgagtgcattttacatttcttaagattgccaattgaagacccatatttagaagatcctgagtctggtggaggtgcattaggtcctttagcttatcaaccaataccattcagcaaggctggtaatcccatcatgtcaactgtagcctttttagcatcagttgttgatccccgagttgcttcttctgctgcaaaagctgccatggaagaatttgcacgtattaaggatgaagtcccagctgctattatggatgcacacatcaagaatgttgaagcctccaccgcagacggaaaatatgatcctgctgcaggacttttgcagagtggaatagcaggaactgtt

SEQ ID NO: 44 - DNA, 100 bp, Glycaspis brimblecombei
agatattgttggatatggataagaaaccagatacgctactcaagaaagaaggctctgagatcccatctaattttggattgaaattagaccagtatgctaa

SEQ ID NO: 45 - DNA, 678 bp, Glycaspis brimblecombei
aaggaagctgaaaaggaatgggcacctgtcaccaaattgggtcgcttggttagagatggtaaaattcaatcattagaacaaatttacttgttctctctacccatcaaggaatttgaaatcattgacttctttattggatcagtcttaaaagatgaggtactcaaaatcatgcctgtacagaaacaaaccagagctggtcaaagaactagattcaaggcttttgttgccattggtgactctaatggacatattggtttaggtgttaagtgctctaaggaagtagcaactgccatccgtggagctatcatcttagctaaattgtctgttgttccagtcagaagaggttactggggaaacaagattggtaaaccccacaccgtaccttgcaaggttactggtaaatgtggttcagttcaagtacgtctcatccctgctcctcgtggtacaggtattgtaggagctccagtccctaagaaattattgcagatggctggtattgaagattgttatacctcagctagaggttcaacttgtactcttggtaacttcgccaaagctacatatgctgctattgccaagacatatgcttacctgacaccagatttatggaaagacaacccacttagaaaagccccttacagtgaattcagtgagttcttggaaaagaatcatcgcatt

SEQ ID NO: 46 - DNA, 100 bp, Glycaspis brimblecombei
gacatatgcttacctgacaccagatttatggaaagacaacccacttagaaaagccccttacagtgaattcagtgagttcttggaaaagaatcatcgcatt

SEQ ID NO: 47 - DNA, 663 bp, Glycaspis brimblecombei
atggccgaagattgggatactgatccagcttatcctgaaatagccactggcccagtaggattatcttcaattgctgctcctgctgaattaccagaaattaaattatttggaagatggagttgtgatgatgtccaagttagtgatatgtccctccaggattatattgctgttaaagaaaagaatgcaaaatatttacctcactcagctggaagatatgctgccaaaagattccgtaaagcacaatgtcccattgtagaaagattaactaactcacttatgatgcacggacgtaacaatgctaaaaaattaatggctgttagaattgttaaacacgcttttgaaattattcatttgttaactggagagaatcccctccaagtcttggtaactgccatcatcaattcaggaccaagagaagattcaacacgtattggacgtgccggtacagtaagaagacaagctgttgatgtatcacctttaagaagagtaaatcaagctatctggttattgtgtactggtgccagagaagctgccttcagaaatattaaaactattgctgaatgtgttgctgatgaacttatcaatgctgctaagggatcatctaactcctatgctattaagaagaaagatgaattggaacgtgtcgccaagtccaatcgttaa

SEQ ID NO: 48 - DNA, 100 bp, Glycaspis brimblecombei
atggccgaagattgggatactgatccagcttatcctgaaatagccactggcccagtaggattatcttcaattgctgctcctgctgaattaccagaaatta

SEQ ID NO: 49 - DNA, 993 bp, Glycaspis brimblecombei
atgaaaccattaattcttcaaggtcatgaaagatcaatcacccaaattaaatacaacagagaaggtgatatcttaatcagttgtgccaaggatgctgatgcaaatgtatggtattcagttaatggtgagagagccggaactcttagtggtagtaaaggtaccatttggacaattgatattgactggatgactacaagagtactgactggtcatgctgatggaaaacttaaaatgtgggatatttcaaatggaacaacaatcagtgatattccaacattctcaaattgtgctgtcagaacatgtgggtttagttactcatcaaatttatgtgcttacagtaatgatgacaccatgggaaataaatgttatctctccgttcttgatgttaggactactgatgctacaaattcaggagacccagttgttaagatgcaagtgacagatgaatctgccaaaattacatctatgttgtggggtaacttagatgaatacatcatcacaggccatgccaaaggtgatatttgcacctgggatataagaatgggaagacatttagaaagtgttaacgctcatcctggacaaccaattaatgatatgcaattttccaaggactctacaatgtttattacagcctccaaagatcatactgctaaattatttagttctggagattgtcaacttttgaaaacatacaccactgaaagacctgtcaacagtgctgctttatcaccaatcctacctcatgttgtacttggaggaggtcaagaagctcgtgaagtaacaacaacttcgactaaagtcggaaaattcgatgctagattttatcatttaatatttgaagaagaatttgcacgtattaaaggtcattttggtcctattaactctttagcttttcatccagatggaaaatcatatgctagtggtggagaagacggttttgttaggttacatacatttgatcaatcttatttcgattatacttttgacatttag

SEQ ID NO: 50 - DNA, 100 bp, Glycaspis brimblecombei
ttacagtaatgatgacaccatgggaaataaatgttatctctccgttcttgatgttaggactactgatgctacaaattcaggagacccagttgttaagatg

SEQ ID NO: 51 - DNA, 456 bp, Glycaspis brimblecombei
tctggacggtatatgatctcaagagattacctttgtgttaaagtttgggatttacatatggagtctagaccagtagaaacttacccagttcatgaataccttagatcaaaattatgttcactatatgaaaatgactgtatatttgacaagtttgaatgttgttggtccggtaacgattcagcaattatgacaggctcttataataatttcttccgtatgtttgatagaattaataaacgagatgccacactagaggcatcaagggaaatagcaaagcctaaaacactacttagacctagaaaagtatgtacagctggtaaaaggaaaaaagatgaaatcagtgtagactgtttggacttcaataagaaaatccttcatacggcctggcatcctagtgaaaatataattgctgtagcggcaactaacaatttatatttgttccatgataagttgtag

SEQ ID NO: 52 - DNA, 100 bp, Glycaspis brimblecombei
tcttccgtatgtttgatagaattaataaacgagatgccacactagaggcatcaagggaaatagcaaagcctaaaacactacttagacctagaaaagtatg

SEQ ID NO: 53 - DNA, 354 bp, Glycaspis brimblecombei
atgtcttcaccaagtgcgggaaagcgacggatggatacggatgtcataaaactaattgaaagtaaacatgaagtcaccattttgggaggactaaatgaattctgtgttaaatttttcggacccagagatacgccatatgaaggaggagtttggaaagttagagtacatcttccagaacactaccctttcaaatcaccctctattgggttcatgaataaagtatatcaccctaatatagatgaagtctcaggtacggtgtgtctcgacgtcattaaccaagcgtggacggcgctatatgatctttcaaatatttttgtatctttcttacctcaactgttaacctaccccaaccct

SEQ ID NO: 54 - DNA, 95 bp, Glycaspis brimblecombei
aattctgtgttaaatttttcggacccagagatacgccatatgaaggaggagtttggaaagttagagtacatcttccagaacactaccctttcaaa

SEQ ID NO: 55 - DNA, 423 bp, Glycaspis brimblecombei
atggcaggagtggttgtgcctagaaattttcgtttacttgaagaattagagcaaggtcaacgaggtgttggagatggaacaatcagttggggtcttgaaaatgatgatgatatgactttgacacattggactggaatgattataggaccacctaggacaccatatgaaaatagaatgtacagtttaaggattgaatgtggaccgagatatcccgacgaaccaccaagtgcccgattcatatcaagaatcaacatgaactgtataaatagtaattctggaattgtggatcaaaaaaatgtaccagttctagctagatggcaacgagagtatactatcaagtctttattacaagaactgcgaagattaatgactgtaaaagataatactaaactctcacaaccacctgaagggagcacattttaa

SEQ ID NO: 56 - DNA, 100 bp, Glycaspis brimblecombei
agagtatactatcaagtctttattacaagaactgcgaagattaatgactgtaaaagataatactaaactctcacaaccacctgaagggagcacattttaa

SEQ ID NO: 57 - DNA, 914 bp, Artificial Sequence
  source: /note=“Synthetic polynucleotide”
agattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatatttttacaacaattaccaacaacaacaaacaacaaacaacattacaattactatttacaattaca

SEQ ID NO: 58 - DNA, 301 bp, Figwort mosaic virus
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttcaaaaaacgcagctataaaagaagccctccagcttcaaagttttcatcaacacaaattctaaaaacaaaattttttagagagggggagtg

SEQ ID NO: 59 - DNA, 921 bp, Arabidopsis thaliana
gtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttgtcttcacatataaatattgtttgaatgtcttcaatcttttatttgtatgagacaattatttaagtatcgggtgacaatgcagctattatgtattgtcgattgttatattggcgcccaaaatatatacttagcctaagaatttggtaagtgagtggcttatgttttactccagcaaaaattgtgtgtgtattaccattctgatgcgaaacaagaaaagaatttgatctaagaaaccaagtttattcactagttaaaaaacaaatgacctaatgtaatcgactccacatatcaaaatacgtaaaacaaacattgtatgttgacaaaagggaaaagaaatgatttatttggttaaaaagaaagctggattcaattgcaacagtttagtcgaaatcattttgaaaggcttacaatggattgaatgtgaatattccattaagccgcttctgtctacacagaatgttacgcttggagagcagcaatcattttcacgtttttatctttttaggtggacatgtatattattggttacgcctttggagtttttcgaaatttatttctttcaaatcacaagatgactaaacatcacaatctgtttatcttcctaactagttaaatttttgtccccaccatt

SEQ ID NO: 60 - DNA, 253 bp, Artificial Sequence
  source: /note=“Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 61 - DNA, 106 bp, Artificial Sequence
  source: /note=“Synthetic polynucleotide”
ggctcgaacgagccgactaattgtctttaaacgcgcgatataagcgcacaatgctcgagaaacgataaactctatcgctctgtcgcgtgcgtggcatcttcgcgcg

SEQ ID NO: 62 - DNA, 706 bp, Artificial Sequence
  source: /note=“Synthetic polynucleotide”
agctttagaccgtctggttaccaaaaaagctggtttctctacttctcacatcatctgtggccaaacataccctagaaaagttgacgtcatcgtaacgggactcaattacatctggaaaattggagcgtactctcaatgttcacgagaaattagtaattggattgactcatcaccctcatcaaaatctcctaggaacctaccaactttcagaaaaccatatgtcaatgttaacagatggtttactactattgtcaaccaaccagaatttaagaaaattgtaggagaggtcaaattatgtgagcgcgcgaaacaacggtaatcaaccggcaattattaatcgtacatgcgcggcgcactcgagtgcattatccctcgtcatcaccaaagcgccacattatgcttcttctcacataatttgacctctcctacaattttcttaaattctggttggttgacaatagtagtaaaccatctgttaacattgacatatggttttctgaaagttggtaggttcctaggagattttgatgagggtgatgagtcaatccaattactaatttctcgtgaacattgagagtacgctccaattttccagatgtaattgagtcccgttacgatgacgtcaacttttctagggtatgtttggccacagatgatgtgagaagtagagaaaccagcttttttggtaaccagacggtctaaagct

SEQ ID NO: 63 - DNA, 300 bp, Artificial Sequence
  source: /note=“Synthetic polynucleotide”
agctttagaccgtctggttaccaaaaaagctggtttctctacttctcacatcatctgtggccaaacataccctagaaaagttgacgtcatcgtaacgggactcaattacatctggaaaattggagcgtactctcaatgttcacgagaaattagtaattggattgactcatcaccctcatcaaaatctcctaggaacctaccaactttcagaaaaccatatgtcaatgttaacagatggtttactactattgtcaaccaaccagaatttaagaaaattgtaggagaggtcaaattatgtga

SEQ ID NO: 64 - DNA, 706 bp, Artificial Sequence
  source: /note=“Synthetic polynucleotide”
cccaaacagtggtccttcatcgatctgaaccacctagacttcaagcgctagcacttcaattggcagacaaagttaataacttcgttgactcaaatgaacggcctgaagattctgacacttatctacacagagtggcacgtgcagggcgattcggcacaaagggtttagccatcacctttgtttgtgatgaaaatgatgctagagtatactatcaagtctttattacaagaactgcgaagattaatgactgtaaaagataatactaaactctcacaaccacctgaagggagcacattttaagcgcgcgaaacaacggtaatcaaccggcaattattaatcgtacatgcgcggcgcactcgagtgcattatccctcgtcatcaccaaagcgccacattatgcttcttcttaaaatgtgctcccttcaggtggttgtgagagtttagtattatcttttacagtcattaatcttcgcagttcttgtaataaagacttgatagtatactctagcatcattttcatcacaaacaaaggtgatggctaaaccctttgtgccgaatcgccctgcacgtgccactctgtgtagataagtgtcagaatcttcaggccgttcatttgagtcaacgaagttattaactttgtctgccaattgaagtgctagcgcttgaagtctaggtggttcagatcgatgaaggaccactgtttggg

SEQ ID NO: 65 - DNA, 300 bp, Artificial Sequence
  source: /note=“Synthetic polynucleotide”
cccaaacagtggtccttcatcgatctgaaccacctagacttcaagcgctagcacttcaattggcagacaaagttaataacttcgttgactcaaatgaacggcctgaagattctgacacttatctacacagagtggcacgtgcagggcgattcggcacaaagggtttagccatcacctttgtttgtgatgaaaatgatgctagagtatactatcaagtctttattacaagaactgcgaagattaatgactgtaaaagataatactaaactctcacaaccacctgaagggagcacattttaa

SEQ ID NO: 66 - DNA, 706 bp, Artificial Sequence
  source: /note=“Synthetic polynucleotide”
agatattgttggatatggataagaaaccagatacgctactcaagaaagaaggctctgagatcccatctaattttggattgaaattagaccagtatgctaattacagtaatgatgacaccatgggaaataaatgttatctctccgttcttgatgttaggactactgatgctacaaattcaggagacccagttgttaagatgtcttccgtatgtttgatagaattaataaacgagatgccacactagaggcatcaagggaaatagcaaagcctaaaacactacttagacctagaaaagtatggcgcgcgaaacaacggtaatcaaccggcaattattaatcgtacatgcgcggcgcactcgagtgcattatccctcgtcatcaccaaagcgccacattatgcttcttccatacttttctaggtctaagtagtgttttaggctttgctatttcccttgatgcctctagtgtggcatctcgtttattaattctatcaaacatacggaagacatcttaacaactgggtctcctgaatttgtagcatcagtagtcctaacatcaagaacggagagataacatttatttcccatggtgtcatcattactgtaattagcatactggtctaatttcaatccaaaattagatgggatctcagagccttctttcttgagtagcgtatctggtttcttatccatatccaacaatatct

SEQ ID NO: 67 - DNA, 300 bp, Artificial Sequence
  source: /note=“Synthetic polynucleotide”
agatattgttggatatggataagaaaccagatacgctactcaagaaagaaggctctgagatcccatctaattttggattgaaattagaccagtatgctaattacagtaatgatgacaccatgggaaataaatgttatctctccgttcttgatgttaggactactgatgctacaaattcaggagacccagttgttaagatgtcttccgtatgtttgatagaattaataaacgagatgccacactagaggcatcaagggaaatagcaaagcctaaaacactacttagacctagaaaagtatg

SEQ ID NO: 68 - DNA, 306 bp, Artificial Sequence
  source: /note=“Synthetic polynucleotide”
tcttccgtatgtttgatagaattaataaacgagatgccacactagaggcatcaagggaaatagcaaagcctaaaacactacttagacctagaaaagtatggcgcgcgaaacaacggtaatcaaccggcaattattaatcgtacatgcgcggcgcactcgagtgcattatccctcgtcatcaccaaagcgccacattatgcttcttccatacttttctaggtctaagtagtgttttaggctttgctatttcccttgatgcctctagtgtggcatctcgtttattaattctatcaaacatacggaaga

SEQ ID NO: 69 - DNA, 506 bp, Artificial Sequence
  source: /note=“Synthetic polynucleotide”
ttacagtaatgatgacaccatgggaaataaatgttatctctccgttcttgatgttaggactactgatgctacaaattcaggagacccagttgttaagatgtcttccgtatgtttgatagaattaataaacgagatgccacactagaggcatcaagggaaatagcaaagcctaaaacactacttagacctagaaaagtatggcgcgcgaaacaacggtaatcaaccggcaattattaatcgtacatgcgcggcgcactcgagtgcattatccctcgtcatcaccaaagcgccacattatgcttcttccatacttttctaggtctaagtagtgttttaggctttgctatttcccttgatgcctctagtgtggcatctcgtttattaattctatcaaacatacggaagacatcttaacaactgggtctcctgaatttgtagcatcagtagtcctaacatcaagaacggagagataacatttatttcccatggtgtcatcattactgtaa

SEQ ID NO: 70 - DNA, 200 bp, Artificial Sequence
  source: /note=“Synthetic polynucleotide”
ttacagtaatgatgacaccatgggaaataaatgttatctctccgttcttgatgttaggactactgatgctacaaattcaggagacccagttgttaagatgtcttccgtatgtttgatagaattaataaacgagatgccacactagaggcatcaagggaaatagcaaagcctaaaacactacttagacctagaaaagtatg

SEQ ID NO: 71 - DNA, 666 bp, Glycaspis brimblecombei
atgctggcacttcgccctgcagttttggcaagagcctccaatgtatgtgtaagaagtctttcttctcctccatctacaagtaccaaatcaggatcttctgttgaagatgtcactaaaaactctgctattgaagctgaaacaagagctcctgttagaagagaggattacagtcctttcaatgttacaagaaaagataacatgtttgagtacactctagcaagacttgatgatgttctcaactggggaagaaaaaattctatttggcctctaactttcggtctagcatgttgtgctgtagaaatgatgcacattgctgctcctagatatgatatggacaggtatggtgtagtcttccgtgcctctcctagacaagctgatgttattattgtagctggaaccttaaccaataaaatggcgcctgctcttagaaaagtttatgaccagatgttggatccacgttgggtaatttcaatgggtagttgtgcaaatggtggtggatattaccattattcttattcagttgttcgaggatgtgatcgtattattcctgtagatatttatgtaccaggatgtccaccaactgctgaggcacttatgtatggtatcttacaattacaaaagaaagttaagagaatgagaactttccagatgtggtacagacgataa

SEQ ID NO: 72 - DNA, 100 bp, Glycaspis brimblecombei
acagtcctttcaatgttacaagaaaagataacatgtttgagtacactctagcaagacttgatgatgttctcaactggggaagaaaaaattctatttggcc

SEQ ID NO: 73 - DNA, 336 bp, Glycaspis brimblecombei
gatcgtcctattacaatcaaagatgataaaggaaatacatccaaatgtattgctgatattgtttcattatttattaccatcatggacaaattacgtctagaaataaaagctatggatgagttacatcctgatctacgtgatctcatggatactatgaacagattgagtattttgccctccaactttgaaggaaaggaaaaggtttctaattggttgaatgttttgacagctatgtcagccagtgatgagctcaatgaaacacaagtgagacagttgctatttgatttagaaacatcttacaatgcattcaataagatccttcatcaatctgcttga

SEQ ID NO: 74 - DNA, 100 bp, Glycaspis brimblecombei
gtattttgccctccaactttgaaggaaaggaaaaggtttctaattggttgaatgttttgacagctatgtcagccagtgatgagctcaatgaaacacaagt

SEQ ID NO: 75 - DNA, 294 bp, Glycaspis brimblecombei
gcaaatattcaagcagtatctttgaagatccaaactcttagatcacagaatgcaatggcagaagctatgaaaggttgttctagagctatggcaaacatgaataggcaaatgaatttaccacaaattcagcgaatattatcagaatttgagaaacaatcagaaataatggacatgaaagaagaaatgatgaatgatgcaatggatgatgccatgggagatgatgatgatgaagaggaaacggatgcagttgttacacaagttcttgatgaattaggtctccaattaaatgaccaa

SEQ ID NO: 76 - DNA, 100 bp, Glycaspis brimblecombei
tggcagaagctatgaaaggttgttctagagctatggcaaacatgaataggcaaatgaatttaccacaaattcagcgaatattatcagaatttgagaaaca

SEQ ID NO: 77 - DNA, 417 bp, Glycaspis brimblecombei
ggtgataaagatgtctgtgttacgttggccaaggaaattatcaatgcaaggaaacatataacaaagattcatacatcaaaagcccatctgaattctatacaattgcaaatgaaaaatcaattatctttattaagagtatctggatcaatacaaaaatcaacagaagttatgcaagctatgcagaatctagtaaatgtacctgaagtagcaaacacaatgagagaaatgtccaaagaaatgatgaaagctggaattatggaagagatgattgaagaaactatggaatccttagaaccagaggacactgaagatatggaagaagaagcacaaaaagaaattgataaggtactctgggacttaacagctggggcacttggtaaagtaccggatgctgttaaagatgtaccatcctcatct

SEQ ID NO: 78 - DNA, 100 bp, Glycaspis brimblecombei
ggtgataaagatgtctgtgttacgttggccaaggaaattatcaatgcaaggaaacatataacaaagattcatacatcaaaagcccatctgaattctatac

SEQ ID NO: 79 - DNA, 408 bp, Glycaspis brimblecombei
cagagagaagctctggaaggtgccaatactaatactgcagttctaacaacaatgaagaatgctgcagatgctcctaaagctgcacacaaacacatggatgttaaccaagtacacgatatgatggatgatattgctgaacagcaagatgtagccaaggaaatatctgaagccatttctaatccagttgcctttggtcatgatgtagatgaggatgagttagaaaaagaattagaagaattagaacaagaagaattggataaggatctgcttaaactaagtacgcctggtgatgatctacctgaactaccatccactgcaccaaaagacaaagccaaaagaaaaagctaggcacaaaggaacgttctagtagatgatgaaatcaaagaattagaagcatgggcttcataa

SEQ ID NO: 80 - DNA, 100 bp, Glycaspis brimblecombei
ggatctgcttaaactaagtacgcctggtgatgatctacctgaactaccatccactgcaccaaaagacaaagccaaaagaaaaagctaggcacaaaggaac

(19) United States
(12) Patent Application Publication
(10) Pub. No.: US 2018/0037907 A1
(43) Pub. Date: Feb. 8, 2018

(54) Glycaspis Brimblecombei Control Agents

(71) Applicant: Futuragene Israel Ltd., Rehovot (IL)

(72) Inventors: Dror Avisar, Kochav Yair (IL); Daniel Siegel, Rehovot (IL); Ziv Shani, Mazkeret Batia (IL)

(21) Appl. No.: 15/681,187

(22) Filed: Aug. 18, 2017

Related U.S. Application Data
(63) Continuation of application No. 14/396,585, filed on Oct. 23, 2014.
(60) Provisional application No. 61/787,354, filed on Mar. 15, 2013.
(60) Provisional application No. 61/636,940, filed on Apr. 23, 2012.

(51) Int. Cl. C12N 15/82; C12N 15/113

(52) U.S. Cl. CPC C12N 15/8286; C12N 15/8218; C12N 15/113; C12N 2310/531; C12N 2330/50; C12N 2310/14

(57) ABSTRACT

The present invention relates to the field of RNA-mediated gene silencing in insect species. The present invention is based, in part, on the inventors' sequencing of genes from eucalyptus invasive species Gb pest, Glycaspis brimblecombei. In certain aspects, the invention provides Gb nucleic acids, derivatives thereof and the use of such nucleic acids and derivatives as Gb control agents.

### SEQUENCE LISTING

The instant application contains a Sequence Listing which has been submitted in ASCII format via electronic filing and is hereby incorporated by reference in its entirety. Said ASCII copy, created on Apr. 18, 2013, is named 30407-0004WO1_SL.txt and is 50,196 bytes in size.

### FIELD OF THE INVENTION

The present invention relates to the field of double stranded RNA (dsRNA)-mediated gene silencing in insect species.

### BACKGROUND

The red gum lerp psyllid, Glycaspis brimblecombei (Gb) is a sap-sucking pest (Order Hemiptera: Psyllidae) exclusively found on eucalyptus trees. Gb infestations have occurred in many countries and pose a threat to natural populations and commercial eucalyptus farming in Africa, South and North America, India, Australia and the Mediterranean. Eucalyptus species differ in their susceptibility to attack by Gb. E. camaldulensis and E. tereticornis are highly susceptible whereas E. grandis is more tolerant. Gb is an aggressive pest that spreads rapidly. Symptoms of Gb infestation include leaf loss and drying of lead shoots. Severe infestation can cause complete defoliation and death of trees.

Gb females lay between 45 and 700 eggs per lifetime. Eggs hatch within 10 to 20 days and the emerging nymphs pierce eucalyptic tissue with their stylet (mouthparts), feeding on the xylem and phloem. As the nymphs feed on plant sugars from the leaves they secrete honeydew with which they construct a waxy protective cover (“lerp”) around themselves. The lerp is whitish and conical in shape and shelters insects during development, until they reach adult stage. In Australia there are typically two to four Gb generations per year.

Efforts to control Gb infection of eucalyptus have included attempts to isolate naturally resistant plants and natural predators. Such efforts, however, have met with limited or no success.

Chemical pesticide control of Gb is costly and environmentally unfriendly. Chemical pesticides are potentially detrimental to the environment, are not selective and are potentially harmful to non-target crops and fauna. Chemical pesticides persist in the environment and generally are metabolized slowly, or not at all. Chemical pesticides accumulate in the food chain, particularly in the higher predator species where they can act as mutagens and/or carcinogens to cause irreversible and deleterious genetic modifications. Crop pests, moreover, may develop resistance against chemical insecticides because of repetitive usage of the same insecticide or of insecticides having the same mode of action.

RNA interference or “RNAi” is a process of sequence-specific down-regulation of gene expression (also referred to as “gene silencing” or “RNA-mediated gene silencing”) initiated by double-stranded RNA (dsRNA) that is complementary in sequence to a region of the target gene to be down-regulated. Down-regulation of target genes in multicellular organisms by means of RNA interference (RNAi) has become a well-established technique. U.S. patent application publications US 2009/0285784 A1 and US 2009/0298787 relate to dsRNA as an insect control agent and are hereby incorporated herein by reference in their respective entireties. U.S. Pat. No. 6,506,559, U.S. patent application publication 2003/00150017 A1, International Publications WO 00/01846, WO 01/37654, WO 2005/019408, WO 2005/049841, WO 05/047300 relate to the use of RNAi to protect plants against insects. International application, PCT/US12/31423, filed Mar. 30, 2012, relates to RNA-mediated control of eucalyptus pests in the Gall Wasp family. Each of the foregoing patents and published applications is hereby incorporated by reference in its entirety.

### SUMMARY

The present invention is based, in part, on the inventors' sequencing of genes from the eucalyptus red gum lerp psyllid invasive species, Glycaspis brimblecombei (Gb). In certain aspects, the invention thus provides Gb nucleic acids, derivatives thereof and the use of such nucleic acids and derivatives as Gb control agents.

In certain aspects the invention provides isolated nucleic acids that hybridize selectively under high stringency hybridization conditions to a sequence set out in SEQ ID NO: 1-56 and 71-80 and complementary sequences thereof.

In certain aspects the invention provides isolated nucleic acids that are 90-99.99 percent identical to sequences set out in SEQ ID NO: 1-56 and 71-80 and complementary sequences thereof.

In certain aspects the invention provides isolated nucleic acids that include at least 17 contiguous nucleotides of the sequences set out in SEQ ID NO: 1-56 and 71-80 and complementary sequences thereof.

In certain aspects the invention provides nucleic acids from Gb, including the nucleic acids set out above, that are about 80% or less identical to the honey bee ortholog of said nucleic acid.

In certain aspects the invention provides vectors that include nucleic acids from Gb, or reverse compliments of such sequences, operably linked to an expression control sequence.

In certain aspects the invention provides host cells transformed with and/or harboring vectors that include nucleic acids from Gb, or reverse compliments of such sequences, operably linked to an expression control sequence.

In certain aspects the invention provides plant tissues, for example, leaf tissue and seeds, transformed with and/or harboring vectors that include nucleic acids from Gb operably linked to an expression control sequence.

In certain aspects the invention provides isolated small inhibitory ribonucleic acid (siRNA) molecules that inhibit expression of Gb nucleic acids.

In certain aspects the invention provides isolated double stranded ribonucleic acid (dsRNA) molecules that include a first strand of nucleotides that is substantially identical to at least 17 contiguous nucleotides of SEQ ID NO: 1-56 and 71-80 and a second strand of nucleotides that is substantially complementary to the first strand of nucleotides.

In certain aspects the invention provides double stranded ribonucleic acid (dsRNA) molecules with a high level of homology (greater than 80%) to mRNA from Gb (Gb targeting dsRNAs), including the dsRNA molecules set out above, that are about 80% or less identical to the honey bee ortholog of the dsRNA.

In certain aspects the invention provides vectors that include an expression control sequence operatively linked to a nucleotide sequence that is a template for one or both strands of a dsRNA from Gb.

In certain aspects the invention provides host cells transformed with and/or harboring vectors that include an expression control sequence operatively linked to a nucleotide sequence that is a template for one or both strands of a dsRNA from Gb.

In certain aspects the invention provides plant tissue transformed with and/or harboring vectors that include an expression control sequence operatively linked to a nucleotide sequence that is a template for one or both strands of a dsRNA from Gb.

In certain aspects the invention provides isolated small inhibitory ribonucleic acid (siRNA) molecules that inhibit expression of an essential gene of Gb.

In certain aspects the invention provides methods of producing a pest resistant plant by expressing a Gb dsRNA in the plant or in propagative or reproductive material of the plant.

In certain aspects the invention provides methods of producing pest resistant eucalyptus by expressing a Gb RNA in the eucalyptus or in propagative or reproductive material of the eucalyptus.

In certain aspects the invention provides methods of producing eucalyptus resistant to Gb infection and/or infestation by expressing a Gb targeting dsRNA in the eucalyptus or in propagative or reproductive material of the eucalyptus.

In certain aspects the invention provides methods of producing a plant resistant to a plant pathogenic pest by transforming a plant cell with a recombinant DNA construct or combination of constructs that express a dsRNA; regenerating a plant from the transformed plant cell; and growing the transformed plant cell under conditions suitable for the expression of the recombinant DNA construct.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 schematically depicts certain, non-limiting nucleic acids according to the invention. (A) Schematic of silencing construct constructed using sequences from three Gb genes. Transgene P1 (Promoter 1) to T1 (Termination sequence 1) encodes a hairpin RNA (hpRNA) for silencing Gb, constructed by fusing 100 bp from each of three different Gb genes (Gb1, Gb2 and Gb3), by synthesizing the resulting sequence as an inverted repeat, and inserting a loop sequence between the respective sense and inverted repeat sequences. Transgene P2 (Promoter 2) to T2 (termination sequence 2) encodes an mRNA with the respective fused 100 bp sequences from the three Gb genes. mRNA transcribed from transgene P2 to T2 is the template for cytoplasmic enhancement of the silencing signal. (B) Schematic of hpRNA molecule produced by transcription of transgene P1 to T1. (C) Schematic of mRNA produced by transcription of transgene P2 to T2.

FIG. 2 schematically depicts certain, non-limiting nucleic acids according to the invention. (A) Schematic of silencing construct #1, constructed from sequences from three Gb genes in accordance with the general scheme depicted in FIG. 1 (B) Schematic of hpRNA molecule produced by transcription of transgene P1 to T1. (C) Schematic of mRNA produced by transcription of transgene P2 to T2. Definitions: P1—CaMV 35S Promoter (SEQ ID NO: 57); P2—sgFIMV Promoter (SEQ ID NO: 58); T1—AtActin7 Terminator (SEQ ID NO: 59); T2—Nos Terminator (SEQ ID NO: 60); Gb12—SEQ ID NO: 13; Gb13—SEQ ID NO: 15; Gb29—SEQ ID NO: 27; L—loop sequence site (SEQ ID NO: 61).

FIG. 3 schematically depicts certain, non-limiting nucleic acids according to the invention. (A) Schematic of silencing construct #2, constructed from sequences from three Gb genes in accordance with the general scheme depicted in FIG. 1 (B) Schematic of hpRNA molecule produced by transcription of transgene P1 to T1. (C) Schematic of mRNA produced by transcription of transgene P2 to T2. Definitions: P1—CaMV 35S Promoter (SEQ ID NO: 57); P2—sgFIMV Promoter (SEQ ID NO: 58); T1—AtActin7 Terminator (SEQ ID NO: 59); T2—Nos Terminator (SEQ ID NO: 60); Gb31—SEQ ID NO: 32; Gb35—SEQ ID NO: 38; Gb56—SEQ ID NO: 56; L—loop sequence site (SEQ ID NO: 61).

FIG. 4 schematically depicts certain, non-limiting nucleic acids according to the invention. (A) Schematic of silencing construct #3, constructed from sequences from three Gb genes in accordance with the general scheme depicted in FIG. 1 (B) Schematic of hpRNA molecule produced by transcription of transgene P1 to T1. (C) Schematic of mRNA produced by transcription of transgene P2 to T2. Definitions: P1—CaMV 35S Promoter (SEQ ID NO: 57); P2—sgFIMV Promoter (SEQ ID NO: 58); T1—AtActin7 Terminator (SEQ ID NO: 59); T2—Nos Terminator (SEQ ID NO: 60); Gb41—SEQ ID NO: 44; Gb53—SEQ ID NO: 50; Gb54—SEQ ID NO: 52; L—loop sequence site (SEQ ID NO: 61).

FIG. 5 schematically depicts certain, non-limiting nucleic acids according to the invention. (A) Schematic of silencing construct constructed using sequences from a single Gb gene. Transgene P1 to T1 encodes a hairpin RNA (hpRNA) for silencing Gb, constructed from 100 bp of a Gb gene, by synthesizing the sequence as an inverted repeat, and inserting a loop sequence between the respective sense and inverted repeat sequences. Transgene P2 to T2 encodes an mRNA with the 100 bp sequence from the Gb gene. mRNA transcribed from transgene P2 to T2 is the template for cytoplasmic enhancement of the silencing signal. (B) Schematic of hpRNA molecule produced by transcription of transgene P1 to T1. (C) Schematic of mRNA produced by transcription of transgene P2 to T2.

FIG. 6 schematically depicts certain, non-limiting nucleic acids according to the invention. (A) Schematic of silencing construct constructed using sequences from two Gb genes. Transgene P1 to T1 encodes a hairpin RNA (hpRNA) for silencing Gb, constructed by fusing 100 bp from each of two different Gb genes, by, synthesizing the resulting sequence as an inverted repeat, and inserting a loop sequence between the respective sense and inverted repeat sequences. Transgene P2 to T2 encodes an mRNA with the respective fused 100 bp sequences from the two Gb genes. mRNA transcribed from transgene P2 to T2 is the template for cytoplasmic enhancement of the silencing signal. (B) Schematic of hpRNA molecule produced by transcription of transgene P1 to T1. (C) Schematic of mRNA produced by transcription of transgene P2 to T2.

Like reference symbols in the various drawings indicate like elements.

### DETAILED DESCRIPTION

The inventors have conducted transcriptome sequencing of the natural eucalyptus pest, Gb Thaumastocoris peregrinus (Tp) and mined the respective transcriptomes to identify open reading frames Gb genes that correspond to Gb mRNAs. The identification of Gb RNAs allows for the design of siRNA and dsRNA that mediate downregulation (silencing) of Gb genes. Such siRNA and dsRNAs are thus useful as biological control agents to kill or inhibit the development of Gb and inhibit Gb infection of plants.

Accordingly, the present invention describes a nucleic acid based approach for the control of Gb pests. Such nucleic acid based approaches include, without limitation, approaches based on expression of Gb double-stranded (dsRNA), antisense RNA, and mRNA.

The methods of the invention find practical application in any area of technology where it is desirable to inhibit viability, growth, development or reproduction of Gbs, or to decrease pathogenicity or infectivity of the insect. The methods of the invention further find practical application where it is desirable to specifically down-regulate expression of one or more target genes in a Gb insect. Particularly useful practical applications include, but are not limited to, protecting plants against Gb pest infestation.

In certain aspects, an active ingredient for controlling Gb infestation is a double-stranded RNA (dsRNA) or a nucleic acid that can promote or lead to production of a dsRNA, which can be used as an insecticidal formulation. dsRNA can be expressed in a host plant, plant part, plant cell or seed to protect the plant against Gbs. The sequence of the dsRNA corresponds to part or whole of an essential Gb gene and causes downregulation of the insect target gene via RNA interference (RNAi). As a result of the downregulation of mRNA, the dsRNA prevents expression of the target insect protein and causes death, growth arrest or sterility of the insect. In this aspect, siRNA control of insect growth, for preventing insect infestation of a cell or a plant susceptible to insect infection, is effected by contacting insects with a dsRNA produced by annealed complementary strands, one of which has a nucleotide sequence which is complementary to at least part of the nucleotide sequence of an insect target gene. dsRNA is expressed in plant tissue that is ingested by the insect and then taken up by the insect through the gut, and thereby controls growth or prevents infestation. See Huvenne et al., 2010, J Insect Physiol 56: 227-35.

Gb target genes for siRNA-mediated intervention include are preferably non-redundant, vital genes. Vital target genes may be any gene that when inhibited interferes with growth or survival or pathogenicity or infectivity of the insect. Such vital target genes are essential for viability, growth, development or reproduction of the insect, or any gene that is involved with pathogenicity or infectivity of the insect, such that specific inhibition of the target gene leads to a lethal phenotype or decreases or stops insect infestation. Down regulation of such vital target genes, whose activity cannot be complemented by other related genes, results in significant damage to the pest larvae and provides an efficient pest control system for sessile Gb pests. The target gene may be any of the target genes herein described, for instance a target gene that is essential for the viability, growth, development or reproduction of the pest. Examples of target genes include, for example, genes that are involved in protein synthesis and/or metabolism and/or RNA synthesis and metabolism and/or cellular processes. A slight knockdown of these target genes will have an effect on many other genes and processes ultimately leading to a lethal effect on the target pest. Such a down-regulated target gene will result in the death of the insect, or the reproduction or growth of the insect being stopped or delayed. Such target genes are vital for the viability of the insect and are referred to as vital genes.

Potential target genes may be identified based on homologies to genes in other insect species. Published genome-wide RNAi mediated gene interference libraries (15, 16) may be used to identify genes that are lethal to other organisms when RNAi based on these genes is expressed and incorporated into target pest organisms by ingestion or any other means. Thus genes identified as being RNAi-lethal in Drosophila may be used to screen for orthologs in hymenoptera species. Such hymenoptera orthologs may further be used to screen Gb species for potential targets.

Nucleotide sequences of Gb target genes include, for example, the sequences set out in SEQ ID NO: 1-56 and 71-80 the complements of such sequences, the reverse complements of such sequences, and sequences that selectively hybridize to such sequences and complements under high stringency hybridization conditions. Examples of preferred target genes include, without limitation, genes encoding SEQ ID NO: 11, 14, 26, 30, 37, 55, 43, 49 and 51.

Nucleotide sequences useful for dsRNA-mediated downregulation of Gb target genes include, for example, (i) a sequences set out in SEQ ID NO: 1-56 and 71-80 and the complements of such sequences; (ii) sequences which are at least 70%, 75%, 80%, 85%, 90%, 95%, 96%, 97%, 98%, 99% or 99.9% identical to a sequence set out in SEQ ID NO: 1-56 and 71-80 and the complements of such sequences; (iii) sequences comprising at least 17 contiguous nucleotides of SEQ ID NO: 1-56 and 71-80 and the complements of such sequences; and (iv) sequences that selectively hybridize to such sequences and complements under high stringency hybridization conditions.

An “isolated” nucleic acid as used herein is a nucleic that has been identified and separated and/or recovered from a component of its natural environment.

“Controlling pests” as used herein means killing pests, or preventing pests to develop, or to grow or preventing pests to infect or infest. Controlling pests as used herein also encompasses controlling pest progeny (development of eggs). Controlling pests as used herein also encompasses inhibiting viability, growth, development or reproduction of the pest, or to decrease pathogenicity or infectivity of the pest. The compounds and/or compositions described herein, may be used to keep an organism healthy and may be used curatively, preventively or systematically to control pests or to avoid pest growth or development or infection or infestation.

Particular pests envisaged for control by methods described herein are plant pathogenic insect pests. “Controlling insects” as used herein thus encompasses controlling insect progeny (such as development of eggs). Controlling insects as used herein also encompasses inhibiting viability, growth, development or reproduction of the insect, or decreasing pathogenicity or infectivity of the insect. As used herein, controlling insects may refer to inhibiting a biological activity in an insect, resulting in one or more of the following attributes: reduction in feeding by the insect, reduction in viability of the insect, death of the insect, inhibition of differentiation and development of the insect, absence of or reduced capacity for sexual reproduction by the insect.

The compounds and/or compositions described herein, may be used to keep an organism healthy and may be used curatively, preventively or systematically to control an insect or to avoid insect growth or development or infection or infestation. Thus, the invention may allow previously susceptible organisms to develop resistance against infestation by the insect organism.

The term “complementary to at least part of” refers to a nucleotide sequence that is fully complementary to the nucleotide sequence of the target over more than ten nucleotides, for instance over at least 15, 16, 17, 18, 19, 20, 21, 22, 23, 24 or more contiguous nucleotides. Notwithstanding the above, “complementary to at least part” of may also include complementary sequences that are greater than 80% complementary to a nucleotide sequence of a target sequence over a length of more than 20 nucleotides, for instance over at least 20, 21, 22, 23, 24 or more contiguous nucleotides [13, 14].

In certain aspects, the invention provides a method for down-regulating expression of a target gene in an insect, comprising contacting the insect with a dsRNA, wherein the dsRNA comprises annealed complementary strands, one of which has a nucleotide sequence that is complementary to at least part of the nucleotide sequence of the insect target gene to be down-regulated, whereby the dsRNA is taken up into the insect and thereby down-regulates expression of the insect target gene.

The term “insect” encompasses insects of all types and at all stages of development, including egg, larval or nymphal, pupal and adult stages.

As used herein, the term “plant” encompasses any plant material that it is desired to treat to prevent or reduce insect growth and/or insect infestation. This includes, inter alia, whole plants, seedlings, propagation or reproductive material such as seeds, cuttings, grafts, explants, etc., and also plant cell and tissue cultures. The plant material should express, or have the capability to express, the RNA molecule comprising at least one nucleotide sequence that is the RNA complement of or that represents the RNA equivalent of at least part of the nucleotide sequence of the sense strand of at least one target gene of the pest organism, such that the RNA molecule is taken up by a pest upon plant-pest interaction, said RNA molecule being capable of inhibiting the target gene or down-regulating expression of the target gene by RNA interference.

The terms “down-regulation of gene expression” and “inhibition of gene expression” are used interchangeably and refer to a measurable or observable reduction in gene expression or a complete abolition of detectable gene expression, at the level of protein product and/or mRNA product from the target gene. The down-regulation effect of the dsRNA on gene expression may be calculated as being at least 30%, 40%, 50%, 60%, preferably 70%, 80% or even more preferably 90% or 95% when compared with normal gene expression. Depending on the nature of the target gene, down-regulation or inhibition of gene expression in cells of an insect can be confirmed by phenotypic analysis of the cell or the whole insect or by measurement of mRNA or protein expression using molecular techniques such as RNA solution hybridization, PCR, nuclease protection, Northern hybridization, reverse transcription, gene expression monitoring with a microarray, antibody binding, enzyme-linked immunosorbent assay (ELISA), Western blotting, radioimmunoassay (RIA), other immunoassays, or fluorescence-activated cell analysis (FACS).

Down-regulation of an essential gene leads to growth inhibition. Depending on the assay used, the growth inhibition can be quantified as being greater than about 5%, 10%, more preferably about 20%, 25%, 33%, 50%, 60%, 75%, 80%, most preferably about 90%, 95%, or about 99% as compared to a pest organism that has been treated with control dsRNA.

The “target gene” may be essentially any gene that is desirable to be inhibited because it interferes with growth or pathogenicity or infectivity of the insect. For instance, if the method of the invention is to be used to prevent insect growth and/or infestation then it is preferred to select a target gene which is essential for viability, growth, development or reproduction of the insect, or any gene that is involved with pathogenicity or infectivity of the insect, such that specific inhibition of the target gene leads to a lethal phenotype or decreases or stops insect infestation.

According to one non-limiting embodiment, the target gene is such that when its expression is down-regulated or inhibited using the method of the invention, the insect is killed, or the reproduction or growth of the insect is stopped or retarded. This type of target gene is considered to be essential for the viability of the insect and is referred to as essential genes. Therefore, the present invention encompasses a method as described herein, wherein the target gene is an essential gene.

Without being bound by theory, the target gene is such that when it is down-regulated the infestation or infection by the insect, the damage caused by the insect, and/or the ability of the insect to infest or infect host organisms and/or cause such damage, is reduced. The terms “infest” and “infect” or “infestation” and “infection” are generally used interchangeably throughout. This type of target genes is considered to be involved in the pathogenicity or infectivity of the insect. Therefore, the present invention extends to methods as described herein, wherein the target gene is involved in the pathogenicity or infectivity of the insect. The advantage of choosing the latter type of target gene is that the insect is blocked to infect further plants or plant parts and is inhibited to form further generations.

In dsRNA-mediated methods of controlling growth or infestation of a specific insect in or on a host cell or host organism, it is preferred that the dsRNA does not share any significant homology with any host gene, or at least not with any essential gene of the host. In this context, it is preferred that the dsRNA shows less than 30%, more preferably less that 20%, more preferably less than 10%, and even more preferably less than 5% nucleic acid sequence identity with any gene of the host cell. Percent sequence identity should be calculated across the full length of the dsRNA region. If genomic sequence data is available for the host organism one may cross-check sequence identity with the dsRNA using standard bioinformatics tools. In one embodiment, there is no sequence identity between the dsRNA and a host sequences over 21 contiguous nucleotides, meaning that in this context, it is preferred that 21 contiguous base pairs of the dsRNA do not occur in the coding sequences (CDS) of the host organism. In another embodiment, there is less than about 10% or less than about 12.5% sequence identity over 24 contiguous nucleotides of the dsRNA with any nucleotide sequence from a host species.

dsRNA comprises annealed complementary strands, one of which has a nucleotide sequence which corresponds to a target nucleotide sequence of the target gene to be down-regulated. The other strand of the dsRNA is able to base-pair with the first strand.

The expression “target region” or “target nucleotide sequence” of the target insect gene may be any suitable region or nucleotide sequence of the gene. The target region should comprise at least 17, at least 18 or at least 19 consecutive nucleotides of the target gene, more preferably at least 20 or at least 21 nucleotide and still more preferably at least 22, 23 or 24 nucleotides of the target gene.

It is preferred that (at least part of) the dsRNA will share 100% sequence identity with the target region of the insect target gene. However, it will be appreciated that 100% sequence identity over the whole length of the double stranded region is not essential for functional RNA inhibition. RNA sequences with insertions, deletions, and single point mutations relative to the target sequence have also been found to be effective for RNA inhibition.

The terms “corresponding to” or “complementary to” are used herein interchangeably, and when these terms are used to refer to sequence correspondence between the dsRNA and the target region of the target gene, they are to be interpreted accordingly, i.e., as not absolutely requiring 100% sequence identity. However, the percent sequence identity between the dsRNA and the target region will generally be at least 80% or 85% identical, preferably at least 90%, 95%, 96%, or more preferably at least 97%, 98% and still more preferably at least 99%. Two nucleic acid strands are “substantially complementary” when at least 85% of their bases pair.

The term “complementary” as used herein relates to all of DNA-DNA complementarity, RNA-RNA complementarity and to DNA-RNA complementarity. In analogy herewith, the term “RNA equivalent” substantially means that in the DNA sequence(s), the base “T” may be replaced by the corresponding base “U” normally present in ribonucleic acids.

Although dsRNA contains a sequence which corresponds to the target region of the target gene, it is not essential for the whole of the dsRNA to correspond to the sequence of the target region. For example, the dsRNA may contain short non-target regions flanking the target-specific sequence, provided that such sequences do not affect performance of the dsRNA in RNA inhibition to a material extent.

The dsRNA may contain one or more substitute bases in order to optimize performance in RNAi. It will be apparent to one of ordinary skill in the art how to vary each of the bases of the dsRNA in turn and test the activity of the resulting dsRNAs (e.g., in a suitable in vitro test system) in order to optimize the performance of a given dsRNA.

The dsRNA may further contain DNA bases, non-natural bases or non-natural backbone linkages or modifications of the sugar-phosphate backbone, for example to enhance stability during storage or enhance resistance to degradation by nucleases.

Interfering RNAs (siRNAs) of about 21 bp are useful for effective gene silencing. Increasing the length of dsRNA preferably to at least about 80-100 bp may increase the efficiency by which dsRNA is taken up by pest organisms. Such longer fragments may be more effective in gene silencing, possibly due to a more efficient uptake of these long dsRNA by the invertebrate.

RNA duplexes consisting of either 27-mer blunt or short hairpin (sh) RNAs with 29 bp stems and 2-nt 3′ overhangs may also be used as siRNAs. Thus, molecules based upon the targets identified above and being either 27-mer blunt or short hairpin (sh) RNA's with 29-bp stems and 2-nt 3' overhangs are also included within the scope of the invention.

Therefore, in one embodiment, the dsRNA fragment (or region) will itself preferably be at least 17 bp in length, preferably 18 or 19 bp in length, more preferably at least 20 bp, more preferably at least 21 bp, or at least 22 bp, or at least 23 bp, or at least 24 bp, 25 bp, 26 bp or at least 27 bp in length. The expressions “double-stranded RNA fragment” or “double-stranded RNA region” refer to a small entity of the dsRNA corresponding with (part of) the target gene.

More generally, the double stranded RNA is preferably between about 17-1500 bp, even more preferably between about 80-1000 bp and most preferably between about 17-27 bp or between about 80-250 bp; such as double stranded RNA regions of about 17 bp, 18 bp, 19 bp, 20 bp, 21 bp, 22 bp, 23 bp, 24 bp, 25 bp, 27 bp, 50 bp, 80 bp, 100 bp, 150 bp, 200 bp, 250 bp, 300 bp, 350 bp, 400 bp, 450 bp, 500 bp, 550 bp, 600 bp, 650 bp, 700 bp, 900 bp, 100 bp, 1100 bp, 1200 bp, 1300 bp, 1400 bp or 1500 bp.

The upper limit on the length of the dsRNA may be dependent on i) the requirement for the dsRNA to be taken up by the insect and ii) the requirement for the dsRNA to be processed within the cell into fragments that direct RNAi. The chosen length may also be influenced by the method of synthesis of the RNA and the mode of delivery of the RNA to the cell. Preferably the dsRNA to be used in the methods of the invention will be less than 10,000 bp in length, more preferably 1000 bp or less, more preferably 500 bp or less, more preferably 300 bp or less, more preferably 100 bp or less. For any given target gene and insect, the optimum length of the dsRNA for effective inhibition may be determined by experiment.

The dsRNA may be fully or partially double-stranded. Partially dsRNAs may include short single-stranded overhangs at one or both ends of the double-stranded portion, provided that the RNA is still capable of being taken up by insects and directing RNAi. The dsRNA may also contain internal non-complementary regions.

The methods of the invention encompass the simultaneous or sequential provision of two or more different dsRNAs or RNA constructs to the same insect, so as to achieve down-regulation or inhibition of multiple target genes or to achieve a more potent inhibition of a single target gene.

Alternatively, multiple targets are hit by the provision of one dsRNA that hits multiple target sequences, and a single target is more efficiently inhibited by the presence of more than one copy of the double stranded RNA fragment corresponding to the target gene. Thus, in certain aspects, a dsRNA construct comprises multiple dsRNA regions, at least one strand of each dsRNA region comprising a nucleotide sequence that is complementary to at least part of a target nucleotide sequence of an insect target gene. The dsRNA regions in the RNA construct may be complementary to the same or to different target genes and/or the dsRNA regions may be complementary to targets from the same or from different insect species.

The terms “hit”, “hits” and “hitting” are alternative wordings to indicate that at least one of the strands of the dsRNA is complementary to, and as such may bind to, the target gene or nucleotide sequence.

In one embodiment, the double stranded RNA region comprises multiple copies of the nucleotide sequence that is complementary to the target gene. Alternatively, the dsRNA hits more than one target sequence of the same target gene. The invention thus encompasses isolated double stranded RNA constructs comprising at least two copies of said nucleotide sequence complementary to at least part of a nucleotide sequence of an insect target.

The term “multiple” as used herein means at least two, at least three, at least four, at least five, at least six, etc.

The expressions “a further target gene” or “at least one other target gene” mean for instance a second, a third or a fourth, etc. target gene.

dsRNA that hits more than one of the above-mentioned targets, or a combination of different dsRNA against different of the above mentioned targets are developed and used in the methods of the present invention.

dsRNA regions (or fragments) in the double stranded RNA may be combined as follows: a) when multiple dsRNA regions targeting a single target gene are combined, they may be combined in the original order (i.e., the order in which the regions appear in the target gene) in the RNA construct; b) alternatively, the original order of the fragments may be ignored so that they are scrambled and combined randomly or deliberately in any order into the double stranded RNA construct; c) alternatively, one single fragment may be repeated several times, for example from 1 to 10 times, e.g., 1, 2, 3, 4, 5, 6, 7, 8, 9 or 10 times, in the ds RNA construct, or d) the dsRNA regions (targeting a single or different target genes) may be combined in the sense or antisense orientation.

Multiple dsRNA regions targeting a single or different weak gene(s) may be combined to obtain a stronger RNAi effect. “Insect specific” genes or sequences, e.g., Gb specific, particularly Gb specific genes and sequences, encompass genes that have no substantial homologous counterpart in non-insect organisms as can be determined by bioinformatics homology searches, for example by BLAST searches. The choice of a specific target gene results in a species specific RNAi effect, with no effect or no substantial (adverse) effect in non-target organisms. “Conserved genes” encompass genes that are conserved (at the amino acid level) between the target organism and non-target organism(s). To reduce possible effects on non-target species, such effective but conserved genes are analyzed and target sequences from the variable regions of these conserved genes are chosen to be targeted by the dsRNA regions in the RNA construct. Conservation is assessed at the level of the nucleic acid sequence. Such variable regions thus encompass the least conserved sections, at the level of the nucleic acid sequence, of the conserved target gene(s). The RNA constructs according to the present invention target multiple genes from different biological pathways, resulting in a broad cellular RNAi effect and more efficient insect control. In certain embodiments dsRNAs are constructed from sequences, e.g., Gb transcriptome sequences, that are equal to or less than 80% identical to the sequence of a honey bee ortholog.

In certain aspects, dsRNA constructs are constructed with gene sequences that affect different classes of cellular functions. Examples of such classes of cellular function include, without limitation, (i) protein synthesis and metabolism, (ii) RNA synthesis and metabolism, and (iii) cellular processes. In certain embodiments, dsRNA constructs comprise sequences from each of the aforementioned claims, i.e., three classes. In certain embodiments, dsRNA constructs comprise sequences from two of the aforementioned classes, e.g., protein synthesis and metabolism and RNA synthesis and metabolism; protein synthesis and cellular processes; or RNA synthesis and metabolism and cellular processes.

dsRNA regions comprise at least one strand that is complementary to at least part or a portion of the nucleotide sequence of any of the target genes herein described. However, provided one of the double stranded RNA regions comprises at least one strand that is complementary to a portion of the nucleotide sequence of any one of the target genes herein described, the other double stranded RNA regions may comprise at least one strand that is complementary to a portion of any other insect target gene (including known target genes).

In some constructs, dsRNAs may comprise additional sequences and optionally a linker. Additional sequences may include, for example, (i) a sequence facilitating large-scale production of the dsRNA construct; (ii) a sequence effecting an increase or decrease in the stability of the dsRNA; (iii) a sequence allowing the binding of proteins or other molecules to facilitate uptake of the RNA construct by insects; (iv) a sequence which is an aptamer that binds to a receptor or to a molecule on the surface or in the cytoplasm of an insect to facilitate uptake, endocytosis and/or transcytosis by the insect; or (v) additional sequences to catalyze processing of dsRNA regions. In one embodiment, the linker is a conditionally self-cleaving RNA sequence, preferably a pH sensitive linker or a hydrophobic sensitive linker.

Multiple dsRNA regions of the dsRNA construct may be connected directly or by one or more linkers. A linker may be present at a site in the RNA construct, separating dsRNA regions from another region of interest. Multiple dsRNA regions of dsRNA constructs may be connected without linkers.

When present, linkers may be used to disconnect smaller dsRNA regions in the pest organism. Advantageously, in this situation the linker sequence may promote division of a long dsRNA into smaller dsRNA regions under particular circumstances, resulting in the release of separate dsRNA regions under these circumstances and leading to more efficient gene silencing by these smaller dsRNA regions. Examples of suitable conditionally self-cleaving linkers are RNA sequences that are self-cleaving at high pH conditions. Suitable examples of such RNA sequences are described by Borda et al. (Nucleic Acids Res. 2003 May 15; 31(10):2595-600), which document is incorporated herein by reference. This sequence originates from the catalytic core of the hammerhead ribozyme HH16.

Linkers may also be located at a site in the dsRNA construct, separating the dsRNA regions from another, e.g., an additional, sequence of interest, which preferably provides some additional function to the RNA construct.

dsRNA constructs may include aptamers to facilitate uptake of the dsRNA by the insect. The aptamer is designed to bind a substance which is taken up by the insect. Such substances may be from an insect or plant origin. One specific example of an aptamer, is an aptamer that binds to a transmembrane protein, for example a transmembrane protein of an insect. Alternatively, the aptamer may bind a (plant) metabolite or nutrient which is taken up by the insect.

Linkers may undergo self-cleaving in the endosome. This may be advantageous when the constructs of the present invention are taken up by the insect via end ocytosis or transcytosis, and are therefore compartmentalized in the endosomes of the insect species. The endosomes may have a low pH environment, leading to cleavage of the linker.

Linkers that are self-cleaving in hydrophobic conditions are particularly useful in dsRNA constructs when used to be transferred from one cell to another via the transit in a cell wall, for example when crossing the cell wall of an insect pest organism.

An intron may be used as a linker. An “intron” as used herein may be any non-coding RNA sequence of a messenger RNA.

A non-complementary RNA sequence, ranging from about 1 base pair to about 10,000 base pairs, may also be used as a linker.

Without wishing to be bound by any particular theory or mechanism, it is thought that long dsRNAs are taken up by the insect from their immediate environment. dsRNAs taken up into the gut and transferred to the gut epithelial cells are then processed within the cell into short dsRNAs, called small interfering RNAs (siRNAs), by the action of an endogenous endonuclease. The resulting siRNAs then mediate RNAi via formation of a multi-component RNase complex termed the RISC or RNA interfering silencing complex.

In order to achieve down-regulation of a target gene within an insect cell the dsRNA added to the exterior of the cell wall may be any dsRNA or dsRNA construct that can be taken up into the cell and then processed within the cell into siRNAs, which then mediate RNAi, or the RNA added to the exterior of the cell could itself be an siRNA that can be taken up into the cell and thereby direct RNAi.

siRNAs are generally short dsRNAs having a length in the range of from 19 to 25 base pairs, or from 20 to 24 base pairs. In preferred embodiments siRNAs having 19, 20, 21, 22, 23, 24 or 25 base pairs, and in particular 21 or 22 base pairs, corresponding to the target gene to be down-regulated may be used. However, the invention is not intended to be limited to the use of such siRNAs.

siRNAs may include single-stranded overhangs at one or both ends, flanking the double-stranded portion. The siRNA may contain 3′ overhanging nucleotides, preferably two 3′ overhanging thymidines (dTdT) or uridines (UU). 3′ TT or UU overhangs may be included in the siRNA if the sequence of the target gene immediately upstream of the sequence included in double-stranded part of the dsRNA is AA. This allows the TT or UU overhang in the siRNA to hybridize to the target gene. Although a 3′ TT or UU overhang may also be included at the other end of the siRNA it is not essential for the target sequence downstream of the sequence included in double-stranded part of the siRNA to have AA. In this context, siRNAs which are RNA/DNA chimeras are also contemplated. These chimeras include, for example, the siRNAs comprising a dsRNA with 3′ overhangs of DNA bases (e.g., dTdT), as discussed above, and also dsRNAs which are polynucleotides in which one or more of the RNA bases or ribonucleotides, or even all of the ribonucleotides on an entire strand, are replaced with DNA bases or deoxyribonucleotides.

dsRNA may be formed from two separate (sense and antisense) RNA strands that are annealed together by (non-covalent) base pairing. Alternatively, the dsRNA may have a foldback stem-loop or hairpin structure, wherein the two annealed strands of the dsRNA are covalently linked. In this embodiment the sense and antisense stands of the dsRNA are formed from different regions of single polynucleotide molecule that is partially self-complementary. RNAs having this structure are convenient if the dsRNA is to be synthesized by expression in vivo, for example in a host cell or organism, or by in vitro transcription. The precise nature and sequence of the “loop” linking the two RNA strands is generally not material to the invention, except that it should not impair the ability of the double-stranded part of the molecule to mediate RNAi. The features of “hairpin” or “stem-loop” RNAs for use in RNAi are generally known in the art (see for example WO 99/53050, the contents of which are incorporated herein by reference). In other embodiments of the invention, the loop structure may comprise linker sequences or additional sequences as described above.

In certain aspects, the Gb sequences disclosed herein and the complements of such sequences may also be used to inhibit expression of Gb nucleic acids via expression of antisense RNA or overexpression of sense RNA, using methods well known in the art. See, e.g., Frizzi et al., Plant Biotech J, (2010) 8:655-677; Brodersen et al., Trends in Genetics, (2008) 22:268-280; and U.S. Pat. No. 5,759,829. Using expression elements, vectors and methods described herein, antisense RNAs or sense RNAs for Gb target genes are expressed in eucalyptus plants. Upon ingestion by Gb pests, the antisense or sense RNAs inhibit expression of the target genes to control pest infestation.

Target nucleotide sequences for design the dsRNA constructs are preferably at least 17, preferably at least 18, 19, 20 or 21, more preferably at least 22, 23 or 24 nucleotides in length. Non-limiting examples of preferred target nucleotide sequences are given in the examples.

Target sequences may include sequences that are homologous to sequences disclosed herein. Homologues of target genes can be found using methods well known to those of ordinary skill in the art. Preferred homologues are genes comprising a sequence which is at least about 85% or 87.5%, still more preferably about 90%, still more preferably at least about 95% and most preferably at least about 99% or 99.9% identical to a sequence disclosed herein, or the complement thereof. Methods for determining sequence identity are routine in the art and include use of the Blast software and EMBOSS software (The European Molecular Biology Open Software Suite (2000), Rice, P. Longden, I. and Bleasby, A. Trends in Genetics 16, (6) pp 276-277). The term “identity” as used herein refers to the relationship between sequences at the nucleotide level. The expression “% identical” is determined by comparing optimally aligned sequences, e.g., two or more, over a comparison window wherein the portion of the sequence in the comparison window may comprise insertions or deletions as compared to the reference sequence for optimal alignment of the sequences. The reference sequence does not comprise insertions or deletions. The reference window is chosen from between at least 10 contiguous nucleotides to about 50, about 100 or to about 150 nucleotides, preferably between about 50 and 150 nucleotides. “percent identity” is then calculated by determining the number of nucleotides that are identical between the sequences in the window, dividing the number of identical nucleotides by the number of nucleotides in the window and multiplying by 100.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)-0.61 form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used.

An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, N.Y. (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C. and a wash in 0.1×SSC, 0.1% SDS at 65° C.

dsRNA may be expressed by (e.g., transcribed within) a host cell or host organism. The host cell or organism may or may not be a host cell or organism susceptible or vulnerable to infestation by an insect. If the host cell or organism is a host cell or organism susceptible or vulnerable to infestation by an insect, RNAi-mediated gene silencing of one or more target genes in the insect may be used as a mechanism to control growth of the insect in or on the host organism and/or to prevent or reduce insect infestation of the host organism. Expression of the dsRNA within cells of the host organism may thus confer resistance to a particular insect or to a class of insects. In case the dsRNA hits more than one insect target gene, expression of the dsRNA within cells of the host organism may confer resistance to more than one insect or more than one class of insects.

In a preferred embodiment the host organism is a plant and the insect is a plant pathogenic insect. In this embodiment the insect is contacted with the dsRNA by expressing the dsRNA in a plant, plant tissue or plant cell that is infested with or susceptible to infestation with, or ingestion by, the plant pathogenic insect. A preferred plant host organism is eucalyptus. Examples of eucalyptus include, without limitation, the following species: E. botryoides, E. bridgesiana, E. camaldulensis, E. cinerea, E. globule, E. grandis, E. gunii, E. nicholii, E. pulverulenta, E. robusta, E. rudis, E. saligna, E. Tereticornis, E. Urophilla, E. viminalis and a cross hybrids of any of the preceding species especially Eucalyptus grandis and Eucalyptus urophylla. A preferred plant pathogenic insect is a Gb, e.g., Gb.

The term “plant” encompasses any plant material that it is desired to treat to prevent or reduce insect growth and/or insect infestation. This includes, inter alia, whole plants, seedlings, propagation or reproductive material such as seeds, cuttings, grafts, explants, etc. and also plant cell and tissue cultures. The plant material should express, or have the capability to express, dsRNA corresponding to one or more target genes of the insect.

In certain aspects the invention provides a plant, preferably a transgenic plant, or propagation or reproductive material for a (transgenic) plant, or a plant cell culture expressing or capable of expressing at least one dsRNA, wherein the dsRNA comprises annealed complementary strands, one of which has a nucleotide sequence which is complementary to at least part of a target nucleotide sequence of a target gene of an insect, such that the dsRNA is taken up by an insect upon plant-insect interaction, said double stranded RNA being capable of inhibiting the target gene or down-regulating expression of the target gene by RNA interference. The target gene may be any of the target genes herein described, for instance a target gene that is essential for the viability, growth, development or reproduction of the insect.

A plant may be provided in a form that is actively expressing (transcribing) a dsRNA in one or more cells, cell types or tissues. Alternatively, a plant may be “capable of expressing”, meaning that it is transformed with a transgene which encodes the desired dsRNA but that the transgene is not active in the plant when (and in the form in which) the plant is supplied. A recombinant DNA construct comprising a nucleotide sequence encoding a dsRNA or dsRNA construct may be thus be operably linked to at least one regulatory sequence. Preferably, the regulatory sequence is selected from the group comprising constitutive promoters or tissue specific promoters as described below.

A target gene may be any target gene herein described. Preferably a regulatory element is a regulatory element that is active in a plant cell. More preferably, the regulatory element is originating from a plant. The term “regulatory sequence” is to be taken in a broad context and refers to a regulatory nucleic acid capable of effecting expression of the sequences to which it is operably linked.

Encompassed by the aforementioned term are promoters and nucleic acids or synthetic fusion molecules or derivatives thereof which activate or enhance transcription of a nucleic acid, so called activators or enhancers. The term “operably linked” as used herein refers to a functional linkage between the promoter sequence and the gene of interest, such that the promoter sequence is able to initiate transcription of the gene of interest.

By way of example, the transgene nucleotide sequence encoding the dsRNA could be placed under the control of an inducible or growth or developmental stage-specific promoter which permits transcription of the dsRNA to be turned on, by the addition of the inducer for an inducible promoter or when the particular stage of growth or development is reached.

Alternatively, the transgene encoding the dsRNA is placed under the control of a strong constitutive promoter such as any selected from the group comprising the CaMV35S promoter, doubled CaMV35S promoter, ubiquitin promoter, actin promoter, rubisco promoter, GOS2 promoter, Figwort mosaic virus (FMV) 34S promoter, cassaya vein mosaic virus (CsVMV) promoter (Verdaguer B. et al, Plant Mol. Biol. 1998 37(6):1055-67).

Alternatively, the transgene encoding the dsRNA is placed under the control of a tissue specific promoter such as any selected from the group comprising root specific promoters of genes encoding PsMTA Class III chitinase, photosynthetic tissue-specific promoters such as promoters of cab1 and cab2, rbcS, gapA, gapB and ST-LS1 proteins, JAS promoters, chalcone synthase promoter and promoter of RJ39 from strawberry.

A transgene encoding the dsRNA may also be placed under the control of an insect-induced promoter, for instance the potato proteinase inhibitor II (PinII) promoter (Duan X et al, Nat. Biotechnol. 1996, 14(4):494-8)); or a wounding-induced promoter, for instance the jasmonates and ethylene induced promoters, PDF1.2 promoter (Manners J M et al., Plant Mol. Biol. 1998, 38(6):1071-80); or under a defense related promoter, for instance the salicylic acid induced promoters and plant-pathogenesis related protein (PR protein) promoters (PR1 promoter (Cornelissen B J et al., Nucleic Acids Res. 1987, 15(17):6799-811; COMT promoter (Toquin V et al, Plant Mol. Biol. 2003, 52(3):495-509).

When using the methods described herein for developing transgenic plants resistant against insects, it may be beneficial to place the nucleic acid encoding the dsRNA under the control of a tissue-specific promoter. In order to improve the transfer of the dsRNA from the plant cell to the pest, the plants could preferably express the dsRNA in a plant part that is first accessed or damaged by the plant pest. In case of plant pathogenic insects, preferred tissues to express the dsRNA are the leaves, stems, roots, and seeds. Therefore, in the methods disclosed herein, a plant tissue-preferred promoter may be used, such as a leaf-specific promoter, a stem-specific promoter, a phloem-specific promoter, a xylem-specific promoter, a root-specific promoter, or a seed-specific promoter (sucrose transporter gene AtSUC promoter (Baud S et al., Plant J. 2005, 43(6):824-36), wheat high molecular weight glutenin gene promoter (Robert L S et al., Plant Cell. 1989, 1(6):569-78.)).

Suitable examples of a root specific promoter are PsMTA (Fordam-Skelton, A. P., et al., 1997 Plant Molecular Biology 34: 659-668.) and the Class III Chitinase promoter. Examples of leaf- and stem-specific or photosynthetic tissue-specific promoters that are also photoactivated are promoters of two chlorophyll binding proteins (cab1 and cab2) from sugar beet (Stahl D. J., et al., 2004 BMC Biotechnology 2004 4:31), ribulose-bisphosphate carboxylase (Rubisco), encoded by rbcS (Nomura M. et al., 2000 Plant Mol. Biol. 44: 99-106), A (gapA) and B (gapB) subunits of chloroplast glyceraldehyde-3-phosphate dehydrogenase (Conley T. R. et al. 1994 Mol. Cell. Biol. 19: 2525-33; Kwon H. B. et al. 1994 Plant Physiol. 105: 357-67), promoter of the Solanum tuberosum gene encoding the leaf and stem specific (ST-LS1) protein (Zaidi M. A. et al., 2005 Transgenic Res. 14:289-98), stem-regulated, defense-inducible genes, such as JAS promoters (patent publication no. 20050034192/US-A1). An example of a flower-specific promoter is for instance, the chalcone synthase promoter (Faktor 0. et al. 1996 Plant Mol. Biol. 32: 849) and an example of a fruit-specific promoter is for instance RJ39 from strawberry (WO 98 31812).

Other promoters useful for the expression of dsRNA are used and include, but are not limited to, promoters from an RNA Poll, an RNA Poll, an RNA PolIII, T7 RNA polymerase or SP6 RNA polymerase. These promoters are typically used for in vitro-production of dsRNA, which dsRNA is then included in an anti-insecticidal agent, for example, in an anti-insecticidal liquid, spray or powder.

The dsRNA or RNA constructs described herein may be generated by the steps of (i) contacting an isolated nucleic acid or a recombinant DNA construct with cell-free components; or (ii) introducing (e.g., by transformation, transfection or injection) an isolated nucleic acid or a recombinant DNA construct into a cell, under conditions that allow transcription of the nucleic acid or recombinant DNA construct to produce the dsRNA or RNA construct.

Optionally, one or more transcription termination sequences may also be incorporated in the recombinant construct. The term “transcription termination sequence” encompasses a control sequence at the end of a transcriptional unit, which signals 3′ processing and poly-adenylation of a primary transcript and termination of transcription. Additional regulatory elements, such as transcriptional or translational enhancers, may be incorporated in the expression construct.

Recombinant constructs may further include an origin of replication which is required for maintenance and/or replication in a specific cell type. One example is when an expression construct is required to be maintained in a bacterial cell as an episomal genetic element (e.g., plasmid or cosmid molecule) in a cell. Preferred origins of replication include, but are not limited to, fl-ori and colE1 ori.

Recombinant construct may optionally include a selectable marker gene. As used herein, the term “selectable marker gene” includes any gene, which confers a phenotype on a cell in which it is expressed to facilitate the identification and/or selection of cells, which are transfected or transformed, with an expression construct of the invention. Examples of suitable selectable markers include resistance genes against ampicillin (Amp^{r}), tetracycline (Tc^{r}), kanamycin (Kan^{r}), phosphinothricin, and chloramphenicol (CAT) gene. Other suitable marker genes provide a metabolic trait, for example manA. Visual marker genes may also be used and include for example beta-glucuronidase (GUS), luciferase and Green Fluorescent Protein (GFP).

Plants that have been stably transformed with a transgene encoding the dsRNA may be supplied as seed, reproductive material, propagation material or cell culture material which does not actively express the dsRNA but has the capability to do so. The plant may be provided in a form wherein it is actively expressing (transcribing) the RNA molecule in one or more cells, cell types or tissues. Alternatively, the plant may be “capable of expressing”, meaning that it is transformed with a transgene which encodes the desired RNA molecule but that the transgene is not active in the plant when (and in the form in which) the plant is supplied. Many vectors are available for this purpose, and selection of the appropriate vector will depend mainly on the size of the nucleic acid to be inserted into the vector and the particular host cell to be transformed with the vector.

General techniques for expression of exogenous dsRNA in plants for the purposes of RNAi are known in the art (see Baulcombe D, 2004, Nature. 431(7006):356-63. RNA silencing in plants, the contents of which are incorporated herein by reference). More particularly, methods for expression of dsRNA in plants for the purposes of down-regulating gene expression in plant pests such as nematodes or insects are also known in the art. Similar methods can be applied in an analogous manner in order to express dsRNA in plants for the purposes of down-regulating expression of a target gene in a plant pathogenic insect. In order to achieve this effect it is necessary only for the plant to express (transcribe) the dsRNA in a part of the plant which will come into direct contact with the insect, such that the dsRNA can be taken up by the insect. Depending on the nature of the insect and its relationship with the host plant, expression of the dsRNA could occur within a cell or tissue including the vasculature of a plant within which the insect is also present during its life cycle, or the RNA may be secreted into a space between cells, such as the apoplast, that is occupied by the insect during its life cycle.

Furthermore, the dsRNA may be located in the plant cell, for example in the cytosol, or in the plant cell organelles such as a chloroplast, mitochondrion, vacuole or endoplastic reticulum. dsRNA may further be expressed in and/or transported to the phloem, e.g., leaf phloem, or xylem, where it may be taken up by sap sucking pests. See Pitino et al., PLoS ONE, 6(10):e25709 (2011) and Mlotshwa et al., Plant Cell, 14:S289-S301 (2002).

During development, Gb larvae are exposed to the extracellular environment including the vasculature and to intracellular contents, due to ingestion (e.g., ingestion of apoplasts) or cell lysis.

Alternatively, the dsRNA may be secreted by the plant cell and by the plant to the exterior of the plant. As such, the dsRNA may form a protective layer on the surface of the plant.

In a further aspect, the invention also provides combinations of methods and compositions for preventing or protecting plants from pest infestation. For instance, one means provides using the plant transgenic approach combining methods using expression of dsRNA molecules and methods using expression of Bt insecticidal proteins.

In a further embodiment, the invention relates to a composition for controlling insect growth and/or preventing or reducing insect infestation, comprising at least a plant part, plant cell, plant tissue or seed comprising at least one dsRNA, wherein said dsRNA comprises annealed complementary strands, one of which has a nucleotide sequence which is complementary to at least part of a nucleotide sequence of an insect target gene. Optionally, the composition further comprises at least one suitable carrier, excipient or diluent. The target gene may be any target gene described herein. Preferably the insect target gene is essential for the viability, growth, development or reproduction of the insect.

Whenever the term “a” is used within the context of “a target gene”, this means “at least one” target gene. The same applies for “a” target organism meaning “at least one” target organism, and “a” RNA molecule or host cell meaning “at least one” RNA molecule or host cell.

According to one embodiment, the methods of the invention rely on uptake by the insect of dsRNA present outside of the insect (e.g., by feeding) and does not require expression of dsRNA within cells of the insect. In addition, the present invention also encompasses methods as described above wherein the insect is contacted with a composition comprising the dsRNA.

The invention further provides a method for down-regulating expression of at least one target gene in a target organism (which is capable of ingesting a plant, plant part, plant cell or seeds) comprising feeding a plant, plant part, plant cell or seed to the target organism which plant, plant part, plant cell or seed expresses dsRNA.

In a more preferred aspect, the invention provides a method for down-regulating expression of at least one target gene in a target organism (which is capable of ingesting a host cell, or extracts thereof) comprising feeding a host plant, plant part, plant cell or seed to the target organism which host plant, plant part, plant cell or seed expresses a dsRNA molecule comprising a nucleotide sequence complementary to or representing the RNA equivalent of at least part of the nucleotide sequence of the at least one target gene, whereby the ingestion of the host cell, host plant, plant part, plant cell or seed by the target organism causes and/or leads to down-regulation of expression of the at least one target gene.

The invention provides for use of a plant, plant part, plant cell or seed as defined herein for down regulation of expression of an insect target gene. In more detailed terms, the invention provides for use of a host cell as defined herein and/or an RNA molecule comprising a nucleotide sequence that is the RNA complement of or that represents the RNA equivalent of at least part of the nucleotide sequence of a target gene from a target organism, as produced by transcription of a nucleic acid molecule in a plant, plant part, plant cell or seed, for instance in the manufacture of a commodity product, for down regulation of expression of a target gene.

According to one embodiment, the methods of the invention rely on a genetically modified organism (GMO) approach wherein the dsRNA is expressed by a cell or an organism infested with or susceptible to infestation by insects. Preferably, said cell is a plant cell or said organism is a plant.

For siRNA mediated downregulation of insect genes, dsRNA is introduced and/or expressed in an insect cell, either directly or indirectly. dsRNA can be added to an insect diet artificially or produced by a transgenic source of food such as bacteria and plants [2,8]. Transgenic plants transcribing inverted repeat RNAs comprised of insect gene specific sequences, can process it to dsRNA and later into siRNA (small interfering RNA that are the first product in the silencing pathway). Insects digesting such transgenic plants are affected by the plant synthesized dsRNA and siRNA [5]. This insect control method can be utilized to protect plants efficiently against specific pests [2,8]. It is not required, however, that dsRNA be processed to siRNA in plant material. dsRNA may be ingested by the insect pest and processed to siRNA for the first time within the insect cell.

Numerous methods for introducing foreign genes into plants are known and can be used to insert an NT polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch et al., Science 227:1229-31 (1985)), electroporation, micro-injection, and biolistic bombardment.

Expression cassettes and vectors and in vitro culture methods for plant cell or tissue transformation and regeneration of plants are known and available. See, e.g., Gruber et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334; and U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722), and ballistic particle acceleration (see, for example, Sanford, et al., U.S. Pat. No. 4,945,050; WO 91/10725; and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. 0. L. Gamborg & G. C. Phillips. Springer-Verlag Berlin Heidelberg N.Y., 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839; and Gordon-Kamm, et al., (1990) Plant Cell 2:603-618 (maize); Hooydaas-Van Slogteren & Hooykaas (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. G. P. Chapman, et al., pp. 197-209. Longman, N.Y. (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255; and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185); all of which are herein incorporated by reference.

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra; and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993; and Simpson, et al., (1986) Plant Mol. Biol. 6:403-15m all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. The selection of either A. tumefaciens or A. rhizogenes will depend on the plant being transformed thereby. In general A. tumefaciens is the preferred organism for transformation. Most dicotyledonous plants, some gymnosperms, and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can now be transformed with some success. European Patent Application No. 604 662 A1 discloses a method for transforming monocots using Agrobacterium. European Application No. 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra; and U.S. patent application Nos. 913,913 and 913,914, both filed Oct. 1, 1986, as referenced in U.S. Pat. No. 5,262,306, issued Nov. 16, 1993, the entire disclosures therein incorporated herein by reference.

Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206; and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731; and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161; and Draper, et al., (1982) Plant Cell Physiol. 23:451.

Electroporation of protoplasts and whole cells and tissues has also been described. See, e.g., Donn, et al., (1990) Abstracts of the VIIth Int'l. Congress on Plant Cell and Tissue Culture IAPTC, A2-38, p. 53; D'Halluin, et al., (1992) Plant Cell 4:1495-505; and Spencer, et al., (1994) Plant Mol. Biol. 24:51-61.

Following stable transformation, plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant.

Transformed plant may be regenerated by micropropagation which provides a rapid, consistent reproduction of the transformed plants. Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

In certain aspects the invention provides methods of producing a plant resistant to a plant pathogenic pest by transforming a plant cell with a recombinant DNA construct or combination of constructs that express a dsRNA; regenerating a plant from the transformed plant cell; and growing the transformed plant cell under conditions suitable for the expression said recombinant DNA construct.

The methods of the invention are applicable to Gb species that are susceptible to gene silencing by RNA interference and that are capable of internalizing dsRNA from their immediate environment. The invention is applicable to the insect at any stage in its development. Because insects have a non-living exoskeleton, they cannot grow at a uniform rate and rather grow in stages by periodically shedding their exoskeleton. This process is referred to as molting or ecdysis. The stages between molts are referred to as “instars” and these stages may be targeted according to the invention. Also, insect eggs or live young may also be targeted according to the present invention. All stages in the developmental cycle, which includes metamorphosis in the pterygotes, may be targeted according to the present invention. Thus, individual stages such as larvae, pupae, nymph etc. stages of development may all be targeted.

Gb are pests for eucalyptus. The nucleic acids, dsRNAs and methods described herein are thus useful for treating or inhibiting Gb infection and infestation of eucalyptus.

### EXAMPLES

### Example 1

### Gb Transcriptome Sequencing

Gb specimens were collected from infected leaves from eucalyptus from Sao Paulo State, Brazil. Total RNA was obtained from a mixture of nymphs at various developmental stages, isolated from lerps, and adults. Batches of 100 specimens were placed in individual microtubes on ice. The tubes were then sealed and immediately frozen in liquid nitrogen and kept at −80° C. until further treatment. Total RNA was isolated using MasterPure RNA purification kit and protocol (MRC85102, Epicentere Biotechnologies). Total RNA volume was 50 μl. Total RNA was then treated with DNAse to remove residual DNA, followed by isolation of poly A mRNA (MicroPoly(A) Purist, Small scale mRNA Purification kit, AM1919 Ambion). mRNA final volume was 20 The purified mRNA was kept at −80° C. until 454 Sequencing was performed. 454 Sequencing was carried out according to standard protocols to provide transcriptomes of the target pest. Sequences were assembled and results annotated on the basis of sequence alignment with known published hemiptera Pea Aphid Acyrthosiphon pisum (Ap) transcriptomes using the Roche software package and annotated using the Blast2Go program, available at http://www.blast2go.org/.

### Example 2

### Identification of Gb Target Genes and Sequences

Unique, vital Gb genes essential either for cellular processes or proper developmental processes of a specific tissue or entire organism were identified as targets for gene silencing. Based on published RNAi libraries in Drosophila melanogaster (Dm) [15, 16] a list was generated of 591 genes that were shown to be lethal in RNAi transgenic Dm. This list was further narrowed to genes that are involved in translation, transcription and development. The resulting subset of 140 genes are involved in one or more of the following: protein synthesis and/or metabolism, RNA synthesis and metabolism and cellular processes.

BLAST (NCBI) comparisons using the 140 genes identified as being lethal when expressed as RNAi in Drosophila were used to identify 128 orthologous sequences from Pea Aphid Acyrthosiphon pisum (Ap). Comparisons using the identified Ap sequences were further used to screen the Gb 454 transcriptome library for potential target genes. Potential Gb target genes were limited to Gb 454 transcriptome sequences that included at least 350 bp in a continuous open reading frame or were at least 50% of the full predicted gene length. The screen of the Gb 454 transcriptome identified 27 potential Gb targets.

The 27 potential Gb targets were further screen to identify sequences that share limited homology to honey bee, Apis mellifera (Ap) sequences. Comparisons were made using a publicly available NCBI Bl2Seq analysis program (available at http://blast.ncbi.nlm.nih.gov/Blast.cgi?PAGE_TYPE=BlastSearch&PROG_DEF=blas tn&BLAST_PROG_DEF=megaBlast&SHOW_DEFAULTS=on&BLAST_SPEC=bla st2seq&LINK_LOC=align2seq) to identify 100 bp sequences from each Gb target that shared limited (i.e., less than 80%) identity to corresponding Am genes (or, when not possible to identify a 100 bp sequence with less than 80% identity to identify, a shorter fragment of such sequences). The regions identified all exhibited 38-74% identity to the respective honey bee sequences.

The respective Gb target genes and the sequences with limited homology to Ap sequences that were identified are set out in SEQ ID NO: 1-58. Table 1 sets out the SEQ ID NOs for the respective Gb target genes and sequences with limited homology identified therein.

TABLE 1
Gb Target Sequences and Fragments With Limited Identity
to honey bee (Apis mellifera) sequences

| | | | Gb Sequence <80% |
|---|---|---|---|
| Gp | | | identical to Am |
| Gene | Dm gene symbol/function | | Sequence |
| No. | (A. mellifera accession no.) | Gb Target Gene | (% identity) |
| 3 | blw/hydrogen-exporting ATPase | SEQ ID NO: 1 | SEQ ID NO: 2 (73) |
| | activity, (XM_392639) | | |
| 7 | Pros28.1A/Proteasome 28 kD | SEQ ID NO: 3 | SEQ ID NO: 4 (70) |
| | subunit 1A; ubiquitin-dependent | | |
| | protein catabolic process | | |
| | (XM_393583) | | |
| 8 | Prosα3T/Proteasome α3T subunit; | SEQ ID NO: 5 | SEQ ID NO: 6 (45) |
| | endopeptidase activity. ubiquitin- | | |
| | dependent protein catabolic process | | |
| | (XM_397196) | | |
| 9 | CG2931/nuclear mRNA splicing, | SEQ ID NO: 7 | SEQ ID NO: 8 (62) |
| | via spliceosome (XM_392161) | | |
| 10 | CG31524/procollagen-proline 4- | SEQ ID NO: 9 | SEQ ID NO: 10 (61) |
| | dioxygenase activity. oxidation- | | |
| | reduction process (XM_392392) | | |
| 12 | CG3590/AMP AMP-lyase; purine | SEQ ID NO: 11 | SEQ ID NO: 12 (52) |
| | nucleotide metabolic process | | |
| | (XM_393961) | | |
| 13 | CG5451/nuclear mRNA splicing, | SEQ ID NO: 14 | SEQ ID NO: 15 (69) |
| | via spliceosome (XM_393446) | | |
| 21 | dlg 1/protein binding. anatomical | SEQ ID NO: 16 | SEQ ID NO: 17 (38) |
| | structure development | | |
| | (XM_393395) | | |
| 24 | e(r)/regulation of transcription | SEQ ID NO: 18 | SEQ ID NO: 19 (74) |
| | from RNA polymerase II promoter | | |
| | (XM_00111990) | | |
| 26 | ebi/regulation of epidermal growth | SEQ ID NO: 20 | SEQ ID NO: 21 (73) |
| | factor receptor signaling pathway; | | |
| | regulation of cell cycle | | |
| | (XM_003251282) | | |
| 27 | EcR/repressing transcription factor | SEQ ID NO: 22 | SEQ ID NO: 23 (61) |
| | binding. anatomical structure | | |
| | development; biological regulation | | |
| | (NM_001159355) | | |
| 28 | Ef1alpha48D/translation | SEQ ID NO: 24 | SEQ ID NO: 25 (71) |
| | elongation factor activity. | | |
| | determination of adult lifespan | | |
| | (NM_001014993) | | |
| 29 | Ef1gamma/translation elongation | SEQ ID NO: 26 | SEQ ID NO: 27 (60) |
| | factor autophagic cell death; | | |
| | salivary gland cell autophagic cell | | |
| | deat (XM_623679) | | |
| 30 | eIF-2alpha/translational initiation | SEQ ID NO: 28 | SEQ ID NO: 29 (42) |
| | (XM_001122232) | | |
| 31 | eIF3-S8/translational initiation | SEQ ID NO: 30 | SEQ ID NO: 31 (66) |
| | (XM_623577) | | |
| 32 | eIF 5/translational initiation | SEQ ID NO: 33 | SEQ ID NO: 34 (54) |
| | (XM_392511) | | |
| 34 | hay/ATP-dependent DNA helicase | SEQ ID NO: 35 | SEQ ID NO: 36 (57) |
| | activity (XM_624122) | | |
| 35 | Hel25E/RNA helicase activity | SEQ ID NO: 37 | SEQ ID NO: 38 (74) |
| | (XM_624891) | | |
| 37 | Hr38/ligand-dependent nuclear | SEQ ID NO: 39 | SEQ ID NO: 40 (58) |
| | receptor activity (NM_001159355) | | |
| 40 | mask/structural constituent of | SEQ ID NO: 41 | SEQ ID NO: 42 (71) |
| | cytoskeleton (XM_393472) | | |
| 41 | mor/transcription coactivator | SEQ ID NO: 43 | SEQ ID NO: 44 (56) |
| | activity (XM_393008) | | |
| 47 | RpS2/structural constituent of | SEQ ID NO: 45 | SEQ ID NO: 46 (65) |
| | ribosome (XM_392843) | | |
| 48 | RpS5a/(XM_624081) structural | SEQ ID NO: 47 | SEQ ID NO: 48 (49) |
| | constituent of ribosome | | |
| 53 | Trip1/translation initiation factor | SEQ ID NO: 49 | SEQ ID NO: 50 (53) |
| | activity (XM_392780) | | |
| 54 | tws/protein serine/threonine | SEQ ID NO: 51 | SEQ ID NO: 52 (67) |
| | phosphatase activity (XM_394082) | | |
| 55 | Ubc-E2H/ubiquitin-protein ligase | SEQ ID NO: 53 | SEQ ID NO: 54 (69) |
| | activity (XM_624081) | | |
| 56 | Uev1A/ubiquitin-conjugating | SEQ ID NO: 55 | SEQ ID NO: 56 (69) |
| | enzyme-like (XM_393411) | | |
| 57 | Vps23/NADH-ubiquinone | SEQ ID NO: 71 | SEQ ID NO: 72 (59) |
| | oxidoreductase, 20 Kd subunit | | |
| | (XM_392437.4) | | |
| 58 | Vps28/Vacuolar protein sorting 28 | SEQ ID NO: 73 | SEQ ID NO: 74 (64) |
| | (XM_392314.4) | | |
| 59 | Vps2/protein transport | SEQ ID NO: 75 | SEQ ID NO: 76 (73) |
| | (XM_625161.3) | | |
| 60 | Vps24/Charged multivesicular body | SEQ ID NO: 77 | SEQ ID NO: 78 (53) |
| | protein 3 (XM_394085.4) | | |
| 61 | Snf7/shrub/ESCRT-III pathway | SEQ ID NO: 79 | SEQ ID NO: 80 (46) |
| | (XM_395324.4) | | |

The identified Gb genes were divided into the following categories:

### Proteins Synthesis and Metabolism:

SEQ ID NO: 3, 5, 18, 22, 24, 26, 28, 30, 33, 45, 47, and 49, respectively.

### Cellular Processes:

SEQ ID NO: 1, 9, 11, 16, 20, 39, 41, 51, 53, and 55 respectively.

### Nucleic Acid Synthesis and Metabolism:

SEQ ID NO: 7, 14, 35, 37, and 43, respectively.

### Example 3

Preparation of dsRNA Triple Gene Silencing Constructs

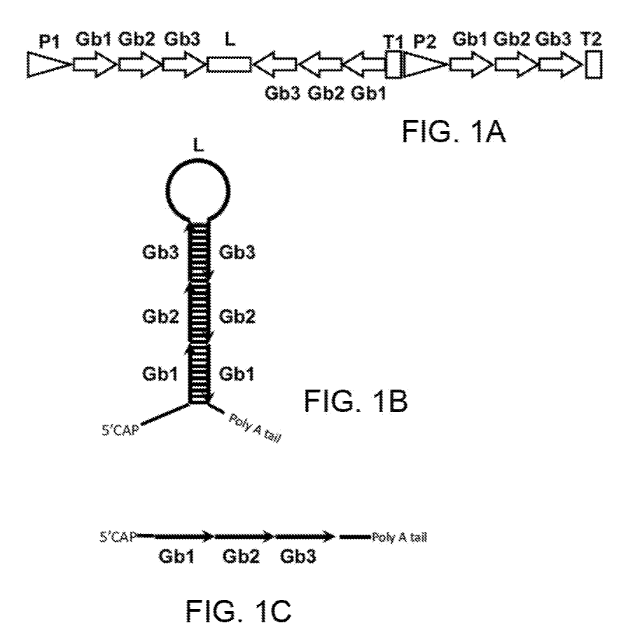

A schematic of the structure of dsRNA triple gene silencing constructs comprising segments from three Gb genes is shown in FIG. 1. Silencing constructs contain two transgenes. A first transgene comprises fragments from each of three Gb genes which are fused and synthesized in inverted repeats, separated by a loop sequence. See FIG. 1A. Transcription of this transgene (initiated at promoter P1 and terminated at T1) produces a hairpin RNA, containing a dsRNA section, formed by annealing of the inverted-repeat sequences of the three Gb genes, and a loop region. See FIG. 1B. A second transgene contains three fused Gb genes, oriented to be transcribed to yield a sense strand with the three gene fragments. See FIGS. 1A and 1C.

The following sequences are used to construct three silencing constructs.

### Silencing Construct #1

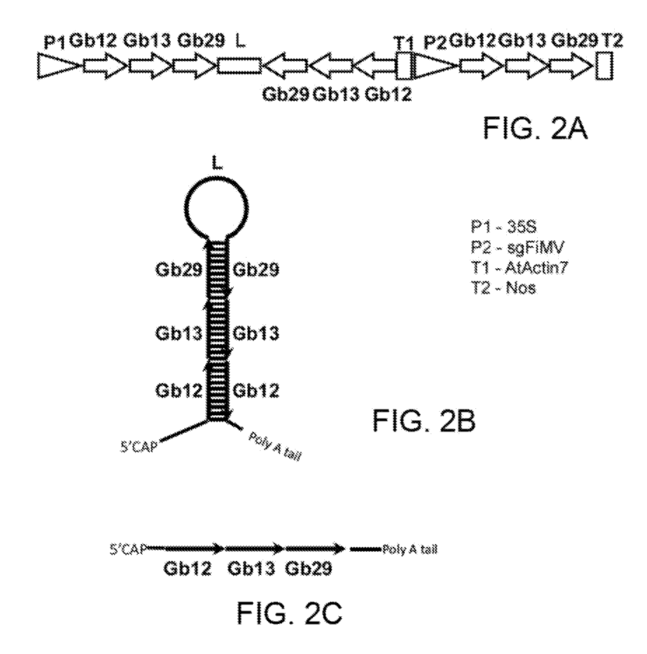

Silencing Construct #1 is shown schematically in FIG. 2. Respective 100 bp fragments of each of the Gb CG 3590 gene (SEQ ID NO: 11) with T→C substitution at position corresponding to nucleotide 136 of SEQ ID NO: 11, to eliminate Xba I site), CG5451gene (SEQ ID NO: 14) and, Tef gene (SEQ ID NO: 26), SEQ ID NO: 13, SEQ ID NO: 15 and SEQ ID NO: 27, respectively, were fused and synthesized in inverted repeats separated by 106 bp of a loop sequence (Loop 1; SEQ ID NO: 61). Transcription initiation was driven by the 35S CaMV promoter (SEQ ID NO: 57). Transcription termination was provided by the AtActin7 Terminator (SEQ ID NO: 59). The select 100 bp of SEQ ID NO: 11, 14 and 26 (respectively, SEQ ID NO: 13, SEQ ID NO: 15 and SEQ ID NO: 27) were synthesized in sense orientation between sgFIMV Promoter (SEQ ID NO: 61) to NOS Terminator (SEQ ID NO: 63).

Transcription of construct 1 would yield two mRNAs: (1) A hairpin RNA (hpRNA) with a stem formed by the reverse complementary sequences of the three Gb 100 bp sequences, to silence the corresponding Gb genes (see FIG. 2B); and (2) sense mRNA of the three, fused Gb genes (see FIG. 2C).

The hpRNA formed upon transcription of the hpRNA-forming transgene of Construct #1 has the following sequence (SEQ ID NO: 62):

AGCTTTAGACCGTCTGGTTACCAAAAAAGCTGGTTTCTCTACTTCTCACA
TCATCTGTGGCCAAACATACCCTAGAAAAGTTGACGTCATCGTAACGGGA
CTCAATTACATCTGGAAAATTGGAGCGTACTCTCAATGTTCACGAGAAAT
TAGTAATTGGATTGACTCATCACCCTCATCAAAATCTCCTAGGAACCTAC
CAACTTTCAGAAAACCATATGTCAATGTTAACAGATGGTTTACTACTATT
GTCAACCAACCAGAATTTAAGAAAATTGTAGGAGAGGTCAAATTATGTGA
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCTCACATAATTTGACCTCTCCTACAATTTTCTTAAATTCTGGTTG
GTTGACAATAGTAGTAAACCATCTGTTAACATTGACATATGGTTTTCTGA
AAGTTGGTAGGTTCCTAGGAGATTTTGATGAGGGTGATGAGTCAATCCAA
TTACTAATTTCTCGTGAACATTGAGAGTACGCTCCAATTTTCCAGATGTA
ATTGAGTCCCGTTACGATGACGTCAACTTTTCTAGGGTATGTTTGGCCAC
AGATGATGTGAGAAGTAGAGAAACCAGCTTTTTTGGTAACCAGACGGTCT
AAAGCT

The respective hpRNA sequences correspond to the following elements: Nucleotides 1-100 and 607-706: Respective sense and reverse complement sequences of SEQ ID NO: 13, corresponding to nucleotides 66-165 of SEQ ID NO: 11, with T136C substitution to eliminate an Xba I site;

Nucleotides 101-200 and 507-606: Respective sense and reverse complement sequences of SEQ ID NO: 15, corresponding to nucleotides 759-858 of SEQ ID NO: 14;

Nucleotides 201-300 and 407-506: Respective sense and reverse complement sequences of SEQ ID NO: 27, corresponding to nucleotides 518-617 of SEQ ID NO: 26; and

Nucleotides 301-406: 106 bp Loop fragment (SEQ ID NO: 61) based on Partial Leptocibe invasa Chitin Synthase intron.

The sense mRNA transcribed from construct 1 has the following sequence (SEQ ID NO: 63):

AGCTTTAGACCGTCTGGTTACCAAAAAAGCTGGTTTCTCTACTTCTCACA
TCATCTGTGGCCAAACATACCCTAGAAAAGTTGACGTCATCGTAACGGGA
CTCAATTACATCTGGAAAATTGGAGCGTACTCTCAATGTTCACGAGAAAT
TAGTAATTGGATTGACTCATCACCCTCATCAAAATCTCCTAGGAACCTAC
CAACTTTCAGAAAACCATATGTCAATGTTAACAGATGGTTTACTACTATT
GTCAACCAACCAGAATTTAAGAAAATTGTAGGAGAGGTCAAATTATGTGA

### Silencing Construct 2

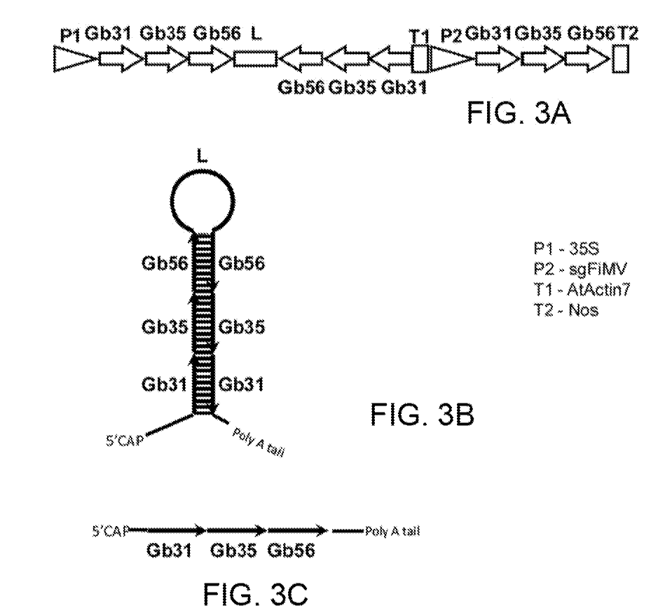

Silencing Construct #2 is shown schematically in FIG. 3. Respective 100 bp fragments of each of the Gb eIF3-58 gene (SEQ ID NO: 30) gene (with T→C substitution at position corresponding to nucleotide 793 of SEQ ID NO: 30, to eliminate Xba I site), Hel25E gene (SEQ ID NO: 37) and, Uev1A gene (SEQ ID NO: 55), SEQ ID NO: 32, SEQ ID NO: 38 and SEQ ID NO: 56, respectively, were fused and synthesized in inverted repeats separated by 106 bp of a loop sequence (Loop 1; SEQ ID NO: 61). Transcription initiation was driven by the 35 S CaMV promoter (SEQ ID NO: 57). Transcription termination was provided by the AtActin7 Terminator (SEQ ID NO: 59). The select 100 bp of SEQ TD NO: 30, 37 and 55 (respectively, SEQ ID NO: 32, SEQ ID NO: 38 and SEQ ID NO: 56) were synthesized in sense orientation between sgFIMV Promoter (SEQ ID NO: 58) to NOS Terminator (SEQ ID NO: 60).

Transcription of construct 2 would yield two mRNAs: (1) A hairpin RNA (hpRNA) with a stem formed by the reverse complementary sequences of the three Gb 100 bp sequences, to silence the corresponding Gb genes (see FIG. 3B); and (2) sense mRNA of the three, fused Gb genes (see FIG. 3C).

The hpRNA formed upon transcription of the hpRNA-forming transgene of Construct #2 has the following sequence (SEQ ID NO: 64):

CCCAAACAGTGGTCCTTCATCGATCTGAACCACCTAGACTTCAAGCGCTA
GCACTTCAATTGGCAGACAAAGTTAATAACTTCGTTGACTCAAATGAACG
GCCTGAAGATTCTGACACTTATCTACACAGAGTGGCACGTGCAGGGCGAT
TCGGCACAAAGGGTTTAGCCATCACCTTTGTTTGTGATGAAAATGATGCT
AGAGTATACTATCAAGTCTTTATTACAAGAACTGCGAAGATTAATGACTG
TAAAAGATAATACTAAACTCTCACAACCACCTGAAGGGAGCACATTTTAA
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCTTAAAATGTGCTCCCTTCAGGTGGTTGTGAGAGTTTAGTATTAT
CTTTTACAGTCATTAATCTTCGCAGTTCTTGTAATAAAGACTTGATAGTA
TACTCTAGCATCATTTTCATCACAAACAAAGGTGATGGCTAAACCCTTTG
TGCCGAATCGCCCTGCACGTGCCACTCTGTGTAGATAAGTGTCAGAATCT
TCAGGCCGTTCATTTGAGTCAACGAAGTTATTAACTTTGTCTGCCAATTG
AAGTGCTAGCGCTTGAAGTCTAGGTGGITCAGATCGATGAAGGACCACTG
TTTGGG

The respective hpRNA sequences correspond to the following elements:

Nucleotides 1-100 and 607-706: Respective sense and reverse complement sequences of SEQ ID NO: 32, corresponding to nucleotides 761-860 of SEQ ID NO: 30, with T793C substitution to eliminate an Xba I site;

Nucleotides 101-200 and 507-606: Respective sense and reverse complement sequences of SEQ ID NO: 38, corresponding to nucleotides 462-561 of SEQ ID NO: 37;

Nucleotides 201-300 and 407-506: Respective sense and reverse complement sequences of SEQ ID NO: 56, corresponding to nucleotides 324-423 of SEQ ID NO: 55;

Nucleotides 301-406: 106 bp Loop fragment (SEQ ID NO: 61) based on Partial Leptocibe invasa Chitin Synthase intron.

The sense mRNA transcribed from construct 2 has the following sequence (SEQ ID NO: 65):

CCCAAACAGTGGTCCTTCATCGATCTGAACCACCTAGACTTCAAGCGCTA
GCACTTCAATTGGCAGACAAAGTTAATAACTTCGTTGACTCAAATGAACG
GCCTGAAGATTCTGACACTTATCTACACAGAGTGGCACGTGCAGGGCGAT
TCGGCACAAAGGGTTTAGCCATCACCTTTGTTTGTGATGAAAATGATGCT
AGAGTATACTATCAAGTCTTTATTACAAGAACTGCGAAGATTAATGACTG
TAAAAGATAATACTAAACTCTCACAACCACCTGAAGGGAGCACATTTTAA

### Silencing Construct 3

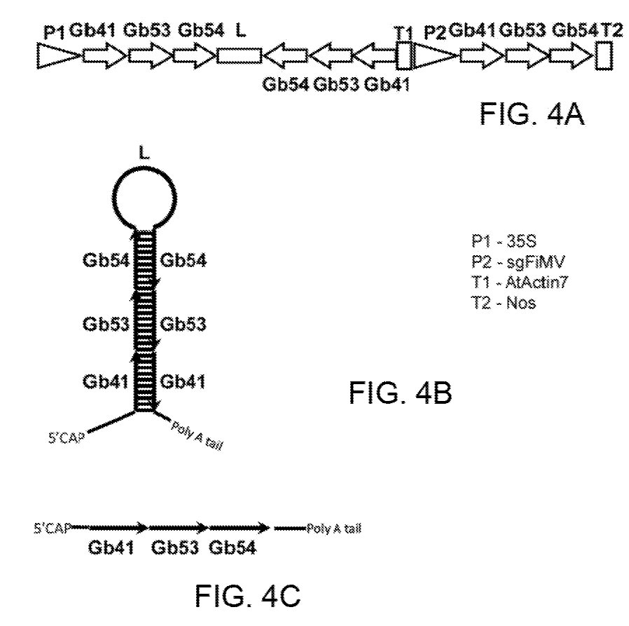

Silencing Construct #3 is shown schematically in FIG. 4. Respective 100 bp fragments of each of the Gb Mor gene (SEQ ID NO: 43), Trip 1 gene (SEQ ID NO: 49) and, tws gene (SEQ ID NO: 51), SEQ ID NO: 44, SEQ ID NO: 50 and SEQ ID NO: 52, respectively, were fused and synthesized in inverted repeats separated by 106 bp of a loop sequence (Loop 1; SEQ ID NO: 61). Transcription initiation was driven by the 35S CaMV promoter (SEQ ID NO: 57). Transcription termination was provided by the AtActin7 Terminator (SEQ ID NO: 59). The select 100 bp of SEQ ID NO: 43, 49 and 51 (respectively, SEQ ID NO: 44, SEQ ID NO: 50 and SEQ ID NO: 52) were synthesized in sense orientation between sgFIMV Promoter (SEQ ID NO: 58) to NOS Terminator (SEQ ID NO: 60).

Transcription of construct 3 would yield two mRNAs: (1) A hairpin RNA (hpRNA) with a stem formed by the reverse complementary sequences of the three Gb 100 bp sequences, to silence the corresponding Gb genes (see FIG. 4B); and (2) sense mRNA of the three, fused Gb genes (see FIG. 4C).

The hpRNA formed upon transcription of the hpRNA-forming transgene of Construct #3 has the following sequence (SEQ ID NO: 66):

AGATATTGTTGGATATGGATAAGAAACCAGATACGCTACTCAAGAAAGAA
GGCTCTGAGATCCCATCTAATTTTGGATTGAAATTAGACCAGTATGCTAA
TTACAGTAATGATGACACCATGGGAAATAAATGTTATCTCTCCGTTCTTG
ATGTTAGGACTACTGATGCTACAAATTCAGGAGACCCAGTTGTTAAGATG
TCTTCCGTATGTTTGATAGAATTAATAAACGAGATGCCACACTAGAGGCA
TCAAGGGAAATAGCAAAGCCTAAAACACTACTTAGACCTAGAAAAGTATG
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCCATACTTTTCTAGGTCTAAGTAGTGTTTTAGGCTTTGCTATTTC
CCTTGATGCCTCTAGTGTGGCATCTCGTTTATTAATTCTATCAAACATAC
GGAAGACATCTTAACAACTGGGTCTCCTGAATTTGTAGCATCAGTAGTCC
TAACATCAAGAACGGAGAGATAACATTTATTTCCCATGGTGTCATCATTA
CTGTAATTAGCATACTGGTCTAATTTCAATCCAAAATTAGATGGGATCTC
AGAGCCTTCTTTCTTGAGTAGCGTATCTGGTTTCTTATCCATATCCAACA
ATATCT

The respective hpRNA sequences correspond to the following elements:

Nucleotides 1-100 and 607-706: Respective sense and reverse complement sequences of SEQ ID NO: 44, corresponding to nucleotides 248-347 of SEQ ID NO: 43;

Nucleotides 101-200 and 507-606: Respective sense and reverse complement sequences of SEQ ID NO: 50, corresponding to nucleotides 333-432 of SEQ ID NO: 49;

Nucleotides 201-300 and 407-506: Respective sense and reverse complement sequences of SEQ ID NO: 52, corresponding to nucleotides 209-308 of SEQ ID NO: 51;

Nucleotides 301-406: 106 bp Loop fragment (SEQ ID NO: 61) based on Partial Leptocibe invasa Chitin Synthase intron.

The sense mRNA transcribed from construct 3 has the following sequence (SEQ ID NO: 67):

AGATATTGTTGGATATGGATAAGAAACCAGATACGCTACTCAAGAAAGAA
GGCTCTGAGATCCCATCTAATTTTGGATTGAAATTAGACCAGTATGCTAA
TTACAGTAATGATGACACCATGGGAAATAAATGTTATCTCTCCGTTCTTG
ATGTTAGGACTACTGATGCTACAAATTCAGGAGACCCAGTTGTTAAGATG
TCTTCCGTATGTTTGATAGAATTAATAAACGAGATGCCACACTAGAGGCA
TCAAGGGAAATAGCAAAGCCTAAAACACTACTTAGACCTAGAAAAGTATG

### Example 4

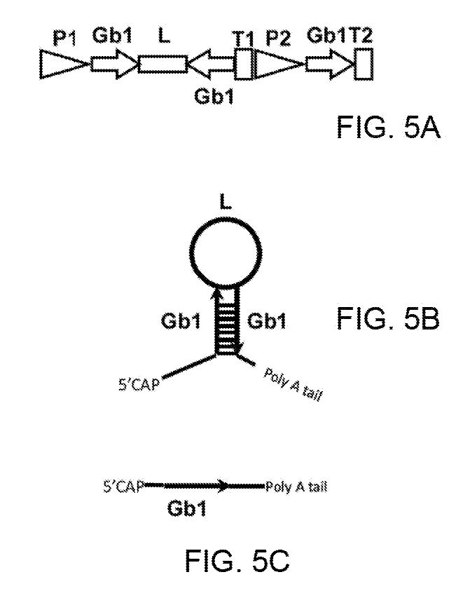

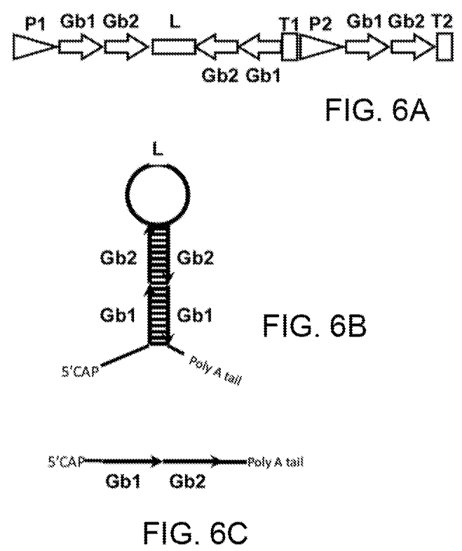

Schematic representations of silencing constructs comprising segments from one and two Gb genes are shown in FIG. 5 and FIG. 6, respectively. Silencing constructs contain two transgenes. A first transgene comprises fragments from each of one (see FIG. 5) or two (FIG. 6) Gb genes which are fused (in the case of constructs containing two Gb genes) and synthesized in inverted repeats, separated by a loop sequence. See FIGS. 5A and 6A. Transcription of this transgene (initiated at promoter P1 and terminated at T1) produces a hairpin RNA, containing a dsRNA section, formed by annealing of the inverted-repeat sequences of the respective Gb genes, and a loop region. See FIGS. 5B and 6B. A second transgene contains the Gb gene(s), oriented to be transcribed to yield a sense strand. See FIGS. 5C and 6C.

### Silencing Construct #4

Single gene control sequences are generated using a combination of sequences comprising a first sequence of 100 bp sense 100 bp (approximate) loop 100 bp antisense, where “100 bp sense” and “100 bp antisense” refer to complementary sequences from a target gene, and a second 100-bp sense amplifying sequence.

To construct silencing construct #4, 100 bp fragments of the Gb tws gene (SEQ ID NO: 51), i.e. SEQ ID NO: 52, were fused and synthesized in inverted repeats separated by 106 bp of a loop sequence (Loop 1; SEQ ID NO: 61). Transcription initiation was driven by the 35S CaMV promoter (SEQ TD NO: 57). Transcription termination was provided by the AtActin7 Terminator (SEQ ID NO: 59). The select 100 bp of Gb SEQ ID NO: 51 (i.e., SEQ ID NO: 52) were synthesized in sense orientation between sgFIMV Promoter (SEQ ID NO: 58) to NOS Terminator (SEQ ID NO: 60).

Transcription of construct 4 would yield two mRNAs: (1) A hairpin RNA (hpRNA) with a stem formed by the reverse complementary sequences of the Gb 100 bp sequences, to silence the corresponding Gb gene (see FIG. 5B); and (2) sense mRNA of the Gb gene (see FIG. 5C).

The hpRNA formed upon transcription of the hpRNA-forming transgene of Construct #4 has the following sequence:

(SEQ ID NO: 68)
TCTTCCGTATGTTTGATAGAATTAATAAACGAGATGCCACACTAGAGGCA
TCAAGGGAAATAGCAAAGCCTAAAACACTACTTAGACCTAGAAAAGTATG
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCCATACTTTTCTAGGTCTAAGTAGTGTTTTAGGCTTTGCTATTTC
CCTTGATGCCTCTAGTGTGGCATCTCGTTTATTAATTCTATCAAACATAC
GGAAGA

The respective hpRNA sequences correspond to the following elements: Nucleotides 1-100 and 207-306: Respective sense and reverse complement sequences of SEQ ID NO: 52, corresponding to nucleotides 209-308 of SEQ ID NO: 51; Nucleotides 101-206: 106 bp Loop fragment (SEQ ID NO: 61) based on Partial Leptocibe invasa Chitin Synthase intron.

The sense mRNA transcribed from construct 4 has the following sequence (SEQ ID NO: 52):

TCTTCCGTATGTTTGATAGAATTAATAAACGAGATGCCACACTAGAGGCA
TCAAGGGAAATAGCAAAGCCTAAAACACTACTTAGACCTAGAAAAGTATG

### Silencing Construct #5

Two gene control sequences are generated using a combination of sequences comprising a 100 bp sense sequence 1-100 bp sense sequence 2-100 bp (approximate) loop-100 bp antisense sequence 1-100 bp sense sequence 2 where “100 bp sense” and “100 bp antisense” refer to complementary sequences from a target gene, and a second 100-bp sense amplifying sequence.

To construct silencing construct #5, 100 bp fragments the Trip1 gene (SEQ ID NO: 49) and tws gene (SEQ ID NO: 51), SEQ ID NO: 50 and SEQ ID NO: 52, respectively, were fused and synthesized in inverted repeats separated by 106 bp of a loop sequence (Loop 1; SEQ ID NO: 61). Transcription initiation was driven by the 35S CaMV promoter (SEQ ID NO: 57). Transcription termination was provided by the AtActin7 Terminator (SEQ ID NO: 59). The select 100 bp of SEQ ID NO: 49 and 51 (respectively SEQ ID NO: 50 and SEQ ID NO: 52) were synthesized in sense orientation between sgFIMV Promoter (SEQ ID NO: 58) to NOS Terminator (SEQ ID NO: 60).

Transcription of construct 5 would yield two mRNAs: (1) A hairpin RNA (hpRNA) with a stem formed by the reverse complementary sequences of the Gb 100 bp sequences, to silence the corresponding Gb gene (see FIG. 6B); and (2) sense mRNA of the Gb gene (see FIG. 6C).

The hpRNA formed upon transcription of the hpRNA-forming transgene of Construct #5 has the following sequence:

(SEQ ID NO: 69)
TTACAGTAATGATGACACCATGGGAAATAAATGTTATCTCTCCGTTCTTG
ATGTTAGGACTACTGATGCTACAAATTCAGGAGACCCAGTTGTTAAGATG
TCTTCCGTATGTTTGATAGAATTAATAAACGAGATGCCACACTAGAGGCA
TCAAGGGAAATAGCAAAGCCTAAAACACTACTTAGACCTAGAAAAGTATG
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCCATACTTTTCTAGGTCTAAGTAGTGTTTTAGGCTTTGCTATTTC
CCTTGATGCCTCTAGTGTGGCATCTCGTTTATTAATTCTATCAAACATAC
GGAAGACATCTTAACAACTGGGTCTCCTGAATTTGTAGCATCAGTAGTCC
TAACATCAAGAACGGAGAGATAACATTTATTTCCCATGGTGTCATCATTA
CTGTAA

The respective hpRNA sequences correspond to the following elements:

Nucleotides 1-100 and 407-506: Respective sense and reverse complement sequences of SEQ ID NO: 50, corresponding to nucleotides 333-432 of SEQ ID NO: 49; Nucleotides 101-200 and 307-406: Respective sense and reverse complement sequences of SEQ ID NO: 52, corresponding to nucleotides 209-308 of SEQ ID NO: 51;

Nucleotides 201-306: 106 bp Loop fragment (SEQ ID NO: 61) based on Partial Leptocibe invasa Chitin Synthase intron.

The sense mRNA transcribed from construct 5 has the following sequence:

(SEQ ID NO: 70)
TTACAGTAATGATGACACCATGGGAAATAAATGTTATCTCTCCGTTCTTG
ATGTTAGGACTACTGATGCTACAAATTCAGGAGACCCAGTTGTTAAGATG
TCTTCCGTATGTTTGATAGAATTAATAAACGAGATGCCACACTAGAGGCA
TCAAGGGAAATAGCAAAGCCTAAAACACTACTTAGACCTAGAAAAGTATG

### Example 5

### Expression of RNAi Constructs in Eucalyptus

RNA constructs are transformed into eucalyptus using a protocol essentially described in Prakash et al., In Vitro Cell Dev Biol.-Plant, 2009, 45:429-434. Briefly, shoots of Eucalyptus are propagated in vitro on Murashige and Skoog (MS) basal salt medium consisting of 3% (w/v) sucrose and 0.8% (w/v) agar. All in vitro plant materials are incubated at 25±2° C. under a 16-h photoperiod with cool white fluorescent lamps with an intensity of 30 μEm⁻² s⁻¹. A. tumefaciens strain LBA 4404 harboring a binary vector pBI121 containing nptII gene is used for transformation. Bacterial culture collected at late log phase are pelleted and resuspended in MS basal salt medium. Leaves from in vitro material are collected and used as explants for transformation experiments.

Explants are precultured on the MS regeneration medium supplemented with 0.5 mg/l BAP and 0.1 mg/l NAA for 2 d. Precultured leaf explants are gently shaken in the bacterial suspension for 10 min and blotted dry on a sterile filter paper. Explants are then cocultivated in medium under the preculture conditions for 2 d. Following cocultivation, explants are washed in MS liquid medium, blotted dry on a sterile filter paper, and transferred to MS regeneration medium containing 0.5 mg/l BAP and 0.1 mg/l NAA supplemented with 40 mg/l kanamycin and 300 mg/l cefotaxime. After 4-5 weeks of culture, regeneration is observed and explants are transferred to liquid elongation medium (MS medium supplemented with 0.5 mg/l BAP, 40 mg/l kanamycin, and 300 mg/l cefotaxime) on paper bridges. The elongated shoots (1.5-2 cm) are propagated on MS medium with 0.1 mg/l BAP. Leaf segments are regenerated and elongated shoots are analyzed by PCR and western blot. Positive shoots are multiplied to 10 copies on MS medium containing 0.04 mg/L BAP. A few leaves are excised from the shoots and analyzed by RT-PCR.

Expression of dsRNAs is measured using RT-PCR. Total RNA from 50 mg fresh transgenic plant tissue was purified using EPICENTRE MasterPure™ Plant RNA Purification Kit (Cat. #MPRO9010) following by DNAse treatment with Ambion TURBO DNA-Free™ Dnase (Cat. #AM1907). 1 μl of total RNA from each sample is analyzed by RT PCR. RT PCR is performed using Invitrogen SuperScript III One-Step RT-PCR System with Platinum Taq DNA Polymerase kit (Cat. #12574-018). As a control, the Platinum Taq DNA Polymerase kit (Cat. #12574-018 and #10966-018) is used to recognize traces of DNA contaminations. No fragment amplification is expected for this control.

To detect expression of RNA from constructs, RT-PCR is prepared using primer pairs that generate fragments indicative of the presence and expression of Gb transgenes.

### Example 6

Bioassay of Gb dsRNA Constructs

### Sup Suckers Artificial Feeding

100 μl of feeding solution (standard diet described in Febvay et al., Canadian Journal of Zoology 66:2449-2453, 1988) is placed between two stretched paraffin membranes on a plastic cap. Gb nymphs and/or adults are placed on the paraffin membranes and covered with a Petri dish lid that is ventilated by a 1 cm hole covered with a mesh. Feeding solution containing siRNA, and/or dsRNA and/or hpRNA and/or microRNA homologous to one or more of the target genes described above in Table 1 is provided. RNA concentration can be between 10 ng to 500 ng per microliter. Psyllids are incubated for up to 40 days. Data on the number viable and dead bugs data is compiled daily. Candidate lethal sequences and their corresponding lethal target genes are ranked based on live to dead bug ratios data.

### Example 7

Test of Protective Effect of Gb dsRNA Constructs

Eucalyptus plants are transformed with plasmids comprising construct 1, construct 2 or construct 3 and transgenic lines are established. Controls lines are established by transforming plants with vector alone, without insertion of Gb nucleic acids or without nucleic acids that could form siRNAs.

Transgenic, wt, and control eucalyptus plants are grown in insect proof cages in the greenhouse together with nymph and/or adult Gb. The insect proof cages keep the inoculums in while preventing outside pests from entering the cage. Following Gb inoculation, the appearance of lerps, which compete with the plant for photosynthesis products, plant synthesized sugars, is evaluated. Low infestation leads to growth inhibition while high infestation can cause cessation of tree growth and death. Lerps can be seen on the upper or lower surface of the leaves. Plants are examined to determine the number of Gb eggs and clusters of eggs on the plant tissues including leaves, reproductive organs, branches, stems, but predominantly on the leaves, and the number of dead or dysfunctional Gb specimens found on or adjacent to the plants. The primary endpoints for a resistant plant can be either lack of symptoms, lack of viable pests on the plant surfaces and/or lack of eggs or egg clusters on the plants or retarded or altered growth development of nymphs. In some cases resistant plants may simply cause the contacting pests to become unviable or sterile without causing pest death. Five independent transformation events of transgenic eucalyptus plants transcribing dsRNA are tested. Ten lines of each transformation event are inoculated with adult Gb in 3 independent repeats. Number of vital Gb adults, their size, eggs, clusters of eggs, nymphs, dead bugs are recorded every day for 40 days after inoculation.

Exemplary prophetic result: Transgenic plants transcribing dsRNA targeting Gb genes exhibit fewer symptoms, fewer vital Gb specimens, less eggs and less egg clusters, and/or less newly hatched nymphs, compared to controls. Transgenic plant lines are resistant to Gb infection showing less plant growth inhibition, less leaf and other tissue damage fewer lerps and adults compared to control and wt plants that are infected with Gb.

### Bioassay:

### Whole Plant Assay:

Five 3 month old transgenic and wt eucalyptus plants of each line were grown in a green house at 24° C., 40-60% RH and 16 hr of light per day. The trees were tested for Gb resistance for a period of 40 days, from tree age of 3 months. Each plant line was maintained in a separate insect proof cage and each plant was inoculated with 50 adult and/or nymphs bugs that were reared in culture.

Every day after inoculation the following parameters were tested:

1. Number of live bugs on each plant.
2. Number of live bugs not on plants.
3. Number of dead bugs.
4. Number of deformed, dysfunctional or non-reproductive pests.
5. Number of eggs laid.
6. Number of nymph hatched.
7. Number of lerps.
8. Number of defoliated leaves.
9. Number of dead branches.
10. Number of dead plants.

### Single Leaf Assay:

Five 3 months old transgenic and wt eucalyptus of each line are grown in a green house at 24° C., 40-60% RH and 16 hr of light a day. The trees are tested, from age 3 months, for Gb resistance for a period of 40 days. Each line is contained in a separate insect proof cage and 5 leaves of each plant are covered with clip-on insect cages described by University of Arizona Center for Insect Science Center for Education Outreach http://insected.arizona.eduigg/resource/clip.html. Ten adult insects are placed inside each leaf clip cage. Clip cages can be clipped over a leaf-feeding insect without disturbing the insect or the plant. These cages provide a simple way to isolate one or more sap-sucking pests or other small insects for investigation and observation.

Every day the following observations were made:
1. Percent mortality ((total number of insects−live insects)/total number of insects)×100 is calculated.
2. Extent, number and percentage of discolored leaves are recorded.
3. Number of eggs or egg clusters

### Results:

### Full Plant Assay:

Transgenic eucalyptus will significantly differ from the wt in these parameters:

1. Fewer vital insects on the plants.

2. More live insects off plants.

3. More dead insects.

4. Fewer eggs and/or egg clusters laid.

5. Fewer nymphs hatched.

6. Fewer defoliated leaves.

7. Fewer discolored leaves.

8. Fewer dead branches

9. Fewer dead plants.

Transgenic trees can have part or all of the above list as a phenotype following Gb infestation.

### Single Leaf Assay, Predicted Results:

Higher mortality rate is observed in the cages set around transgenic leaves compared to wild type starting day 2 and onwards.

No lerps are visible on the transgenic leaves for the whole infection period.

No eggs or egg clusters are found on leaves of transgenic plants compared to wild type.

A number of embodiments of the invention have been described. Nevertheless, it will be understood that various modifications may be made without departing from the spirit and scope of the invention. Accordingly, other embodiments are within the scope of the following claims.

All patents, patent publications and non-patent literature referenced in the specification are hereby incorporated herein by reference in their entireties.

### REFERENCES

1. Baum J A, et al., Control of coleopteran insect pests through RNA interference, (2007), Nat Biotechnol. 25:1322-6.
2. Frizzi A, et al., Tapping RNA silencing pathways for plant biotechnology, (2010), Plant Biotechnol 8:655-77.
3. Gordon K H, et al., RNAi for insect-proof plants, (2007), Nat Biotechnol 25:1231-2.
4. Huvenne H, et al., Mechanisms of dsRNA uptake in insects and potential of RNAi for pest control: a review, (2010), J Insect Physiol 56:227-35.
5. Mao Y B, et al., Silencing a cotton bollworm P450 monooxygenase gene by plant-mediated RNAi impairs larval tolerance of gossypol, (2007), Nat Biotechnol 25:1307-13.
7. Nunes F M, et al., A non-invasive method for silencing gene transcription in honeybees maintained under natural conditions, (2009), Insect Biochem Mol Biol 39:157-60.
8. Price D R, et al., RNAi-mediated crop protection against insects, (2008), Trends Biotechnol 26:393-400.
10. Tinoco M L, et al., In vivo trans-specific gene silencing in fungal cells by in planta expression of a double-stranded RNA, (2010), BMC Biol 8:27.
11. Hannon, G. J., RNA interference, (2002), Nature 418:244-251
12. Baulcombe, D., RNA silencing in plants, (2004), Nature 431:356-363.
13. Pei Y, et al., On the art of identifying effective and specific siRNAs, (2006), Nature Methods 3(9):670-676.
14. Cullen, BR., Enhancing and confirming the specificity of RNAi experiments, (2006), Nature Methods 3(9):677-681.
15. Chen et. al, New Genes in Drosophila Quickly become essential, Science (2010), 330:1682-5.
16. Dietzl et. al., A Genome Wide Transgenic RNAi Library for Conditional Gene Inactivation in Drosophila. Nature (2007), 448:151-7.

SEQUENCES

Gene #3
Blw
ATP synthase subunit alpha (partial)
SEQ ID NO: 1
AACTTGGAACCTGACAATGTTGGTGTTGTAGTATTCGGTAATGATAGATT
AATCAAGGAAGGAGACATTGTCAAACGTACTGGTGCTATTGTTGATGTAC
CTGTTGGTGAAGATTTGTTGGGAAGAGTAGTTGATGCTTTAGGTAACACC
ATTGATGGAAAAGGACCACTCACCTCTAAAACTCGTTTCCGTGTTGGAAT
CAAGGCCCCTGGAATCATTCCCCGTATTTCTGTAAGAGAACCTATGCAAT
CTGGTATTAAAGCTGTAGATTCCTTGGTACCAATTGGTCGTGGTCAACGT
GAGTTGATCATTGGAGATCGTCAAACTGGAAAAACTGCTTTGGCCATTGA
TACCATCATCAACCAAAAGAGATTCAATGACTCTGATGACGAAAAGAAAA
AGTTGTACTGTATCTATGTTGCTATTGGTCAAAAGAGATCTACTGTAGCT
CAAATCGTAAAACGTTTAACTGACTCTGGTGCCATGAAATACACCATCAT
TGTATCAGCTACCGCCTCTGATGCTGCCCCICTACAATACTTGGCTCCTT
ACTCTGGATGTGCCATGGGAGAATTCTTCCGTGACAATGGAAAACACGCC
TTGATCATCTTTGACGATTTATCAAAACAAGCTGTTGCTTACCGTCAAAT
GTCTCTGCTGCTTCGTCGTCCCCCAGGTCGTGAAGCTTACCCCGGAGATG
TATTTTATCTTCACTCTCGTCTTCTTGAACGTTCCGCTAAAATGTCTGAA
GCCCATGGAGGTGGTTCTTTAACTGCTTTACCCGTTATTGAAACTCAAGC
TGGAGATGTATCAGCTTATATCCCAACCAATGTAATTTCTATTACTGATG
GACAAATTTTCTTGGAAACTGAATTGTTCTACAAAGGTATTCGTCCCGCT
ATCAACGTAGGATTGTCTGTATCCCGTGTAGGATCTGCTGCCCAAACCAG
AGCCATGAAACAGGTTGCCGGTTCAATGAAATTGGAATTGGCCCAATACC
GTGAAGTCGCCGCTTTTGCCCAATTCGGTTCTGATTTAGATGCTGCTACC
CAACAATTGCTAAACCGTGGTGTTCGTTTGACTGAATTGTTGAAACAAGG
TCAATATGTACCAATGGCTATTGAAGAACAAGTTGCTGTCATCTACTGTG
GTGTCCGTGGTCACTTGGACAAATTAGACCCAGCAAAGATCACCACTTTT
GAAAAAGAATTCTTAGCTCACATTAAAACTTCCGAAAAAGCTTTATTGGA
AAGTATCAAGAAAGAAGGAAAAATCACTGAAGATACCGATGCTAAGTTGA
AGACTGTTGTACAGAACTTCCTTGCTAACTTCACTGGTTAG
Gene #3
Nucleotides 460-559 of SEQ ID NO: 1
Blw
ATP synthase subunit alpha
SEQ ID NO: 2
AAACGTTTAACTGACTCTGGTGCCATGAAATACACCATCATTGTATCAGC
TACCGCCTCTGATGCTGCCCCTCTACAATACTTGGCTCCTTACTCTGGAT
Gene #7
Pros28.1A
proteasome subunit alpha type-like (full)
SEQ ID NO: 3
ATGAGTAGCTCCAGATATGACCGGGCCATCACTGTGTTCTCTCCTGATGG
TCATTTACTTCAAGTTGAATATGCCCAAGAAGCTGTCAGAAAAGGATCAA
CTGCTGTTGGAGTCCGTGGAGACAATGTTGTAGTTCTGGGTGTTGAAAAG
AAATCAGTGGCAAAATTACAAGAAGAAAGAACTGTTAGAAAAATATGTTT
ACTTGATGATCATGTTGTCATGGCATTTGCTGGTTTGACAGCTGATGCTC
GTATATTAATTAATCGTGCACAAATTGAATGTCAGTCTCACAAATTGACT
GTTGAAGATCCTGTTACATTAGAATATATTACTAGGTATATTGCTGGTTT
GAAGCAAAAATATACTCAAAGTAATGGAAGAAGACCTTTTGGTATATCAT
GTTTGATTGGAGGATTTGATTATGATGGAAAAGCAAGACTATATCAAACT
GAACCTTCTGGCATTTATTATGAATGGAAGGCTAATGCAACAGGAAGAAG
TGCTAAGACAGTTCGTGAATTCTTAGAGAAATATTATAAAGCTGAAGAAC
TAACCACAGAAAAGGCTACAGTTAAATTAGCAATACGGGCCTTACTAGAA
GTAGTACAATCTGGACAAAAGAATCTAGAAATTGCTGTCATGAGGCATGG
AAAGCCTATGGAGATGTTGACTGCAGCTAAAATAGAAGAATATGTTATTG
AAATTGAAAAAGAAAAGGAAGAAGAAGCAGAAAAGAAAAAGCAAAAGAAA
TAG
Gene #7
Nucleotides 608-707 of SEQ ID NO: 3
Pros28.1A
proteasome subunit alpha type-like
SEQ ID NO: 4
AATCTGGACAAAAGAATCTAGAAATTGCTGTCATGAGGCATGGAAAGCCT
ATGGAGATGTTGACTGCAGCTAAAATAGAAGAATATGTTATTGAAATTGA
Gene #8
Prosα3T
proteasome subunit alpha type-like (full)
SEQ ID NO: 5
ATGGCTAGGAGATATGATTCACGTACAACAATCTTCTCCCCAGAGGGACG
ATTGTATCAAGTAGAATATGCTATGGAAGCTATCAGTCATGCTGGTACTT
GTTTGGGTATCCTAGCTAATGATGGCATTCTGCTGGCAGCCGAGAAAAGA
AACACCAATAAATTACTAGATGAAGGAAATTCATCTGAGAAAATTTACAA
GTTGAATGATAATATGGTTTGCAGTGTAGCTGGTATTACTTCAGATGCTA
ATGTTCTAACATCAGAATTGAGACTGATAGCTCAACGTTATTTAATTCAA
TATGATGAACCCATACCTTGTGAACAACTGGTATCTTGGTTATGTGATAT
CAAACAAGGATATACTCAATATGGAGGAAAAAGACCGTTTGGTGTATCAA
TTCTGTATATGGGTTGGGATAAACAGTATGGCTACCAATTATATCAATCA
GATCCTAGTGGAAACTACAGTGGATGGAAAGCAACATGTATTGGAAATAA
TAGTGCAGCTGCTATTTCTAATTTGAAACAAGAGTATAAGGAAGATTTGA
CTTTAGATAATGCCAAGCTTTTAGCTATCAAAGTTCTCAGTAAAATATTG
GATATGACAAAACTAACTCCGGAGAAAGTTGAACTGGCAACACTTACAAG
AAAAGATGGCAAAACTTTTACTAAAATTTTATCAGCAAACGAAGTTGAAG
CTTTGATCGCTGCTCATGAGAAAGCAGAAAGTTTAGAAAAAGAGAAAGAA
AAACAAGCAAAGGCTGCTGCTGCTAGCTCTTCTTCTTAG
Gene #8
Nucleotides 690-789 of SEQ ID NO: 5
Prosα3T
proteasome subunit alpha type-like
SEQ ID NO: 6
CGAAGTTGAAGCTTTGATCGCTGCTCATGAGAAAGCAGAAAGTTTAGAAA
AAGAGAAAGAAAAACAAGCAAAGGCTGCTGCTGCTAGCTCTTCTTCTTAG
Gene #9
CG2931
RNA-binding protein 42-like (partial)
SEQ ID NO: 7
CGAACAGCTGGTGGCACTGTTTGGGAAGATCCAACACTTCTTGAATGGGA
AGATGATGATTTTCGACTGTTTTGTGGAGATTTAGGAAATGACGTCACAG
ATGAATTACTAATTAGAACCTTTTCAAAATATCCTTCATTTTTAAAGGCC
AAAGTTGTTCGAGATAAAAGAACAAATAAAACAAAAGGTTTTGGATTTGT
TAGTTTTAAAGATCCTCAAGATTTTATTCGTGCAAATAAAGAAATGAATG
GAAGATATGTTGGTAGTCGCCCTATTAAACTAAGAAAAAGTAATTGGAGA
AACCGAAGTTTAGAAGTTGTAGAAAAAAGAGAAGAAAAAGCAACTCTGAT
TGGTCTGCTCACAGGT
Gene #9
Nucleotides 267-366 of SEQ ID NO: 7
CG2931
RNA-binding protein 42-like
SEQ ID NO: 8
TCGCCCTATTAAACTAAGAAAAAGTAATTGGAGAAACCGAAGTTTAGAAG
TTGTAGAAAAAAGAGAAGAAAAAGCAACTCTGATTGGTCTGCTCACAGGT
Gene #10
CG31524
prolyl 4-hydroxylase subunit alpha-2-like isoform
2 (partial)
SEQ ID NO: 9
GAAGCCTACTTAGTGCCACGAATTGTCCTCTACAGAGATGTCATGTATGA
CTCTGAAATTGATCTTATCAAGAAAATGGCTCAACCTAGACTTCGTAGAG
CAACAGTACAAAATTATAAAACTGGAGAGTTAGAAATTGCAAATTATAGA
ATCAGCAAATCAGCATGGTTAAGAGAACCAGAACATCCAGTTGTAGAAAG
AATCAGCAGAAGAGTTGAAGATATGACTGGACTTACCACTGAAACTGCTG
AAGAACTTCAAGTTGTTAACTATGGAATTGGTGGTCACTATGAACCTCAT
TATGACTTTGCCAGGCCTGGTGAAGCCAACGCATTCAAATCTTTAGGAAC
TGGCAACAGAGTAGCAACAGTATTATTTTATATGAGTGATGTATCTCAGG
GAGGGGCAACAGTTTTTACTTCTTTAAATTTATCATTGTGGCCAGAAAAA
GGAACTGCAGCTTTTTGGCACAATCTTCACTCAAGTGGGGACGGAAATTA
TCTAACTAGACATGCTGCTTGTCCAGTTCTTACAGGATCAAAATGGGTAT
CAAACAAATGG
Gene #10
Nucleotides 1-100 of SEQ ID NO: 9
CG31524
prolyl 4-hydroxylase subunit alpha-2-like
isoform 2
SEQ ID NO: 10
GAAGCCTACTTAGTGCCACGAATTGTCCTCTACAGAGATGTCATGTATGA
CTCTGAAATTGATCTTATCAAGAAAATGGCTCAACCTAGACTTCGTAGAG
Gene #12
CG3590
adenylosuccinate lyase-like (partial)
SEQ ID NO: 11
AAAGGAACAACTGGTACTCAAGCTTCTTTTATGGAACTTTTTAATGGAGA
TGGCGAAAAGGTGAAAGCTTTAGACCGTCTGGTTACCAAAAAAGCTGGTT
TCTCTACTTCTCACATCATCTGTGGCCAAACATACTCTAGAAAAGTTGAC
GTCATCGTAACGGGAGCTCTCAGCAGTCTAGGTGCCACAATTCACAAGCT
TGCAACAGATTTACGTTTGTTAGCACATATGAAAGAAGTTGAAGAGCCTT
TTGAATCAACTCAAATTGGTTCCAGTGCAATGGCCTATAAAAGGAACCCT
ATGAGAAGTGAGAGACTGTGTTCTTTAGCAAGATTCCTAATGAGTTTACA
TCAAAACTCATTGAACACTGCCAGTACACAGTGGATGGAACGTACTCTTG
ATGATAGTGCTAACAGGAGACTTACTCTATCCGAATCATTCCTCACCGCA
GACTGCCTTTTAATGACCCTTCAAAATGTTTTAGAAGGATTAGTAGTTAA
TAAAAAGTTATTCAGCGTCACATTGATAC
Gene #12
Nucleotides 66-185 of SEQ ID NO: 11
CG3590
adenylosuccinate lyase-like
SEQ ID NO: 12
AGCTTTAGACCGTCTGGTTACCAAAAAAGCTGGTTTCTCTACTTCTCACA
TCATCTGTGGCCAAACATACTCTAGAAAAGTTGACGTCATCGTAACGGGA
Gene #12
Nucleotides 66-165 of SEQ ID NO: 11 with T□C
substitution at nucleotide 136 of SEQ ID NO: 11,
to eliminate Xba I site
CG3590
adenylosuccinate lyase-like
SEQ ID NO: 13
AGCTTTAGACCGTCTGGTTACCAAAAAAGCTGGTTTCTCTACTTCTCACA
TCATCTGTGGCCAAACATACCCTAGAAAAGTTGACGTCATCGTAACGGGA
Gene #13
CG5451
WD40 repeat-containing protein SMU1-like
isoform 1 (partial)
SEQ ID NO: 14
AAATCACATGTAGAATGTGCCCGGTTTTCACCAGATGGTCAATATTTAAT
CACAGGTTCAGTAGATGGATTTATTGAAGTTTGGAATTTTACAACTGGAA
AAATCAGAAAAGATTTGAAATATCAAGCTCAGGATAACTTCATGTTGATG
GAAGAAGCCGTCATGTCTCTTGCTTACTCCAGAGATTCCGAAATGTTAGC
AAGTGGATCTCAGAGTGGAAAGGTCAAAGTTTGGAAAATAGCCACTGGAC
AATGTTTGAGGAAACTAGAAAAGGCACATTCTTTAGGTGTTACCTGTATT
CAATTTTCAAGAGACAACAGTCAAGTGTTGACAGCATCTTTTGACACATG
TGTCAGGATACATGGGCTGAAATCTGGAAAACTTCTAAAAGAATTTCGAG
GTCATACATCATTTGTGAATGACATATCTTTTACAGCAGATGGACATAAC
ATATTGAGTGCGTCTAGTGATGGTACAGTAAAAATGTGGAACATCAAAAC
AACAGAATGTACAAACACATTCAAGTCAATTGGAGCTAGTGATAAATCAG
TTAACAGTATTCACATACTTCCTAAGAATAATGAACATTTTGTTGTATGT
AACAAAACAAACACTGTTGTCATCATGAATATGCAAGGCCAAATTGTACG
ATCTTTGTCTTCTGGTAAAAGAGAAGGAGGAGACTTCCTATGTTGTACAA
TTTCCCCTCGTGGTGAATGGATCTATTGTGTTGGAGAAGATATGGTGTTA
TATTGTTTCTCAATTACATCTGGAAAATTGGAGCGTACTCTCAATGTTCA
CGAGAAATTAGTAATTGGATTGACTCATCACCCTCATCAAAATCTCCTAG
GAACCTACAGTGAAGATGGACTGCTCCGATTGTGGAAACCTTAA
Gene #13
Nucleotides 759-858 of SEQ ID NO: 14
CG5451
WD40 repeat-containing protein SMU1-like isoform 1
SEQ ID NO: 15
CTCAATTACATCTGGAAAATTGGAGCGTACTCTCAATGTTCACGAGAAAT
TAGTAATTGGATTGACTCATCACCCTCATCAAAATCTCCTAGGAACCTAC
Gene #21
dlg1
disks large 1 tumor suppressor protein-like
(partial)
SEQ ID NO: 16
GATCTCAAACAACAGATGTCACAAATTTCATCTACTGGAACCATATTAAG
AACATCTCAAAAGAGGTCGCTTTATGTAAGAGCTTTATTTGATTATGATC
CCACCAAAGATGATGGATTACCATCTCGAGGATTACCTTTCCATTATGGA
GATATTCTTCATGTAACCAATGCAAGTGATGATGAATGGTGGCAAGCTCG
TCGTGTTCTACCTTCTGGTGATGAACAAGGAATTGGTATTGTTCCTTCTA
AGAAACGTTGGGAAAGAAAACAAAGGGCACGAGATCGAACGGTCAAGTTT
CAAGGTCATGTACCAGTTTTATTAGAAAAGACATCAACGTTAGAAAGAAA
AAAGAAGAACTTCTCATTCAGTCGAAAGTTTCCATTCATGAAAAGTAAAG
ATGATAAATCTGAAGATGGTTCTGACCAAGAACCATTCATGTTATGTTAC
ACCCAAGACGATCCAACCACAGAAGGTACTGAAGAAGGTGTACTGTCCTA
TGAACCTGTCACTCAACTGCAGATAGAATACTCAAGGCCTGTTATTATAC
TTGGACCTTTGAAAGATAGAATTAATGATGATTTAATATCGGAGTTTCCT
GAAGAGTTTGGATCATGTGTACCACATACCACCAGAGCTAAAAGAGATTA
TGAAGTTGATGGAAGAGATTACCATTTTGTTGCATCAAGAGAACAAATGG
AAAAGGATATTCAAAACCATCTATTTATTGAAGCAGGACAATATAATGAT
AACCTATATGGAACTTCAGTGGCATCTGTCAGAGACGTTGCTGAAAGTGG
GAAGCATTGTATTTTAGATGTTAGTGGAAATGCCATCAAAAGACTTCAAG
TAGCACAGCTTTATCCTATTGCAATATTTATAAAACCAAAATCTGTTGAA
TCTATAATGGAAATGAATAAACGAATGACTGAAGAGCAAGCAAAGAAATT
ATATGACCGTGCCATGAAAATGGAACAGGAGTTTGGTGAATTTTTCACTG
CTGTTGTTCAAGGAGATATGCCAGAAGATATTTACCATAATGTGAAAGCA
GTCATCAAGGAACAGTCTGGACCTTCAATTTGGGTCCCTTCAAAAGATCC
TCTGTAG
Gene #21
Nucleotides 230-329 of SEQ ID NO: 16
dlg1
disks large 1 tumor suppressor protein-like
SEQ ID NO: 17
GAATTGGTATTGTTCCTTCTAAGAAACGTTGGGAAAGAAAACAAAGGGCA
CGAGATCGAACGGTCAAGTTTCAAGGTCATGTACCAGTTTTATTAGAAAA
Gene #24
e(r)
enhancer of rudimentary (partial)
SEQ ID NO: 18
ATGGCTCATACAATATTGCTAATTCAACCTGGTGTTAAGCCAGAGACTCG
AACATTTTCAGATTATGAATCTGTTAATGAGTGTATGGAAGGTGTCTGCA
AAATTTATGAGGAACATTTGAAAAGAATGAATCCCAACACTCCATCCATC
ACCTATGATATCAGTCAGTTGTTTGATTTTATTGACCAGTTGTCAGACCT
TTCATGTCTAGTTTATCAAAAGGGTTCCAACACG
Gene #24
Nucleotides 16-63 of SEQ ID NO: 18
e(r)
enhancer of rudimentary
SEQ ID NO: 19
TTGCTAATTCAACCTGGTGTTAAGCCAGAGACTCGAACATTTTCAGAT
Gene #26
ebi
f-box-like/WD repeat-containing protein ebi-like
(partial)
SEQ ID NO: 20
TGGCAAAGCAACAATTCGTTTGCCTCATGCTCAACTGACCAACATATTCA
TGTTTGTAAACTCCATTCTGACAAACCAATCAAAAGTTTTGAAGGCCACA
CGAATGAAGTGAATGCCATCAAATGGGACCCCCAAGGAATTCTTTGGTCT
TCTTGTTCTGATGATATGACATTAAAAATTTGGTCTCTTGATAAAGATGT
ATGTGTCCATGATCTGCAAGCACATAATAAAGAAATCTATACTATTAAAT
GGTCTCCAACCGGGCTCGAAACAGCCAATCCCAACATGAATTTGGTGCTA
GCCAGTGCTTCCTTTGACTCTACAGTCAGACTGTGGGATGTGGAAAGAGG
AGAATGTTTAAATACATTGACAAGGCACACAGGGGATAGG
Gene #26
Nucleotides 124-223 of SEQ ID NO: 20
ebi
f-box-like/WD repeat-containing protein ebi-like
SEQ ID NO: 21
TGGGACCCCCAAGGAATTCTTTGGTCTTCTTGTTCTGATGATATGACATT
AAAAATTTGGTCTCTTGATAAAGATGTATGTGTCCATGATCTGCAAGCAC
Gene #27
EcR
ecdysone receptor isoform A (partial)
SEQ ID NO: 22
ATTGTAGAATTTGCTAAGAGGTTACCTGGTTTCGACAAATTAGTTAGGGA
AGATCAAATTTCATTACTTAAGGCTTGTTCGAGTGAAGTAATGATGTTAC
GAATGGCAAGGAGGTATGATGCTCCTTCTGATTCGATATTGTTTGCAAAT
AACCAACCATATACTAGGGAGGCATACAAGTCTGCCGATATGGGAGAAAC
AGTAGATGATCTGCTCAAATTTTGTAGGCTTATGTATTCAATGAAAGTTG
ACAATGCAGAATATGCGTTGCTGACAGCCATTGTTATATTT
Gene #27
Nucleotides 130-229 of SEQ ID NO: 22
EcR
ecdysone receptor isoform A
SEQ ID NO: 23
GATTCGATATTGTTTGCAAATAACCAACCATATACTAGGGAGGCATACAA
GTCTGCCGATATGGGAGAAACAGTAGATGATCTGCTCAAATTTTGTAGGC
Gene #28
Ef1alpha48D
elongation factor 1-alpha-like (full)
SEQ ID NO: 24
ATGGGTAAAGAAAAGATTCATATTAACATTGTCGTTATTGGACATGTCGA
CTCCGGCAAGTCTACTACTACTGGACATTTGATCTACAAATGTGGAGGTA
TTGACAAACGTACCATTGAAAAGTTCGAGAAAGAAGCTCAAGAAATGGGT
AAAGGATCATTCAAATATGCCTGGGTACTTGACAAGCTCAAGGCTGAACG
TGAACGTGGTATCACCATTGATATTGCTCTGTGGAAGTTTGAAACAGCCA
AATACTATGTCACCATTATTGATGCCCCAGGACACAGAGATTTCATCAAA
AACATGATCACTGGAACATCTCAGGCTGATTGTGCTGTATTGATCGTAGC
TGCTGGTACTGGAGAATTTGAAGCTGGTATTTCCAAGAATGGTCAAACTC
GTGAACATGCTCTCCTTGCTTTCACCTTAGGAGTCAAACAATTGATTGTT
GGAGTCAACAAAATGGATTCTACTGAACCACCATACAGTGAGTCACGTTT
TGAGGAAATCAAGAAAGAAGTTAGTGGTTACATCAAGAAAATTGGTTACA
ATCCAGCTACAGTTGCATTTGTACCTATCTCAGGATGGCATGGAGACAAC
ATGTTGGAACCATCTGACAAGATGCCATGGTTCAAGGGCTGGGCTATTGA
ACGTAAAGAAGGAAAGGCTGATGGAAAATGTTTGATTGAAGCTTTAGATG
CAATTCTTCCCCCTAGTAGACCAACTGAAAAACCCCTGCGTTTACCATTG
CAGGACGTGTACAAAATTGGAGGTATTGGAACAGTACCAGTTGGTCGTGT
TGAAACTGGAGTATTGAAACCTGGTATGGTTGTCACCTTTGCCCCTGCCA
ACTTAACCACTGAAGTTAAATCCGTAGAAATGCACCACGAAGCTCTTCAA
GAAGCAGTTCCAGGAGACAATGTTGGTTTCAACGTAAAGAACGTCTCAGT
TAAAGAATTACGTCGTGGATTTGTTGCTGGAGATTCCAAGTCCAACCCAC
CCAAGGCTACCCAAGATTTCACAGCCCAAGTCATTGTATTGAACCACCCT
GGTCAAATTTCAAACGGTTATACTCCTGTACTTGATTGTCACACAGCTCA
CATTGCTTGTAAATTCTCTGAGATCAAAGAAAAGTGTGACCGTCGTACTG
GTAAAACTACTGAAGAAAATCCCAAATCAGTCAAATCTGGTGATGCTGCC
ATTGTAGTCCTTGTCCCATCTAAACCTATGTGTGTAGAATCTTTCTCTGA
CTTCCCTCCCTTGGGACGTTTTGCTGTCCGTGACATGAGACAAACTGTTG
CCGTCGGTGTTATCAAGAGTGTAAATTATAAAGATTTATCTGCTGGTAAA
GTAACAAAGGCTGCTGAAAAAGCTGCAAAGAAGAAATAA
Gene #28
Nucleotides 969-1038 of SEQ ID NO: 24
Ef1alpha48D
elongation factor 1-alpha-like
SEQ ID NO: 25
ATTTGTTGCTGGAGATTCCAAGTCCAACCCACCCAAGGCTACCCAAGATT
TCACAGCCCAAGTCATTGTA
Gene #29
Tef
Ef1gamma (partial)
SEQ ID NO: 26
TCTGGAACTTTATATTCGTGGCCCGAGAACTTCCGCACATATCAAATCCT
TGTAAGTGCAGAATACTCTGGATTTAAAGTGAATATACCTAAGGATTTTG
TATTCGGCAAATCAAACAAAAGTCCTGAGTTTGTAACGAAATTTTCGTCA
CCAAAGGTTCCAGCTTTTGAAGGTGCAGATGGCACTATTCTTACATCTAG
TAGTGCCATAACTCTGTTTGTTTCCAGTGAACAACTGAGGGGTAAAAATG
AAGCAGATAAAATGAAAGTATTTGATTATGTCTGTTTTGCTCAAGATGAA
TTACTTCCCAACGCTTGCAGATGGGTCTTCCCTATTTTAGATATATACCC
ATATAATAAACAAACTGTTGATTCAGCAAGAGATGGTTTGAAGAGAAGTC
TCTCTAAGCTTGATAAACATCTCTTAACTCGCACCTATTTGGTTGGTGAT
TACATCACTATTGCTGATATATGTAATGCATGTACTTTGTTACAAGTCTA
TCAACATGCTATGGACCCAACTTTCAGAAAACCATATGTCAATGTTAACA
GATGGTTTACTACTATTGTCAACCAACCAGAATTTAAGAAAATTGTAGGA
GAGGTCAAATTATGTGAGAAACAAGTTAATGAAGCTGAACTTGCTAGTAA
AAGTGGTGTCAAAGCTCAAGCACCAGAAGAGAAAAAAGAGAAGCCCAAAA
AAGAAAAGAAAGAACAACCAAAAAAAGAAAAGGAAGCAGAACCTGAGGAT
GCTGGAGATGCCATGGATGATGTATTGGCTGCTGAACCTAAATCAAAGGA
CCCATTTGATTCTATGCCAAAAGGCAGTTTTGTCATGGATGACTATAAAA
GATTTTACTCTAATAATGATGAGGCAAAATCTATTCCTTACTTCTGGGAA
AAATTCGACAAAGAAAACTATTCAATTTGGTTTGGAGAGTACAAGTACAA
TGATGAGCTTGCTAAAGTTTTTATGAGTTGTAATTTAATTACAGGAATGT
TTCAAAGACTGGACAAAATGAGAAAGCAAGCTTTTGCCTCATGCTGTCTG
TTTGGTTCAGATAATGATAGTAGTATTTCCGGAATTTGGGTGTGGAGAGG
ACATGATCTTGCCTTTACACTATGTCCAGACTGGCAAATTGATTATGAAT
CTTATGATTGGAAAAAATTAGATCCAGAAGCAAAAGAAACAAAAGATTTG
GTCACCCAATACTTTTCATGGACAGGCACTGATTCTAAGGGTCGTAAATT
TAATCAAGGAAAAATCTTTAAGTGA
Gene #29
Tef
Nucleotides 518-617 of SEQ ID NO: 26
Ef1gamma
SEQ ID NO: 27
CAACTTTCAGAAAACCATATGTCAATGTTAACAGATGGTTTACTACTATT
GTCAACCAACCAGAATTTAAGAAAATTGTAGGAGAGGTCAAATTATGTGA
Gene #30
eIF-2alpha
eukaryotic translation initiation factor 2 subunit
1-like isoform 1 (partial)
SEQ ID NO: 28
AACAAATTAATTAGAGTAGGAAAAACAGAACCTGTTGTTGTTATCAGAGT
TGACAAAGAAAAAGGTTATATTGATTTAAGTAAAAGAAGAGTATCACCAG
AAGATGTAGAAAAATGTACTGAAAGATATGCTAAGGCTAAAGCAGTTCAT
TCTATCTTGAGGCATGTTGCTGAAATCCTTCATTTTGATTCAGACAAACA
GTTGGAAGATCTTTATCAAAGAACTGCATGGAATTATGAAGATAAAACAA
AAAAGAAAGGTTCTTCATATGATTTCTTCAAACAAGCTGTCCTAGATCCC
AATACATTGATAGAATGTGGTCTTGATGAACATACAAGAGATGTCCTAGT
AAACAATATTCAACGTAAACTTACATCCCAAGCTGTAAAGATCAGAGCTG
ATATTGAAGTAGCATGTTATGGTTATGAAGGTATTGATGCTGTTAAGACA
GCCTTAAAAGCTGGTTTAGCAATGTCCACGGAGAAATTACCCATTAAAAT
CAATCTTATTGCTCCTCCATTGTATGTAATGACAACAGTAACACCAGAAA
AAGCTGATGGATTAAAAGCACTCCAAGAAGCAATCGACACCATTAAAATA
AAAATTGAAGAACTAGGTGGTGTGTTCCAAGTTCAAATGGCGCCCAAAGT
GGTTACAGCAAGTGACGAAGCTGAATTGGCTCGTCAAATG
Gene #30
Nucleotides 127-226 of SEQ ID NO: 28
eIF-2alpha
eukaryotic translation initiation factor 2 subunit
1-like isoform 1
SEQ ID NO: 29
TATGCTAAGGCTAAAGCAGTTCATTCTATCTTGAGGCATGTTGCTGAAAT
CCTTCATTTTGATTCAGACAAACAGTTGGAAGATCTTTATCAAAGAACTG
Gene #31
eIF3-S8
eukaryotic translation initiation factor 3
subunit C-like isoform 1 (partial)
SEQ ID NO: 30
CGAGACCTTATCTTGATGTCTCACCTTCAGGAAACTATTCAATACTCTGA
TCCTTCAACACAAATCTTGTACAATAGGACGATGGCTCACCTCGGTTTGT
GTGCATTCCGTCACGCGCACATCAAAGATGCTCATAATTGTCTGGTTGAT
TTAATGATGACTGGAAAAGTGAAGGAGTTGCTTGCCCAGGGTCTTATGCC
CCAACGACAACATGAGCGTAGCAAAGAACAGGAAAAAGTTGAAAAACAGC
GTCAGATTCCATTCCATATGCACATCAACCTAGAGCTGCTTGAGTGTGTT
TATTTGGTGTCAGCTATGCTCATAGAAATACCCTACATGGCTGCTCATGA
GTTCGATGCCCGCCGGAGGATGATTTCTAAAACTTTCTATCAACAACTTC
GTTCCAGTGAACGTCAAAGTCTGGTAGGACCCCCTGAATCGATGAGAGAG
CATGTAGTAGCCGCCAGTAAAGCTATGAGACAAGGAAATTGGAAAAATTG
TGTCAATTTTATAATAAATGAAAAAATGAACGCTAAAGTTTGGGATTTGT
TTTATGAGTCGAGTAAAACTCGTTCTATGCTGACTCGTCTTATCAAAGAA
GAATCTTTGAGAACTTATCTGTTCACATTCTCTCATGTGTATTCATCAAT
TTCTATGAATACCTTGTCGGCAATGTTTGAAATGGAAAAGCTTAGCGTAC
ATTCTATCATCTCTAAAATGATAATTAATGAAGAATTGATGGCATCTCTT
GATGATCCAACCCAAACAGTGGTCCTTCATCGATCTGAACCATCTAGACT
TCAAGCGCTAGCACTTCAATTGGCAGACAAAGTTAATAACTTCGTTGACT
CAAATGAACGTATCTTTGAAATGAAGCAAGGCAATTTCTTCCAAAGA
Gene #31
Nucleotides 761-860 of SEQ ID NO: 30
eIF3-S8
eukaryotic translation initiation factor 3
subunit C-like isoform 1
SEQ ID NO: 31
CCCAAACAGTGGTCCTTCATCGATCTGAACCATCTAGACTTCAAGCGCTA
GCACTTCAATTGGCAGACAAAGTTAATAACTTCGTTGACTCAAATGAACG
Gene #31
Nucleotides 761-860 of SEQ ID NO: 30 with T□C
substitution at nucleotide 793 of SEQ ID NO: 30,
to eliminate Xba I site
eIF3-S8
eukaryotic translation initiation factor 3
subunit C-like isoform 1
SEQ ID NO: 32
CCCAAACAGTGGTCCTTCATCGATCTGAACCACCTAGACTTCAAGCGCTA
GCACTTCAATTGGCAGACAAAGTTAATAACTTCGTTGACTCAAATGAACG
Gene #32
eIF5
eukaryotic translation initiation factor 5-like
isoform 1 (partial)
SEQ ID NO: 33
AATGTTACTGATGCATTTTATCGTTATAAAATGCCAAAGCTTATAGCTAA
GGTAGAGGGTAAAGGCAATGGAATTAAGACTGTCATTGTCAATATGGTAG
ATGTGGCAAAAGCACTGGGACGTCCTCCAACTTACCCCACTAAATATTTT
GGTTGTGAGTTGGGTGCACAAACAAAACTTGACCATAAAAATGATCGCTA
CATTGTTAATGGTTCCCATGATGTTACAAAGCTTCAGGACTTGCTTGATG
GATTCATCAGAAAATTTGTTCTTTGTCCTGAGTGTGACAATCCAGAGACA
GATCTAATTGTTTCAGCAAAGAAGCAAACCATTCAGCAAGGTTGCAAGGC
ATGTGGACATCATGGCCTGCTCACTTTCAACCACAAGTTGAATACTTTCA
TTTTAAAGAATCCTCCCAACTTGAATCCTGCTGTGCAAGGATCATCATTG
ACTGAGGGAAAGCGTCCTAAACGTGAAAGTAAGAAGCAAGATGCTAATGG
TGACATCTCTAAATCAGATGAGGAAGGTGACTGGCCAGTACAAGCTCCAG
AGAAGATTGGTGATAATGAGGATGATTGTGACTGGACTGAAGATGTGAGT
GAAGAAGCTGTAAGAGCTCGTATGCAAGATTTGACCACAGGAGTTAAAGG
TTTAACAATTACTGATGATTTAGATAAAACTGAAAAAGAACGGATGGATA
TATTTTATTCATGTGTAAAAGCAGCTCTTGAGAAAAATAATCTGGATGCT
AAGGAAATCCTGACTGAAGCTGAACGCTTGGAAGTGAAAACTAAAGCACC
CCTTGTTCTAGCTGAACTGCTTTTTGATGATAAAATTCACATTCAGATGA
AAAAACACCGCATTTTATTGTTGCGTTTCACCCATGAAGATACTAAAGCC
CAACGTTATCTCTTAAATGGAATAGAACAAGTCATAGCTTTGCATAAAGA
TGTACTTTTAGCAAAAGTACCAGCTATACTGAAACTTTTCTATGATGCTG
ATATTTTGGAGGAAAAAGTATTGCTAGAATGGGCTGAAAAGGTTTCCAAA
AAATATGTCTCTAAAGAGCTGAGTGCAGAGATTCGTTCTCGTGCTGAACC
ATTTATTAAATGGTTACGT
Gene #32
Nucleotides 704-803 of SEQ ID NO: 33
eIF5
eukaryotic translation initiation factor 5-like
isoform 1
SEQ ID NO: 34
TTTATTCATGTGTAAAAGCAGCTCTTGAGAAAAATAATCTGGATGCTAAG
GAAATCCTGACTGAAGCTGAACGCTTGGAAGTGAAAACTAAAGCACCCCT
Gene #34
hay
DNA excision repair protein haywire-like (partial)
SEQ ID NO: 35
TTTCTTATAGCTATAGCAGAACCTGTGTGTCGTCCTTTTACACGATTCAC
TGAATACAAGTTAACAGCATATTCTTTATATGCTGCGGTCAGTGTCGGAT
TACAGACTCATGATATTATTGAATATCTTAAAAGACTGAGTAAAACATCC
GTGCCTGATGGTATAGTAGAGTTTATCACACTTTGTACATTATCTTATGG
AAAGGTTAAATTAGTGCTAAAACACAATAGATATTTCATAGAATCACAAT
TTGCAGATGTTTTACAAAAACTTTTAAAAGATCCTGTGATACAAGAATGT
CGTCTAAGACGTGATGTTGAAGATTCACAAACTCTTATCAGTGAAACTGA
TAAG
Gene #34
Nucleotides 286-354 of SEQ ID NO: 35
hay
DNA excision repair protein haywire-like
SEQ ID NO: 36
GTGATACAAGAATGTCGTCTAAGACGTGATGTTGAAGATTCACAAACTCT
TATCAGTGAAACTGATAAG
Gene #35
Hel25E
ATP-dependent RNA helicase WM6-like (partial)
SEQ ID NO: 37
GTGCAAGAAATATTCCGAAATACTCCTCATCAAAAACAAGTTATGATGTT
TTCAGCCACTTTGAGCAAAGAAATCCGTCCAGTGTGCAAAAAGTTTATGC
ATAGATCCAATGGAGGTTTATGTGGATGATGATGCCAAACTTACTCTGCA
TGGACTTCAACAACATTACGTCAAACTGAAGGAGAATGAAAAAAATAAAA
ACTTTTTGAACTACTTGATGCCCTAGATTTCAATCAAGTTGTGGTATTTG
TAAAGTCGGTCACTCGTTGTATTGCTCTATCATCGCTCCTATCGGAACAG
AATTTTCCTGCTACTGGCATCCACCGTGGTATGACTCAAGAAGAAAGACT
TAAAAAATACCAAGAATTCAAAGATTTCCAAAAGAGAATCCTTGTGGCCA
CCAACTTATTTGGTCGTGGTATGGACATTGAGAAGGTTAACATTGTATTC
AACTATGACATGCCTGAAGATTCTGACACTTATCTACACAGAGTGGCACG
TGCAGGGCGATTCGGCACAAAGGGTTTAGCCATCACCTTTGTTTGTGATG
AAAATGATGCTAAAATTTTAAACAACGTACAAGAGAGATTTGATGTGAGC
ATTACTGTACTACCTGATGAAATTGACTTGTCGACCTATATTGAAGGACG
ATAA
Gene #35
Nucleotides 462-561 of SEQ ID NO: 37
Hel25E
ATP-dependent RNA helicase WM6-like
SEQ ID NO: 38
GCCTGAAGATTCTGACACTTATCTACACAGAGTGGCACGTGCAGGGCGAT
TCGGCACAAAGGGTTTAGCCATCACCTTTGTTTGTGATGAAAATGATGCT
Gene #37
Hr38
ecdysone receptor isoform B1 (partial)
SEQ ID NO: 39
ATTGTAGAATTTGCTAAGAGGTTACCTGGTTTCGACAAATTAGTTAGGGA
AGATCAAATTTCATTACTTAAGGCTTGTTCGAGTGAAGTAATGATGTTAC
GAATGGCAAGGAGGTATGATGCTCCTTCTGATTCGATATTGTTTGCAAAT
AACCAACCATATACTAGGGAGGCATACAAGTCTGCCGATATGGGAGAAAC
AGTAGATGATCTGCTCAAATTTTGTAGGCTTATGTATTCAATGAAAGTTG
ACAATGCAGAATATGCGTTGCTGACAGCCATTGTTATATTT
Gene #37
Nucleotides 113-212 of SEQ ID NO: 39
Hr38
ecdysone receptor isoform B1
SEQ ID NO: 40
GGTATGATGCTCCTTCTGATTCGATATTGTTTGCAAATAACCAACCATAT
ACTAGGGAGGCATACAAGTCTGCCGATATGGGAGAAACAGTAGATGATCT
Gene #40
mask
hypothetical protein (partial)
SEQ ID NO: 41
GAATTGCTGCTCAAGCGAGGTGCCAATAAAGAACATAGAAATGTATCGGA
TTATACTCCGCTAAGCCTAGCTGCAAGTGGAGGATATGTGAATATAATCA
AACTGCTGCTTACACATGGCGCTGAGATAAACTCCCGCACTGGATCTAAG
CTAGGAATATCACCTCTTATGTTAGCAGCTATGAATGGCCACACTCCAGC
TGTAAAACTCTTGCTGGATATGGGAAGTGATATTAATGCTCAGATTGAAA
CCAATAGGAATACAGCACTTACTCTAGCATGCTTCCAAGGAAGGCATGAA
GTTGTGAGTTTGCTGTTGGACAGGAAAGCTAACGTAGAGCATAGAGCAAA
GACTGGACTTACGCCCTTGATGGAAGCTGCAAGTGGGGGCTACACCGATG
TTGGGCGCGTTCTATTAGATAAAGGTGCTGATGTAAATGCCCCTCCTGTG
CCTTCATCTCGAGATACTGCATTAACTATTGCTGCTGATAAAGGTCATGG
CAGATTCGTAGACCTTTTATTGTCCAGAGGAGCCCAAGTAGAAGTTAAAA
ATAAAAAAGGAAACTCTCCCCTATGGTTGGCTGCCAATGGTGGCCATCAG
AGTGTTGTGGCACTACTTTGGAAACATCGTGCAGATATTGATTCTCAAGA
CAACCGTCAAGTTTCATGTTTGATGGCTGCATTCCGTAAAGGTCACTGCA
AAGTGGTTCAGTGGATGGTTAATCATGTTGCTCAATTTCCTAGTGATCAG
GAAATGACGCGATATATACAAACTGTCAATGATAAGGACCTTCTAAATAA
ATGTCAAGAATGTTTGATGTCAATTAGAGCTGCAAAAAATCAACAAGCTG
AGAAAGCTAACAAAAATGCTAATATACTTTTAGAAGAACTAGATATGGAA
AAGTGGCGGGAAGAA
Gene #40
Nucleotides 121-220 of SEQ ID NO: 40
mask
hypothetical protein
SEQ ID NO: 42
GCTGAGATAAACTCCCGCACTGGATCTAAGCTAGGAATATCACCTCTTAT
GTTAGCAGCTATGAATGGCCACACTCCAGCTGTAAAACTCTTGCTGGATA
Gene #41
mor
SWI/SNF complex subunit SMARCC2-like (partial)
SEQ ID NO: 43
AGATTGAATCCGATGGAATATGTCACTAGTACAGCTTGTAGGCGAAATTT
AGCAGGGGATGTGTGTGCTATAATGCGAGTTCATGCTTTCTTAGAACAGT
GGGGATTAATTAATTACCAGGTTGATAGCGATTCAAAACCATCTGCTATT
GGTCCACCACCTACATCTCACTTCCATGTTTTAACTGACACTCCATCTGG
ACTTCAACCTGTTAATCCTCCTAAAACAACACAACCCTCAGCTGCTAAGA
TATTGTTGGATATGGATAAGAAACCAGATACGCTACTCAAGAAAGAAGGC
TCTGAGATCCCATCTAATTTTGGATTGAAATTAGACCAGTATGCTAAGAA
GCCAGCAGTTTTGAGAAACAAACAAGCTGCTAGCATGGTTCGAGATTGGA
CAGAACAAGAAACTTTGCTCTTGCTGGAAGCTCTAGAAATGTACAAAGAT
GATTGGAATAAAGTTTGTGAACATGTTGGAAGTCGAACTCAGGATGAGTG
CATTTTACATTTCTTAAGATTGCCAATTGAAGACCCATATTTAGAAGATC
CTGAGTCTGGTGGAGGTGCATTAGGTCCTTTAGCTTATCAACCAATACCA
TTCAGCAAGGCTGGTAATCCCATCATGTCAACTGTAGCCTTTTTAGCATC
AGTTGTTGATCCCCGAGTTGCTTCTTCTGCTGCAAAAGCTGCCATGGAAG
AATTTGCACGTATTAAGGATGAAGTCCCAGCTGCTATTATGGATGCACAC
ATCAAGAATGTTGAAGCCTCCACCGCAGACGGAAAATATGATCCTGCTGC
AGGACTTTTGCAGAGTGGAATAGCAGGAACTGTT
Gene #41
Nucleotides 248-347 of SEQ ID NO: 43
mor
SWI/SNF complex subunit SMARCC2-like
SEQ ID NO: 44
AGATATTGTTGGATATGGATAAGAAACCAGATACGCTACTCAAGAAAGAA
GGCTCTGAGATCCCATCTAATTTTGGATTGAAATTAGACCAGTATGCTAA
Gene #47
RpS2
40S ribosomal protein S2-like (partial)
SEQ ID NO: 45
AAGGAAGCTGAAAAGGAATGGGCACCTGTCACCAAATTGGGTCGCTTGGT
TAGAGATGGTAAAATTCAATCATTAGAACAAATTTACTTGTTCTCTCTAC
CCATCAAGGAATTTGAAATCATTGACTTCTTTATTGGATCAGTCTTAAAA
GATGAGGTACTCAAAATCATGCCTGTACAGAAACAAACCAGAGCTGGTCA
AAGAACTAGATTCAAGGCTTTTGTTGCCATTGGTGACTCTAATGGACATA
TTGGTTTAGGTGTTAAGTGCTCTAAGGAAGTAGCAACTGCCATCCGTGGA
GCTATCATCTTAGCTAAATTGTCTGTTGTTCCAGTCAGAAGAGGTTACTG
GGGAAACAAGATTGGTAAACCCCACACCGTACCTTGCAAGGTTACTGGTA
AATGTGGTTCAGTTCAAGTACGTCTCATCCCTGCTCCTCGTGGTACAGGT
ATTGTAGGAGCTCCAGTCCCTAAGAAATTATTGCAGATGGCTGGTATTGA
AGATTGTTATACCTCAGCTAGAGGTTCAACTTGTACTCTTGGTAACTTCG
CCAAAGCTACATATGCTGCTATTGCCAAGACATATGCTTACCTGACACCA
GATTTATGGAAAGACAACCCACTTAGAAAAGCCCCTTACAGTGAATTCAG
TGAGTTCTTGGAAAAGAATCATCGCATT
Gene #47
Nucleotides 579-678 of SEQ ID NO: 45
RpS2
40S ribosomal protein S2-like
SEQ ID NO: 46
GACATATGCTTACCTGACACCAGATTTATGGAAAGACAACCCACTTAGAA
AAGCCCCTTACAGTGAATTCAGTGAGTTCTTGGAAAAGAATCATCGCATT
Gene #48
RpS5a
40S ribosomal protein S5-like isoform 1 (full)
SEQ ID NO: 47
ATGGCCGAAGATTGGGATACTGATCCAGCTTATCCTGAAATAGCCACTGG
CCCAGTAGGATTATCTTCAATTGCTGCTCCTGCTGAATTACCAGAAATTA
AATTATTTGGAAGATGGAGTTGTGATGATGTCCAAGTTAGTGATATGTCC
CTCCAGGATTATATTGCTGTTAAAGAAAAGAATGCAAAATATTTACCTCA
CTCAGCTGGAAGATATGCTGCCAAAAGATTCCGTAAAGCACAATGTCCCA
TTGTAGAAAGATTAACTAACTCACTTATGATGCACGGACGTAACAATGCT
AAAAAATTAATGGCTGTTAGAATTGTTAAACACGCTTTTGAAATTATTCA
TTTGTTAACTGGAGAGAATCCCCTCCAAGTCTTGGTAACTGCCATCATCA
ATTCAGGACCAAGAGAAGATTCAACACGTATTGGACGTGCCGGTACAGTA
AGAAGACAAGCTGTTGATGTATCACCTTTAAGAAGAGTAAATCAAGCTAT
CTGGTTATTGTGTACTGGTGCCAGAGAAGCTGCCTTCAGAAATATTAAAA
CTATTGCTGAATGTGTTGCTGATGAACTTATCAATGCTGCTAAGGGATCA
TCTAACTCCTATGCTATTAAGAAGAAAGATGAATTGGAACGTGTCGCCAA
GTCCAATCGTTAA
Gene #48
Nucleotides 1-100 of SEQ ID NO: 47
RpS5a
40S ribosomal protein S5-like isoform 1
SEQ ID NO: 48
ATGGCCGAAGATTGGGATACTGATCCAGCTTATCCTGAAATAGCCACTGG
CCCAGTAGGATTATCTTCAATTGCTGCTCCTGCTGAATTACCAGAAATTA
Gene #53
Trip1
eukaryotic translation initiation factor 3
subunit 2 beta-like (full)
SEQ ID NO: 49
ATGAAACCATTAATTCTTCAAGGTCATGAAAGATCAATCACCCAAATTAA
ATACAACAGAGAAGGTGATATCTTAATCAGTTGTGCCAAGGATGCTGATG
CAAATGTATGGTATTCAGTTAATGGTGAGAGAGCCGGAACTCTTAGTGGT
AGTAAAGGTACCATTTGGACAATTGATATTGACTGGATGACTACAAGAGT
ACTGACTGGTCATGCTGATGGAAAACTTAAAATGTGGGATATTTCAAATG
GAACAACAATCAGTGATATTCCAACATTCTCAAATTGTGCTGTCAGAACA
TGTGGGTTTAGTTACTCATCAAATTTATGTGCTTACAGTAATGATGACAC
CATGGGAAATAAATGTTATCTCTCCGTTCTTGATGTTAGGACTACTGATG
CTACAAATTCAGGAGACCCAGTTGTTAAGATGCAAGTGACAGATGAATCT
GCCAAAATTACATCTATGTTGTGGGGTAACTTAGATGAATACATCATCAC
AGGCCATGCCAAAGGTGATATTTGCACCTGGGATATAAGAATGGGAAGAC
ATTTAGAAAGTGTTAACGCTCATCCTGGACAACCAATTAATGATATGCAA
TTTTCCAAGGACTCTACAATGTTTATTACAGCCTCCAAAGATCATACTGC
TAAATTATTTAGTTCTGGAGATTGTCAACTTTTGAAAACATACACCACTG
AAAGACCTGTCAACAGTGCTGCTTTATCACCAATCCTACCTCATGTTGTA
CTTGGAGGAGGTCAAGAAGCTCGTGAAGTAACAACAACTTCGACTAAAGT
CGGAAAATTCGATGCTAGATTTTATCATTTAATATTTGAAGAAGAATTTG
CACGTATTAAAGGTCATTTTGGTCCTATTAACTCTTTAGCTTTTCATCCA
GATGGAAAATCATATGCTAGTGGTGGAGAAGACGGTTTTGTTAGGTTACA
TACATTTGATCAATCTTATTTCGATTATACTTTTGACATTTAG
Gene #53
Nucleotides 333-432 of SEQ ID NO: 49
Trip1
eukaryotic translation initiation factor 3
subunit 2 beta-like
SEQ ID NO: 50
TTACAGTAATGATGACACCATGGGAAATAAATGTTATCTCTCCGTTCTTG
ATGTTAGGACTACTGATGCTACAAATTCAGGAGACCCAGTTGTTAAGATG
Gene #54
tws
protein phosphatase PP2A 55 kDa regulatory
subunit-like isoform 3 (partial)
SEQ ID NO: 51
TCTGGACGGTATATGATCTCAAGAGATTACCTTTGTGTTAAAGTTTGGGA
TTTACATATGGAGTCTAGACCAGTAGAAACTTACCCAGTTCATGAATACC
TTAGATCAAAATTATGTTCACTATATGAAAATGACTGTATATTTGACAAG
TTTGAATGTTGTTGGTCCGGTAACGATTCAGCAATTATGACAGGCTCTTA
TAATAATTTCTTCCGTATGTTTGATAGAATTAATAAACGAGATGCCACAC
TAGAGGCATCAAGGGAAATAGCAAAGCCTAAAACACTACTTAGACCTAGA
AAAGTATGTACAGCTGGTAAAAGGAAAAAAGATGAAATCAGTGTAGACTG
TTTGGACTTCAATAAGAAAATCCTTCATACGGCCTGGCATCCTAGTGAAA
ATATAATTGCTGTAGCGGCAACTAACAATTTATATTTGTTCCATGATAAG
TTGTAG
Gene #54
Nucleotides 209-308 of SEQ ID NO: 51
tws
protein phosphatase PP2A 55 kDa regulatory
subunit-like isoform 3
SEQ ID NO: 52
TCTTCCGTATGTTTGATAGAATTAATAAACGAGATGCCACACTAGAGGCA
TCAAGGGAAATAGCAAAGCCTAAAACACTACTTAGACCTAGAAAAGTATG
Gene #55
Ubc-E2H
ubiquitin-conjugating enzyme E2 H-like (partial)
SEQ ID NO: 53
ATGTCTTCACCAAGTGCGGGAAAGCGACGGATGGATACGGATGTCATAAA
ACTAATTGAAAGTAAACATGAAGTCACCATTTTGGGAGGACTAAATGAAT
TCTGTGTTAAATTTTTCGGACCCAGAGATACGCCATATGAAGGAGGAGTT
TGGAAAGTTAGAGTACATCTTCCAGAACACTACCCTTTCAAATCACCCTC
TATTGGGTTCATGAATAAAGTATATCACCCTAATATAGATGAAGTCTCAG
GTACGGTGTGTCTCGACGTCATTAACCAAGCGTGGACGGCGCTATATGAT
CTTTCAAATATTTTTGTATCTTTCTTACCTCAACTGTTAACCTACCCCAA
CCCT
Gene #55
Nucleotides 98-192 of SEQ ID NO: 53
Ubc-E2H
ubiquitin-conjugating enzyme E2 H-like
SEQ ID NO: 54
AATTCTGTGTTAAATTTTTCGGACCCAGAGATACGCCATATGAAGGAGGA
GTTTGGAAAGTTAGAGTACATCTTCCAGAACACTACCCTTTCAAA
Gene #56
Uev1A
ubiquitin-conjugating enzyme-like (full)
SEQ ID NO: 55
ATGGCAGGAGTGGTTGTGCCTAGAAATTTTCGTTTACTTGAAGAATTAGA
GCAAGGTCAACGAGGTGTTGGAGATGGAACAATCAGTTGGGGTCTTGAAA
ATGATGATGATATGACTTTGACACATTGGACTGGAATGATTATAGGACCA
CCTAGGACACCATATGAAAATAGAATGTACAGTTTAAGGATTGAATGTGG
ACCGAGATATCCCGACGAACCACCAAGTGCCCGATTCATATCAAGAATCA
ACATGAACTGTATAAATAGTAATTCTGGAATTGTGGATCAAAAAAATGTA
CCAGTTCTAGCTAGATGGCAACGAGAGTATACTATCAAGTCTTTATTACA
AGAACTGCGAAGATTAATGACTGTAAAAGATAATACTAAACTCTCACAAC
CACCTGAAGGGAGCACATTTTAA
Gene #56
Nucleotides 324-423 of SEQ ID NO: 55
Uev1A
ubiquitin-conjugating enzyme-like
SEQ ID NO: 56
AGAGTATACTATCAAGTCTTTATTACAAGAACTGCGAAGATTAATGACTG
TAAAAGATAATACTAAACTCTCACAACCACCTGAAGGGAGCACATTTTAA
P1- CaMV 35S Promoter & Omega UTR
SEQ ID NO: 57
AGATTAGCCTTTTCAATTTCAGAAAGAATGCTAACCCACAGATGGTTAGA
GAGGCTTACGCAGCAGGTCTCATCAAGACGATCTACCCGAGCAATAATCT
CCAGGAAATCAAATACCTTCCCAAGAAGGTTAAAGATGCAGTCAAAAGAT
TCAGGACTAACTGCATCAAGAACACAGAGAAAGATATATTTCTCAAGATC
AGAAGTACTATTCCAGTATGGACGATTCAAGGCTTGCTTCACAAACCAAG
GCAAGTAATAGAGATTGGAGTCTCTAAAAAGGTAGTTCCCACTGAATCAA
AGGCCATGGAGTCAAAGATTCAAATAGAGGACCTAACAGAACTCGCCGTA
AAGACTGGCGAACAGTTCATACAGAGTCTCTTACGACTCAATGACAAGAA
GAAAATCTTCGTCAACATGGTGGAGCACGACACACTTGTCTACTCCAAAA
ATATCAAAGATACAGTCTCAGAAGACCAAAGGGCAATTGAGACTTTTCAA
CAAAGGGTAATATCCGGAAACCTCCTCGGATTCCATTGCCCAGCTATCTG
TCACTTTATTGTGAAGATAGTGGAAAAGGAAGGTGGCTCCTACAAATGCC
ATCATTGCGATAAAGGAAAGGCCATCGTTGAAGATGCCTCTGCCGACAGT
GGTCCCAAAGATGGACCCCCACCCACGAGGAGCATCGTGGAAAAAGAAGA
CGTTCCAACCACGTCTTCAAAGCAAGTGGATTGATGTGATATCTCCACTG
ACGTAAGGGATGACGCACAATCCCACTATCCTTCGCAAGACCCTTCCTCT
ATATAAGGAAGTTCATTTCATTTGGAGAGAACACGGGGGACTCTAGATAT
TTTTACAACAATTACCAACAACAACAAACAACAAACAACATTACAATTAC
TATTTACAATTACA
sgFIMV Promoter
SEQ ID NO: 58
TTTACAGTAAGAACTGATAACAAAAATTTTACTTATTTCCTTAGAATTAA
TCTTAAAGGTGATAGTAAACAAGGACGATTAGTCCGTTGGCAAAATTGGT
TCAGCAAGTATCAATTTGATGTCGAACATCTTGAAGGTGTAAAAAACGTT
TTAGCAGATTGCCTCACGAGAGATTTTAATGCTTAAAAACGTAAGCGCTG
ACGTATGATTTCAAAAAACGCAGCTATAAAAGAAGCCCTCCAGCTTCAAA
GTTTTCATCAACACAAATTCTAAAAACAAAATTTTTTAGAGAGGGGGAGT
G
AtActin7 Terminator including 3UTR
SEQ ID NO: 59
GTGTGTCTTGTCTTATCTGGTTCGTGGTGGTGAGTTTGTTACAAAAAAAT
CTATTTTCCCTAGTTGAGATGGGAATTGAACTATCTGTTGTTATGTGGAT
TTTATTTTCTTTTTTCTCTTTAGAACCTTATGGTTGTGTCAAGAAGTCTT
GTGTACTTTAGTTTTATATCTCTGTTTTATCTCTTCTATTTTCTTTAGGA
TGCTTGTGATGATGCTGTTTTTTTTTGTCCCTAAGCAAAAAAATATCATA
TTATATTTGGTCCTTGGTTCATTTTTTTGGTTTTTTTTTGTCTTCACATA
TAAATATTGTTTGAATGTCTTCAATCTTTTATTTGTATGAGACAATTATT
TAAGTATCGGGTGACAATGCAGCTATTATGTATTGTCGATTGTTATATTG
GCGCCCAAAATATATACTTAGCCTAAGAATTTGGTAAGTGAGTGGCTTAT
GTTTTACTCCAGCAAAAATTGTGTGTGTATTACCATTCTGATGCGAAACA
AGAAAAGAATTTGATCTAAGAAACCAAGTTTATTCACTAGTTAAAAAACA
AATGACCTAATGTAATCGACTCCACATATCAAAATACGTAAAACAAACAT
TGTATGTTGACAAAAGGGAAAAGAAATGATTTATTTGGTTAAAAAGAAAG
CTGGATTCAATTGCAACAGTTTAGTCGAAATCATTTTGAAAGGCTTACAA
TGGATTGAATGTGAATATTCCATTAAGCCGCTTCTGTCTACACAGAATGT
TACGCTTGGAGAGCAGCAATCATTTTCACGTTTTTATCTTTTTAGGTGGA
CATGTATATTATTGGTTACGCCTTTGGAGTTTTTCGAAATTTATTTCTTT
CAAATCACAAGATGACTAAACATCACAATCTGTTTATCTTCCTAACTAGT
TAAATTTTTGTCCCCACCATT
NOS Terminator
SEQ ID NO: 60
GATCGTTCAAACATTTGGCAATAAAGTTTCTTAAGATTGAATCCTGTTGC
CGGTCTTGCGATGATTATCATATAATTTCTGTTGAATTACGTTAAGCATG
TAATAATTAACATGTAATGCATGACGTTATTTATGAGATGGGTTTTTATG
ATTAGAGTCCCGCAATTATACATTTAATACGCGATAGAAAACAAAATATA
GCGCGCAAACTAGGATAAATTATCGCGCGCGGTGTCATCTATGTTACTAG
ATC
Loop Sequence
SEQ ID NO: 61
GGCTCGAACGAGCCGACTAATTGTCTTTAAACGCGCGATATAAGCGCACA
ATGCTCGAGAAACGATAAACTCTATCGCTCTGTCGCGTGCGTGGCATCTT
CGCGCG
Construct 1, hpRNA
SEQ ID NO: 62
AGCTTTAGACCGTCTGGTTACCAAAAAAGCTGGTTTCTCTACTTCTCACA
TCATCTGTGGCCAAACATACCCTAGAAAAGTTGACGTCATCGTAACGGGA
CTCAATTACATCTGGAAAATTGGAGCGTACTCTCAATGTTCACGAGAAAT
TAGTAATTGGATTGACTCATCACCCTCATCAAAATCTCCTAGGAACCTAC
CAACTTTCAGAAAACCATATGTCAATGTTAACAGATGGTTTACTACTATT
GTCAACCAACCAGAATTTAAGAAAATTGTAGGAGAGGTCAAATTATGTGA
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCTCACATAATTTGACCTCTCCTACAATTTTCTTAAATTCTGGTTG
GTTGACAATAGTAGTAAACCATCTGTTAACATTGACATATGGTTTTCTGA
AAGTTGGTAGGTTCCTAGGAGATTTTGATGAGGGTGATGAGTCAATCCAA
TTACTAATTTCTCGTGAACATTGAGAGTACGCTCCAATTTTCCAGATGTA
ATTGAGTCCCGTTACGATGACGTCAACTTTTCTAGGGTATGTTTGGCCAC
AGATGATGTGAGAAGTAGAGAAACCAGCTTTTTTGGTAACCAGACGGTCT
AAAGCT
Construct 1, sense mRNA
SEQ ID NO: 63
AGCTTTAGACCGTCTGGTTACCAAAAAAGCTGGTTTCTCTACTTCTCACA
TCATCTGTGGCCAAACATACCCTAGAAAAGTTGACGTCATCGTAACGGGA
CTCAATTACATCTGGAAAATTGGAGCGTACTCTCAATGTTCACGAGAAAT
TAGTAATTGGATTGACTCATCACCCTCATCAAAATCTCCTAGGAACCTAC
CAACTTTCAGAAAACCATATGTCAATGTTAACAGATGGTTTACTACTATT
GTCAACCAACCAGAATTTAAGAAAATTGTAGGAGAGGTCAAATTATGTGA
Construct 2, hpRNA
SEQ ID NO: 64
CCCAAACAGTGGTCCTTCATCGATCTGAACCACCTAGACTTCAAGCGCTA
GCACTTCAATTGGCAGACAAAGTTAATAACTTCGTTGACTCAAATGAACG
GCCTGAAGATTCTGACACTTATCTACACAGAGTGGCACGTGCAGGGCGAT
TCGGCACAAAGGGTTTAGCCATCACCTTTGTTTGTGATGAAAATGATGCT
AGAGTATACTATCAAGTCTTTATTACAAGAACTGCGAAGATTAATGACTG
TAAAAGATAATACTAAACTCTCACAACCACCTGAAGGGAGCACATTTTAA
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCTTAAAATGTGCTCCCTTCAGGTGGTTGTGAGAGTTTAGTATTAT
CTTTTACAGTCATTAATCTTCGCAGTTCTTGTAATAAAGACTTGATAGTA
TACTCTAGCATCATTTTCATCACAAACAAAGGTGATGGCTAAACCCTTTG
TGCCGAATCGCCCTGCACGTGCCACTCTGTGTAGATAAGTGTCAGAATCT
TCAGGCCGTTCATTTGAGTCAACGAAGTTATTAACTTTGTCTGCCAATTG
AAGTGCTAGCGCTTGAAGTCTAGGTGGTTCAGATCGATGAAGGACCACTG
TTTGGG
Construct 2, sense mRNA
SEQ ID NO: 65
CCCAAACAGTGGTCCTTCATCGATCTGAACCACCTAGACTTCAAGCGCTA
GCACTTCAATTGGCAGACAAAGTTAATAACTTCGTTGACTCAAATGAACG
GCCTGAAGATTCTGACACTTATCTACACAGAGTGGCACGTGCAGGGCGAT
TCGGCACAAAGGGTTTAGCCATCACCTTTGTTTGTGATGAAAATGATGCT
AGAGTATACTATCAAGTCTTTATTACAAGAACTGCGAAGATTAATGACTG
TAAAAGATAATACTAAACTCTCACAACCACCTGAAGGGAGCACATTTTAA
Construct 3, hpRNA
SEQ ID NO: 66
AGATATTGTTGGATATGGATAAGAAACCAGATACGCTACTCAAGAAAGAA
GGCTCTGAGATCCCATCTAATTTTGGATTGAAATTAGACCAGTATGCTAA
TTACAGTAATGATGACACCATGGGAAATAAATGTTATCTCTCCGTTCTTG
ATGTTAGGACTACTGATGCTACAAATTCAGGAGACCCAGTTGTTAAGATG
TCTTCCGTATGTTTGATAGAATTAATAAACGAGATGCCACACTAGAGGCA
TCAAGGGAAATAGCAAAGCCTAAAACACTACTTAGACCTAGAAAAGTATG
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCCATACTTTTCTAGGTCTAAGTAGTGTTTTAGGCTTTGCTATTTC
CCTTGATGCCTCTAGTGTGGCATCTCGTTTATTAATTCTATCAAACATAC
GGAAGACATCTTAACAACTGGGTCTCCTGAATTTGTAGCATCAGTAGTCC
TAACATCAAGAACGGAGAGATAACATTTATTTCCCATGGTGTCATCATTA
CTGTAATTAGCATACTGGTCTAATTTCAATCCAAAATTAGATGGGATCTC
AGAGCCTTCTTTCTTGAGTAGCGTATCTGGTTTCTTATCCATATCCAACA
ATATCT
Construct 3, sense mRNA
SEQ ID NO: 67
AGATATTGTTGGATATGGATAAGAAACCAGATACGCTACTCAAGAAAGAA
GGCTCTGAGATCCCATCTAATTTTGGATTGAAATTAGACCAGTATGCTAA
TTACAGTAATGATGACACCATGGGAAATAAATGTTATCTCTCCGTTCTTG
ATGTTAGGACTACTGATGCTACAAATTCAGGAGACCCAGTTGTTAAGATG
TCTTCCGTATGTTTGATAGAATTAATAAACGAGATGCCACACTAGAGGCA
TCAAGGGAAATAGCAAAGCCTAAAACACTACTTAGACCTAGAAAAGTATG
Construct #4, hpRNA
SEQ ID NO: 68
TCTTCCGTATGTTTGATAGAATTAATAAACGAGATGCCACACTAGAGGCA
TCAAGGGAAATAGCAAAGCCTAAAACACTACTTAGACCTAGAAAAGTATG
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCCATACTTTTCTAGGTCTAAGTAGTGTTTTAGGCTTTGCTATTTC
CCTTGATGCCTCTAGTGTGGCATCTCGTTTATTAATTCTATCAAACATAC
GGAAGA
Construct #5, hpRNA
SEQ ID NO: 69
TTACAGTAATGATGACACCATGGGAAATAAATGTTATCTCTCCGTTCTTG
ATGTTAGGACTACTGATGCTACAAATTCAGGAGACCCAGTTGTTAAGATG
TCTTCCGTATGTTTGATAGAATTAATAAACGAGATGCCACACTAGAGGCA
TCAAGGGAAATAGCAAAGCCTAAAACACTACTTAGACCTAGAAAAGTATG
GCGCGCGAAACAACGGTAATCAACCGGCAATTATTAATCGTACATGCGCG
GCGCACTCGAGTGCATTATCCCTCGTCATCACCAAAGCGCCACATTATGC
TTCTTCCATACTTTTCTAGGTCTAAGTAGTGTTTTAGGCTTTGCTATTTC
CCTTGATGCCTCTAGTGTGGCATCTCGTTTATTAATTCTATCAAACATAC
GGAAGACATCTTAACAACTGGGTCTCCTGAATTTGTAGCATCAGTAGTCC
TAACATCAAGAACGGAGAGATAACATTTATTTCCCATGGTGTCATCATTA
CTGTAA
Construct #5, sense mRNA
SEQ ID NO: 70
TTACAGTAATGATGACACCATGGGAAATAAATGTTATCTCTCCGTTCTTG
ATGTTAGGACTACTGATGCTACAAATTCAGGAGACCCAGTTGTTAAGATG
TCTTCCGTATGTTTGATAGAATTAATAAACGAGATGCCACACTAGAGGCA
TCAAGGGAAATAGCAAAGCCTAAAACACTACTTAGACCTAGAAAAGTATG
Gene #57
Vps23
NADH-ubiquinone oxidoreductase, 20 Kd subunit
(full)
SEQ ID NO: 71
ATGCTGGCACTTCGCCCTGCAGTTTTGGCAAGAGCCTCCAATGTATGTGT
AAGAAGTCTTTCTTCTCCTCCATCTACAAGTACCAAATCAGGATCTTCTG
TTGAAGATGTCACTAAAAACTCTGCTATTGAAGCTGAAACAAGAGCTCCT
GTTAGAAGAGAGGATTACAGTCCTTTCAATGTTACAAGAAAAGATAACAT
GTTTGAGTACACTCTAGCAAGACTTGATGATGTTCTCAACTGGGGAAGAA
AAAATTCTATTTGGCCTCTAACTTTCGGTCTAGCATGTTGTGCTGTAGAA
ATGATGCACATTGCTGCTCCTAGATATGATATGGACAGGTATGGTGTAGT
CTTCCGTGCCTCTCCTAGACAAGCTGATGTTATTATTGTAGCTGGAACCT
TAACCAATAAAATGGCGCCTGCTCTTAGAAAAGTTTATGACCAGATGTTG
GATCCACGTTGGGTAATTTCAATGGGTAGTTGTGCAAATGGTGGTGGATA
TTACCATTATTCTTATTCAGTTGTTCGAGGATGTGATCGTATTATTCCTG
TAGATATTTATGTACCAGGATGTCCACCAACTGCTGAGGCACTTATGTAT
GGTATCTTACAATTACAAAAGAAAGTTAAGAGAATGAGAACTTTCCAGAT
GTGGTACAGACGATAA
Gene #57
Nucleotides 167-266 of SEQ ID NO: 71
Vps23
NADH-ubiquinone oxidoreductase, 20 Kd subunit
SEQ ID NO: 72
ACAGTCCTTTCAATGTTACAAGAAAAGATAACATGTTTGAGTACACTCTA
GCAAGACTTGATGATGTTCTCAACTGGGGAAGAAAAAATTCTATTTGGCC
Gene #58
Vps28
Vacuolar protein sorting 28 (partial)
SEQ ID NO: 73
GATCGTCCTATTACAATCAAAGATGATAAAGGAAATACATCCAAATGTAT
TGCTGATATTGTTTCATTATTTATTACCATCATGGACAAATTACGTCTAG
AAATAAAAGCTATGGATGAGTTACATCCTGATCTACGTGATCTCATGGAT
ACTATGAACAGATTGAGTATTTTGCCCTCCAACTTTGAAGGAAAGGAAAA
GGTTTCTAATTGGTTGAATGITTTGACAGCTATGTCAGCCAGTGATGAGC
TCAATGAAACACAAGTGAGACAGTTGCTATTTGATTTAGAAACATCTTAC
AATGCATTCAATAAGATCCTTCATCAATCTGCTTGA
Gene #58
Nucleotides 167-266 of SEQ ID NO: 73
Vps28
Vacuolar protein sorting 28
SEQ ID NO: 74
GTATTTTGCCCTCCAACTTTGAAGGAAAGGAAAAGGTTTCTAATTGGTTG
AATGTTTTGACAGCTATGTCAGCCAGTGATGAGCTCAATGAAACACAAGT
Gene #59
Vps2 (Partial)
protein transport
SEQ ID NO: 75
GCAAATATTCAAGCAGTATCTTTGAAGATCCAAACTCTTAGATCACAGAA
TGCAATGGCAGAAGCTATGAAAGGTTGTTCTAGAGCTATGGCAAACATGA
ATAGGCAAATGAATTTACCACAAATTCAGCGAATATTATCAGAATTTGAG
AAACAATCAGAAATAATGGACATGAAAGAAGAAATGATGAATGATGCAAT
GGATGATGCCATGGGAGATGATGATGATGAAGAGGAAACGGATGCAGTTG
TTACACAAGTTCTTGATGAATTAGGTCTCCAATTAAATGACCAA
Gene #59
Nucleotides 56-155 of SEQ ID NO: 75
Vps2
protein transport
SEQ ID NO: 76
TGGCAGAAGCTATGAAAGGTTGTTCTAGAGCTATGGCAAACATGAATAGG
CAAATGAATTTACCACAAATTCAGCGAATATTATCAGAATTTGAGAAACA
Gene #60
Vps24 (partial)
Charged multivesicular body protein 3
SEQ ID NO: 77
GGTGATAAAGATGTCTGTGTTACGTTGGCCAAGGAAATTATCAATGCAAG
GAAACATATAACAAAGATTCATACATCAAAAGCCCATCTGAATTCTATAC
AATTGCAAATGAAAAATCAATTATCTTTATTAAGAGTATCTGGATCAATA
CAAAAATCAACAGAAGTTATGCAAGCTATGCAGAATCTAGTAAATGTACC
TGAAGTAGCAAACACAATGAGAGAAATGTCCAAAGAAATGATGAAAGCTG
GAATTATGGAAGAGATGATTGAAGAAACTATGGAATCCTTAGAACCAGAG
GACACTGAAGATATGGAAGAAGAAGCACAAAAAGAAATTGATAAGGTACT
CTGGGACTTAACAGCTGGGGCACTTGGTAAAGTACCGGATGCTGTTAAAG
ATGTACCATCCTCATCT
Gene #60
Nucleotides 1-100 of SEQ ID NO: 77
Vps24
Charged multivesicular body protein 3
SEQ ID NO: 78
GGTGATAAAGATGTCTGTGTTACGTTGGCCAAGGAAATTATCAATGCAAG
GAAACATATAACAAAGATTCATACATCAAAAGCCCATCTGAATTCTATAC
Gene #61
Snf7/shrub (partial)
ESCRT-III pathway
SEQ ID NO: 79
CAGAGAGAAGCTCTGGAAGGTGCCAATACTAATACTGCAGTTCTAACAAC
AATGAAGAATGCTGCAGATGCTCCTAAAGCTGCACACAAACACATGGATG
TTAACCAAGTACACGATATGATGGATGATATTGCTGAACAGCAAGATGTA
GCCAAGGAAATATCTGAAGCCATTTCTAATCCAGTTGCCTTTGGTCATGA
TGTAGATGAGGATGAGTTAGAAAAAGAATTAGAAGAATTAGAACAAGAAG
AATTGGATAAGGATCTGCTTAAACTAAGTACGCCTGGTGATGATCTACCT
GAACTACCATCCACTGCACCAAAAGACAAAGCCAAAAGAAAAAGCTAGGC
ACAAAGGAACGTTCTAGTAGATGATGAAATCAAAGAATTAGAAGCATGGG
CTTCATAA
Gene #61
Nucleotides 261-360 of SEQ ID NO: 79
Snf7/shrub
ESCRT-III pathway
SEQ ID NO: 80
GGATCTGCTTAAACTAAGTACGCCTGGTGATGATCTACCTGAACTACCAT
CCACTGCACCAAAAGACAAAGCCAAAAGAAAAAGCTAGGCACAAAGGAAC

1. An isolated small inhibitory ribonucleic acid molecule (siRNA) that inhibits expression of an essential gene of Gb.

2. The siRNA of claim 1 comprising a unit of a first strand of nucleotides that is substantially identical to at least 17 contiguous nucleotides from said essential gene, and a second strand of nucleotides that is substantially complementary to said first strand of nucleotides.

3. The siRNA of claim 2 wherein said first and second strands of nucleotides are at least about 25, 35, 50, 70, or 100 nucleotides in length.

4. The siRNA of claim 1 wherein over their respective lengths said first and second strands of nucleotides are 70-100% identical to said essential gene.

5.-9. (canceled)

10. A vector comprising an expression control sequence operatively linked to a nucleotide sequence that is a template for one or both strands according to claim 2.

11. A host cell comprising the expression vector of claim 10.

12. A plant tissue comprising the siRNA of claim 1.

13. A plant tissue comprising the vector of claim 10.

14. A plant tissue comprising the host cell of claim 11.

15. A vector comprising an isolated nucleic acid operably linked to an expression control sequence wherein the isolated nucleic acid comprises a sequence that selectively hybridizes under high stringency hybridization conditions to a sequence selected from the group consisting of SEQ ID NO: 30, 1-29, 31-56 and 71-80, and complementary sequences thereof.

16. The vector of claim 15 wherein said nucleic acid is 90-99.99 percent identical to said sequence selected from the group consisting of SEQ ID NO: 1-56 and 71-80, and complementary sequences thereof.

17. The vector of claim 15 wherein said nucleic acid comprises at least 17 contiguous nucleotides of a sequence selected from the group consisting of SEQ ID NO: 1-56 and 71-80, and complementary sequences thereof.

18. The vector of claim 17 wherein said nucleic acid comprises at least 25 contiguous nucleotides of a sequence selected from the group consisting of SEQ ID NO: 1-56 and 71-80, and complementary sequences thereof.

19. The vector of claim 15 wherein said nucleic acid is less than about 80% identical to the honey bee ortholog of said nucleic acid.

20.-21. (canceled)

22. A plant tissue comprising the vector of claim 15.

23.-24. (canceled)

25. An isolated double stranded ribonucleic acid molecule (dsRNA) comprising a unit of a first strand of nucleotides that is substantially identical to at least 17 contiguous nucleotides set forth in SEQ ID NO: 1-56 and 71-80 and a second strand of nucleotides that is substantially complementary to said first strand of nucleotides.

26. (canceled)

27. The dsRNA of claim 25 wherein said first and second strands of nucleotides are at least about 25, 35, 50, 70, or 100 nucleotides in length.

28. The dsRNA of claim 25 wherein over their respective lengths said first and second strands of nucleotides are 70-100% identical to any one of SEQ ID NO: 1-56 and 71-80.

29. The dsRNA of claim 25 wherein the sequences of said first and second strands of nucleotides are less than about 80% identical to the sequence of the honey bee ortholog of said first and second strands of nucleotides.

30.-31. (canceled)

32. The dsRNA of claim 25 further comprising a loop region separating said first strand and said second strand of nucleotides.

33. A vector comprising an expression control sequence operatively linked to a nucleotide sequence that is a template for one or both strands according to claim 25.

34.-35. (canceled)

36. The host cell of claim 33 wherein said host is an Agrobacterium.

37. A plant tissue transformed with the host cell of claim 36.

38. A plant tissue comprising the dsRNA of claim 25.

39. A method of producing a pest resistant plant comprising expressing an siRNA according to claim 1 in said plant or to propagation or reproductive material of said plant.

40. The method of claim 39 wherein said plant is Eucalyptus.

41. The method of claim 39 wherein said pest is Gb.

42. A method of inhibiting a pest infestation comprising cultivating a plant comprising an siRNA according to claim 11, to inhibit said infestation.

43. The method of claim 42 wherein said plant is Eucalyptus.

44. The method of claim 43 wherein said pest is Gb.

45. A method of producing a plant resistant to a plant pathogenic pest comprising:
(a) transforming a plant cell with a recombinant DNA construct or combination of constructs that express the siRNA of claim 1;
(b) regenerating a plant from the transformed plant cell; and
(c) growing the transformed plant cell under conditions suitable for the transcription of said recombinant DNA construct,
said grown transformed plant thus being resistant to said pest compared to an untransformed plant.

46. (canceled)

47. The method of claim 45 wherein said plant is Eucalyptus.

48. The method of claim 47 wherein said pest is Gb.
